(19) United States
(12) Patent Application Publication
(10) Pub. No.: US 2026/0224582 A1
(43) Pub. Date: Aug. 6, 2026

(54) Heterobicyclic amides as inhibitors of CD38

(71) Applicant: Cerevance, Inc., Boston, MA (US)

(72) Inventors: Roland Burli, Boston, MA (US); Kevin Doyle, Boston, MA (US); Andrew Stott, Boston, MA (US)

(73) Assignee: Cerevance, Inc., Boston, MA (US)

(21) Appl. No.: 18/869,006

(22) PCT Filed: May 18, 2023
(86) PCT No.: PCT/GB2023/051308

§ 371 Date: Nov. 25, 2024

(30) Foreign Application Priority Data
May 25, 2022 (GB) 2207721.8

(51) Int. Cl. A61K 31/53; A61K 31/4375; A61K 31/4985; A61K 31/519; A61K 31/5377; A61K 31/541; C07D 471/04; C07D 487/04

(52) U.S. Cl. CPC A61K 31/53; A61K 31/4375; A61K 31/4985; A61K 31/519; A61K 31/5377; A61K 31/541; C07D 471/04; C07D 487/04

(57) ABSTRACT

The present invention provides compounds of formula (I): 5 A 1 X Y N R 2 R 1 A 2 A 3 A 4 n Formula (I) and pharmaceutically acceptable salts, solvates and prodrugs thereof, wherein R¹, R², n, A1, A2, A3, A4, X and Y are as defined in the specification, processes for their preparation, pharmaceutical compositions containing them and their use in therapy, particularly for 10 use in treating disorders associated with CD38 activity.

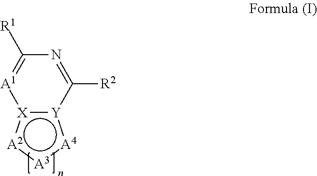

### FIELD OF THE INVENTION

The present invention relates to bicyclic amides and related compounds, processes for their preparation, pharmaceutical compositions containing them and their use in therapy, particularly for use in treating disorders associated with CD38 activity.

### BACKGROUND OF THE INVENTION

### NAD⁺ Homeostasis, Aging & Disease

Nicotinamide adenine dinucleotide (NAD⁺) is an essential cellular component being extremely abundant in most living cells. NAD⁺ and its close analogue NADP⁺ perform similar redox functions within the cell, the latter being more confined to biosynthetic pathways and redox protective roles (Ying, 2008, Antioxid Redox Signal 10:179). NAD⁺ and NADH (NAD (H)) are redox essential for a variety of electron-exchange-dependent biochemical reactions, particularly redox reactions involving oxidoreductase-mediated hydride transfer. Thus NAD (H) plays a vital role in the mitochondrial electron transport chain and cellular energy metabolism and as a co-enzyme linked to catabolism and harvesting of metabolic energy in all eukaryotic cells. However, the roles of NAD⁺ expand beyond its function as a co-enzyme, as NAD⁺ and its metabolites also act as degradation substrates for a wide range of enzymes, such as sirtuins (Hall et al, 2013, J Clin Invest 123:973), SARM1 (Essuman et al, 2017, Neuron 93:1334) and PARP enzymes (Murata et al, 2019, Mol Biol Cell 30:2584). It is through these activities that NAD⁺ also links cellular metabolism to changes in signalling and transcriptional events and thus plays a central role in regulating cellular homeostasis and signalling.

NAD⁺ levels largely remain constant when used as a co-enzyme, but in non-redox reactions its levels are depleted from the cellular pool, thus requiring continuous re-synthesis and replenishment (Nikiforov et al, 2015, Crit Rev Biochem Mol Biol 50:284). There are two main pathways for the synthesis of NAD⁺, the so called de novo pathway that utilizes the essential amino acid L-tryptophan to generate quinolinic acid (QA) that is further metabolized into NAD⁺ (Nikiforov et al, 2015, Crit Rev Biochem Mol Biol 50:284), and the salvage pathway that utilizes nicotinamide (NAM), nicotinic acid (NA), and nicotinamide riboside (NR) (Imai & Yoshino, 2013, Diabetes Obes Metab Suppl. 3: 26). The salvage pathway is the main source of NAD⁺ in most cell types. NAD⁺ levels change during many physiological processes. Mounting evidence indicates that intracellular NAD⁺ levels are significantly affected by nutritional and environmental stimuli. These changes in NAD⁺ content are reflected into NAD⁺-dependent enzymatic activities, which in turn lead to changes in cellular metabolism, gene expression, and protein function. Therefore, maintenance of an optimal NAD⁺ concentration appears critical to maintain long term tissue homeostasis.

It has been clearly demonstrated that cellular NAD⁺ levels decline during chronological aging (Chini et al, 2017, Mol Cell Endocrinol 455:62). This decline appears to play a crucial role in the development of metabolic dysfunction in aging and importantly, decline in cellular NAD⁺ levels has emerged as a potential key player in the pathogenesis of age-related conditions (Chini et al, 2017, Mol Cell Endocrinol 455:62; Verdin, 2015, Science 350:1208; Imai & Guarente, 2014, Trends Cell Biol 24:464; Schultz & Sinclair, 2016, Cell Metab 23:965); thus, maintaining NAD⁺ levels and subsequent cellular homeostasis may be a means of attenuating aging and age-related diseases such as Alzheimer's disease and Parkinson's disease (Chini et al, 2017, Mol Cell Endocrinol 455:62). In support of this many studies have demonstrated that elevated NAD⁺ levels are associated with improved health and longer life span in multiple model organisms and humans (Fang et al, 2016, Cell Metab 24:566; Fang et al, 2019, Nat Comms 10:5284; Lehmann et al, 2017, Biol Open, 6:141; Martens et al, 2018, Nat Comms 9:1286; Mitchell et al, 2018, Cell Metab 27:667; Covarrubias et al, 2021, Nat Rev Mol Cell Biol 22:119; Perez et al, 2021, Mech Ag & Dev 197:111499). As a result, there has been a growing interest in characterizing the role of NAD⁺ metabolism in age-related diseases and in developing pharmacological or nutraceutical interventions that increase NAD⁺ levels. In this regard, restoring NAD⁺ levels with NAD⁺ precursors, such as nicotinamide riboside (NR) and nicotinamide mononucleotide (NMN), has received some clinical interest (for reviews see Covarrubias et al, 2021, Nat Rev Mol Cell Biol 22:119; Perez et al, 2021, Mech Ag & Dev 197:111499) and inhibiting NAD⁺ consumption, e.g. by inhibiting CD38, has emerged as valuable therapeutic approach for age-related disorders and neurological diseases.

### CD38

Cluster of differentiation (CD38) is a multifunctional protein involved in i) cellular and tissue NAD⁺ homeostasis via its hydrolase function (Chini, 2009, Curr Pharm Des 15:57) and ii) the generation of the second messengers ADPR and cyclic-ADPR (cADPR), via CD38s cyclase enzyme activity, that are subsequently involved in intracellular calcium signalling (Lee & Aarhus, 1991, Cell Regul 3:203; Malavasi et al, 2008, Physiol Rev 88:841). CD38 has a type II membrane orientation, with the catalytic site facing the outside of the cell (Chini, 2009, Curr Pharm Des 15:57; Malavasi et al, 2008, Physiol Rev 88:841). This was somewhat of a paradox given most substrates for NADase-CD38 are expected to be intracellular, however, it is now evident that CD38 degrades not only NAD⁺, but also circulating NAD⁺ precursors such as nicotinamide mononucleotide (NMN) and nicotinamide riboside (NR), before they can be incorporated into intracellular NAD⁺ biosynthetic pathways (Yoshino et al, 2018, Cell Metab 27:513). Furthermore, CD38 has also been observed in intracellular membranes, such as in the nuclear membrane, mitochondria, and endoplasmic reticulum (Zhao et al, 2012, Sci Signal 5: ra67; Shrimp et al, 2014, J Am Chem Soc 136:5656), a small fraction of CD38 is also expressed as a type III plasma membrane protein with the catalytic site facing the inside of the cell (Lui et al, 2017, Proc Natl Acad Sci USA. 114:8283), and intra- and extracellular forms of CD38 have also been described (Chini, 2009, Curr Pharm Des 15:57; Malavasi et al, 2008, Physiol Rev 88:841). The relative roles and contributions of the different cellular pools of CD38 in the regulation of NAD⁺ homeostasis, calcium signalling, and subsequent cellular function is thus complex and still not clearly understood. It is apparent that CD38 is a very inefficient second messenger-generating enzyme, as it will hydrolyze almost a hundred molecules of NAD⁺ in order to generate one molecule of cADPR (Beers et al, 1995, J Clin Invest 95:2385; Kim et al, 1993, Science 261:1330), thus its role in NAD⁺ homeostasis may be its primary function reflected by its high substrate affinity and turnover rate compared to other NAD⁺ utilizing enzymes. CD38 is expressed in the brain across species including mouse (Ceni et al, 2003, Biochem J 370:175), rat (Yamada et al, 1997, Brain Res 756:52; Braidy et al, 2014, Biogerontology 15:177) and human (Mizuguchi et al, 1995, Brain Res 697:235). In the human brain, it is interesting to note that CD38 is expressed in virtually all brain areas, with the highest expression levels in the caudate, pallidum, olfactory bulb, putamen, thalamus, and cingulate anterior (Quintana et al, 2019, Nat Comms 10:668). Literature evidence suggests that CD38 is expressed in neurons (Yamada et al, 1997, Brain Res 756:52; Mizuguchi et al, 1995, Brain Res 697:235), astrocytes (Yamada et al, 1997, Brain Res 756:52; Kou et al, 2009, J Neurosci Res 87:2326), and microglial cells (Ma et al, 2012, Biochem Biophys Res Commun 418:714; Mayo et al, 2008, J Immunol 181:92). However, the applicant's in-house data suggest that CD38 expression predominates in the astrocytes of the human forebrain structures.

CD38 function is associated with effects on immunity, metabolic dysfunction, and behavioural deficits in mice (Barbosa et al, 2007, FASEB J 21:3629; Lopatina et al, 2012, Front Neurosci 6:182). Tissue NAD⁺ levels were found to be significantly higher in CD38-deficient mice suggesting that CD38 is the main NAD⁺ metabolising enzyme (NADase) in mammalian tissues. Concurrently it has been demonstrated that the expression and activity of CD38 increases with aging and that CD38 is at least in part the cause for the age-related NAD⁺ decline and subsequent mitochondrial dysfunction (Camacho-Pereira et al, 2016, Cell Metab 23:1127). Furthermore, reduced NAD⁺ levels are a common observation among neurodegenerative diseases including Alzheimer's disease (Sonntag et al, 2017, Sci Rep 7:14038), Parkinson's disease (Wakade et al, 2014, PLOS ONE 9: e109818), amyotrophic lateral sclerosis (Wang et al, 2017, Cell Rep 20:2184), as well as multiple sclerosis (Braidy et al, 2013, Brain Res 1537:267). Thus, attenuating CD38 activity, enhancing cellular levels of NAD⁺ and subsequent modulation of the diverse NAD⁺ related pathways could be a therapeutically viable approach to a range of brain and inflammatory disorders.

### Therapeutic Utility of CD38 Inhibitors

Several experimental data using CD38 knockout mice (KO) mice have demonstrated positive effects of CD38 deletion in models of neurodegeneration (Blacher et al, 2015, Ann Neurol 78:88; Long et al, 2017, Neurochem Res 42:283; Takaso et al, 2020, Sci Rep 10:17795) and neuroinflammation (Choe et al, 2011, PLOS ONE 6: e19046; Raboon et al, 2019, Front Cell Neurosci 13:258; for review see Guerreiro et al, 2020, Cells 9: 471), and a CD38 inhibitor molecule reversed age-related NAD⁺ decline and physiological effects of aging in mice (Tarrago et al, 2018, Cell Metab 27:1081). Crossing of CD38 KO mouse with the APPswePS1DE9 model of Alzheimer's disease mouse model reduced amyloid plaque load and soluble Aβ levels, an effect that correlated with improved functional performance in a Morris water maze behavioural task (Blacher et al, 2015, Ann Neurol 78:88).

In stroke models CD38-deficient mice showed decreased local expression of the proinflammatory cytokines and reduced ischemic injury and neurological deficits (Choe et al, 2011, PLOS ONE 6: e19046), whilst Long et al (Long et al, 2017, Neurochem Res 42:283) showed an amelioration of histological and neurologic outcome following ischemic insult in CD38 KO mice. In models of multiple sclerosis, CD38 deficiency reduced severity of outcome in mouse experimental autoimmune encephalomyelitis (EAE) (Herrmann et al, 2016, Dis Mods Mechs 9:1211) and suppressed neuroinflammation in a mouse model of demyelination (Raboon et al, 2019, Front Cell Neurosci 13:258). Similarly, deletion of CD38 or supplementation of NAD⁺ attenuate axon degeneration in a mouse facial nerve axotomy model (Takaso et al, 2020, Sci Rep 10:17795). Interestingly, a transcriptome-wide association study has identified CD38 as a potential susceptibility gene for Parkinson's disease (Yao et al, 2021, npj Parkinsons Dis 7:79). In addition, CD38 KO mice are protected against obesity and metabolic syndrome (Barbosa et al, 2007, FASEB J 21:3629; Chiang et al, 2015, PLOS ONE 10: e0134927) which are recognised risk factors for Alzheimer's disease. The regulatory impact of CD38 on the immune cells of the brain and periphery are also likely to be contributors to the beneficial impact of CD38 deletion or blockade on the various preclinical insult models (for reviews see Guerreiro et al, 2020, Cells 9:471; Piedra-Quintero et al, 2020, Front Immunol 11:597959) as neuroinflammation has been shown to be a major contributor across many of these diseases (Ransohoff, 2016, Science 353:777).

Taken together, there is significant preclinical evidence to support the utility of augmenting cellular NAD⁺ levels by inhibiting its breakdown via blockade of CD38. The therapeutic utility in CNS diseases such as Alzheimer's disease, Parkinson's disease, multiple sclerosis, stroke and other neurodegenerative conditions will be reliant on achieving CNS penetration by the CD38 inhibitors. However, CD38 inhibitors will also likely have utility in other conditions such as autoimmune diseases, obesity and metabolic syndrome.

### Brain Permeability of Small Molecules

The central nervous system (CNS) is shielded from exposure to undesired substances by the blood-brain barrier (BBB). This restriction protects neurons from harmful interactions with toxins and other potentially harmful molecules. The BBB consists of brain capillary endothelial cells which have several unique attributes and functions: they have tight junctions leading to extremely low permeability via a paracellular route, they have low rates of endocytosis and, importantly, they highly express efflux transporter proteins with the specific function of recognizing and shuttling foreign substances out of the CNS (Gloor et al, 2001, Brain Res Rev 36:258).

Importantly, the unbound drug concentration in the brain compartment is a critical parameter that needs to be considered when evaluating the suitability of molecules as potential therapies for neurological and neurodegenerative disorders: it is generally accepted that only the unbound fraction of drug may be available for occupying the desired target in order to exert a pharmacological effect.

There is a need for treatment of the above diseases and conditions and others described herein with compounds that are CD38 inhibitors. The present invention provides such CD38 inhibitors, including brain permeable CD38 inhibitors.

### SUMMARY OF THE INVENTION

A first aspect of the present invention provides a compound of formula (I):

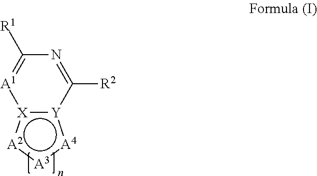

or a pharmaceutically acceptable salt, solvate or prodrug thereof, wherein:
one of R¹ and R² is —C(O)NHR³ and the other one of R¹ and R² is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
R³ is a saturated 3- to 9-membered carbocyclic or heterocyclic group optionally substituted with one or more substituents independently selected from —NR⁴R⁵ and C₁-C₃ alkyl, wherein the C₁-C₃ alkyl is optionally substituted with one or more substituents independently selected from halo, hydroxyl and C₁-C₃ alkoxy;
R⁴ is hydrogen or C₁-C₃ alkyl;
R⁵ is C₁-C₄ alkyl (preferably C₁-C₃ alkyl) optionally substituted with one or more substituents independently selected from halo, hydroxyl and C₁-C₃ alkoxy;
or R⁴ and R⁵ together with the nitrogen atom to which they are attached form a saturated 3- to 6-membered heterocyclic group, wherein the saturated 3- to 6-membered heterocyclic group is optionally substituted with one or more substituents independently selected from halo, hydroxyl, C₁-C₃ alkyl, C₁-C₃ haloalkyl, C₁-C₃ alkoxy and oxo (═O) [preferably independently selected from halo, hydroxyl, C₁-C₃ alkoxy and oxo (═O)];
each of A¹, A², A³ and A⁴ is independently selected from N and CH;
each X and Y is independently selected from N and C; and
n is 1 or 2;
   provided that:
when n is 1, one of X and Y is N and the other one of X and Y is C; and
when n is 2, X and Y are C and at least one of A², A³ and A⁴ is N.

In one embodiment of the first aspect of the present invention, the compound is of Formula (Ia):

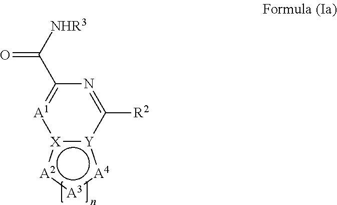

or a pharmaceutically acceptable salt, solvate or prodrug thereof, wherein:

R² is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
R³ is a saturated 3- to 9-membered carbocyclic or heterocyclic group optionally substituted with one or more substituents independently selected from —NR⁴R⁵ and C₁-C₃ alkyl, wherein the C₁-C₃ alkyl is optionally substituted with one or more substituents independently selected from halo, hydroxyl and C₁-C₃ alkoxy;
R⁴ is hydrogen or C₁-C₃ alkyl;
R⁵ is C₁-C₄ alkyl (preferably C₁-C₃ alkyl) optionally substituted with one or more substituents independently selected from halo, hydroxyl and C₁-C₃ alkoxy;
or R⁴ and R⁵ together with the nitrogen atom to which they are attached form a saturated 3- to 6-membered heterocyclic group, wherein the saturated 3- to 6-membered heterocyclic group is optionally substituted with one or more substituents independently selected from halo, hydroxyl, C₁-C₃ alkyl, C₁-C₃ haloalkyl, C₁-C₃ alkoxy and oxo (═O) [preferably independently selected from halo, hydroxyl, C₁-C₃ alkoxy and oxo (═O)];
each of A¹, A², A³ and A⁴ is independently selected from N and CH;
each X and Y is independently selected from N and C; and
n is 1 or 2;
   provided that:
when n is 1, one of X and Y is N and the other one of X and Y is C; and
when n is 2, X and Y are C and at least one of A², A³ and A⁴ is N.

In another embodiment of the first aspect of the present invention, the compound is of Formula (Ib):

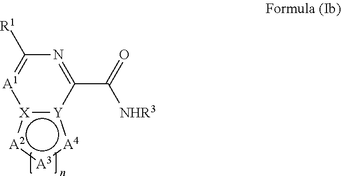

or a pharmaceutically acceptable salt, solvate or prodrug thereof, wherein:
R¹ is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
R³ is a saturated 3- to 9-membered carbocyclic or heterocyclic group optionally substituted with one or more substituents independently selected from —NR⁴R⁵ and C₁-C₃ alkyl, wherein the C₁-C₃ alkyl is optionally substituted with one or more substituents independently selected from halo, hydroxyl and C₁-C₃ alkoxy;
R⁴ is hydrogen or C₁-C₃ alkyl;
R⁵ is C₁-C₄ alkyl (preferably C₁-C₃ alkyl) optionally substituted with one or more substituents independently selected from halo, hydroxyl and C₁-C₃ alkoxy;
or R⁴ and R⁵ together with the nitrogen atom to which they are attached form a saturated 3- to 6-membered heterocyclic group, wherein the saturated 3- to 6-membered heterocyclic group is optionally substituted with one or more substituents independently selected from halo, hydroxyl, C₁-C₃ alkyl, C₁-C₃ haloalkyl, C₁-C₃ alkoxy and oxo (═O) [preferably independently selected from halo, hydroxyl, C₁-C₃ alkoxy and oxo (═O)];
each of A¹, A², A³ and A⁴ is independently selected from N and CH;
each X and Y is independently selected from N and C; and
n is 1 or 2;
   provided that:
when n is 1, one of X and Y is N and the other one of X and Y is C; and
when n is 2, X and Y are C and at least one of A², A³ and A⁴ is N.

In the compounds of formula (I), (Ia) and (Ib), nis 1 or 2.

In one embodiment, n is 1, such that the compounds are of formula (I′), (Ia′) or (Ib′):

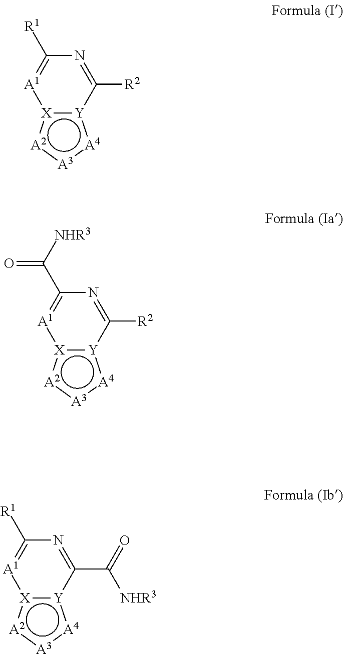

wherein one of X and Y is N and the other one of X and Y is C.

In the compounds of formula (I′), (Ia′) or (Ib′), and salts, solvates and prodrugs thereof, each of A¹, A², A³ and A⁴ is independently selected from N and CH. In one embodiment, each of A¹, A², A³ and A⁴ is CH. In one embodiment, one, two or three of A¹, A², A³ and A⁴ are N and the remaining of A¹, A², A³ and A⁴ are CH. In a preferred embodiment, one or two of A¹, A², A³ and A⁴ are N and the remaining of A¹, A², A³ and A⁴ are CH. In a preferred embodiment, A³ is CH.

In another embodiment, n is 2, such that the compounds are of formula (I″), (Ia″) or (Ib″):

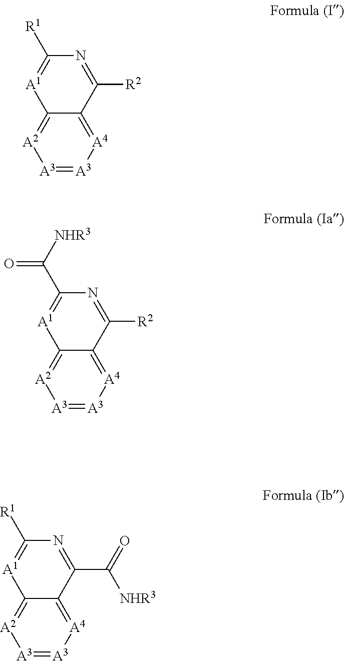

wherein at least one of A², A³ and A⁴ is N.

In the compounds of formula (I″), (Ia″) or (Ib″), and salts, solvates and prodrugs thereof, each of A¹, A², A³ and A⁴ is independently selected from N and CH, wherein at least one of A², A³ and A⁴ is N. In one embodiment, one, two or three of A¹, A², A³ and A⁴ are N and the remaining of A¹, A², A³ and A⁴ are CH, and at least one of A², A³ and A⁴ is N. In a preferred embodiment, one or two of A¹, A², A³ and A⁴ are N and the remaining of A¹, A², A³ and A⁴ are CH, and at least one of A², A³ and A⁴ is N. In a preferred embodiment, A³ is CH.

In the compounds of the first aspect of the present invention (that is, the compounds of formula (I), (Ia), (Ib), (I′), (Ia′), (Ib′), (I″), (Ia″) or (Ib″), or a pharmaceutically acceptable salt, solvate or prodrug thereof), the 5-membered heteroaryl group of R¹ or R² containing one, two or three heteroatoms independently selected from N and S, may be selected from pyrrolyl, thiophenyl, pyrazolyl, imidazolyl, thiazolyl, isothiazolyl, triazolyl (including 1,2,3-triazolyl and 1,2,4-triazolyl), and thiadiazolyl (including 1,2,3-thiadiazolyl, 1,2,4-thiadiazolyl, 1,2,5-thiadiazolyl, and 1,3,4-thiadiazolyl).

In one embodiment, the 5-membered heteroaryl group of R¹ or R² is selected from pyrazolyl, imidazolyl, thiazolyl, isothiazolyl, triazolyl (including 1,2,3-triazolyl and 1,2,4-triazolyl), and thiadiazolyl (including 1,2,3-thiadiazolyl, 1,2,4-thiadiazolyl, 1,2,5-thiadiazolyl, and 1,3,4-thiadiazolyl).

In another embodiment, the 5-membered heteroaryl group of R¹ or R² is selected from pyrazolyl, imidazolyl, thiazolyl, isothiazolyl, and triazolyl (including 1,2,3-triazolyl and 1,2,4-triazolyl).

In a preferred embodiment, the 5-membered heteroaryl group of R¹ or R² is selected from pyrazolyl, imidazolyl, thiazolyl, and isothiazolyl.

In a further preferred embodiment, the 5-membered heteroaryl group of R¹ or R² is imidazolyl.

In the compounds of the first aspect of the present invention, the 5-membered heteroaryl group of R¹ or R² is optionally substituted with one or more (such as one, two or three) substituents independently selected from C₁-C₃ alkyl, wherein the C₁-C₃ alkyl is optionally substituted with one or more (such as one, two, three, four or five) substituents independently selected from halo, hydroxyl and C₁-C₃ alkoxy.

In one embodiment, the 5-membered heteroaryl group of R¹ or R² is optionally substituted with one or two substituents independently selected from C₁-C₃ alkyl, wherein the C₁-C₃ alkyl is optionally substituted with one, two, three, four or five halo substituents or with one substituent selected from hydroxyl and C₁-C₃ alkoxy.

In another embodiment, the 5-membered heteroaryl group of R¹ or R² is optionally substituted with one substituent selected from C₁-C₂ alkyl, wherein the C₁-C₂ alkyl is optionally substituted with one, two or three halo substituents or with one substituent selected from hydroxyl, methoxy and ethoxy.

In a preferred embodiment, the 5-membered heteroaryl group of R¹ or R² is optionally substituted with one substituent selected from C₁-C₂ alkyl, wherein the C₁-C₂ alkyl is optionally substituted with one hydroxyl substituent.

In another preferred embodiment, the 5-membered heteroaryl group of R¹ or R² is optionally substituted with one substituent selected from —CH₃ or —CH₂CH₂OH.

In yet another preferred embodiment, the 5-membered heteroaryl group of R¹ or R² is unsubstituted.

In the compounds of the first aspect of the present invention, the saturated 3- to 9-membered carbocyclic or heterocyclic group of R³ may be selected from monocyclic carbocyclic groups, bicyclic (including bridged, fused and spiro) carbocyclic groups, monocyclic heterocyclic groups, and bicyclic (including bridged, fused and spiro) heterocyclic groups.

In one embodiment, the saturated 3- to 9-membered carbocyclic or heterocyclic group of R³ is selected from cyclopropyl, cyclobutyl, cyclopentyl, cyclohexyl, cycloheptyl, cyclooctyl, azetidinyl, oxetanyl, thietanyl, pyrrolidinyl, tetrahydrofuranyl, tetrahydrothiophenyl, pyrazolidinyl, imidazolidinyl, oxazolidinyl, isoxazolidinyl, dioxolanyl, oxathiolanyl, piperidinyl, tetrahydropyranyl, thianyl, piperazinyl, dioxanyl, morpholinyl, thiomorpholinyl, spiro[2.2]pentanyl, spiro[2.3]hexanyl, spiro[2.4]heptanyl, spiro[3.3]heptanyl, spiro[2.5]octanyl, spiro[3.4]octanyl, spiro[2.6]nonanyl, spiro[3.5]nonanyl, spiro[4.4]nonanyl, azaspiro[2.2]pentanyl, azaspiro[2.3]hexanyl, azaspiro[2.4]heptanyl, azaspiro[3.3]heptanyl, azaspiro[2.5]octanyl, azaspiro[3.4]octanyl, azaspiro[2.6]nonanyl, azaspiro[3.5]nonanyl, azaspiro[4.4]nonanyl, oxaspiro[2.2]pentanyl, oxaspiro[2.3]hexanyl, oxaspiro[3.3]heptanyl, oxaspiro[2.5]octanyl, oxaspiro[2.4]heptanyl, oxaspiro[3.4]octanyl, oxaspiro[2.6]nonanyl, oxaspiro[3.5]nonanyl, oxaspiro[4.4]nonanyl, quinuclidinyl, 8-azabicyclo[3.2.1]octanyl, 2-azabicyclo[2.2.2]octanyl, and hexahydro-1H-pyrrolizinyl.

In another embodiment, the saturated 3- to 9-membered carbocyclic or heterocyclic group of R³ is selected from cyclopropyl, cyclobutyl, cyclopentyl, cyclohexyl, cycloheptyl, cyclooctyl, azetidinyl, oxetanyl, thietanyl, pyrrolidinyl, tetrahydrofuranyl, tetrahydrothiophenyl, pyrazolidinyl, imidazolidinyl, oxazolidinyl, isoxazolidinyl, dioxolanyl, oxathiolanyl, piperidinyl, tetrahydropyranyl, thianyl, piperazinyl, dioxanyl, morpholinyl, thiomorpholinyl, spiro[2.3]hexanyl, spiro[2.4]heptanyl, spiro[3.3]heptanyl, spiro[2.5]octanyl, spiro[3.4]octanyl, spiro[2.6]nonanyl, spiro[3.5]nonanyl, spiro[4.4]nonanyl, azaspiro[2.3]hexanyl, azaspiro[2.4]heptanyl, azaspiro[3.3]heptanyl, azaspiro[2.5]octanyl, azaspiro[3.4]octanyl, azaspiro[2.6]nonanyl, azaspiro[3.5]nonanyl, and azaspiro[4.4]nonanyl.

In a preferred embodiment, the saturated 3- to 9-membered carbocyclic or heterocyclic group of R³ is selected from cyclopropyl, cyclobutyl, cyclopentyl, cyclohexyl, cycloheptyl, azetidinyl, oxetanyl, pyrrolidinyl, tetrahydrofuranyl, pyrazolidinyl, imidazolidinyl, oxazolidinyl, isoxazolidinyl, dioxolanyl, piperidinyl, tetrahydropyranyl, piperazinyl, dioxanyl, morpholinyl, spiro[2.4]heptanyl, spiro[3.3]heptanyl, spiro[2.5]octanyl, spiro[3.4]octanyl, spiro[2.6]nonanyl, spiro[3.5]nonanyl, spiro[4.4]nonanyl, azaspiro[2.4]heptanyl, azaspiro[3.3]heptanyl, azaspiro[2.5]octanyl, azaspiro[3.4]octanyl, azaspiro[2.6]nonanyl, and azaspiro[3.5]nonanyl, azaspiro[4.4]nonanyl.

In another preferred embodiment, the saturated 3- to 9-membered carbocyclic or heterocyclic group of R³ is selected from cyclobutyl, cyclopentyl, cyclohexyl, pyrrolidinyl, piperidinyl, tetrahydropyranyl, spiro[3.3]heptanyl, and azaspiro[3.5]nonanyl.

In yet another preferred embodiment, the saturated 3- to 9-membered carbocyclic or heterocyclic group of R³ is cyclohexyl.

When R³ is cyclohexyl, the cyclohexyl preferably has a single substituent which is a trans substituent in the 4-position.

In the compounds of the first aspect of the present invention, the saturated 3- to 9-membered carbocyclic or heterocyclic group of R³ is optionally substituted with one or more (such as one, two or three) substituents independently selected from —NR⁴R⁵ and C₁-C₃ alkyl, wherein the C₁-C₃ alkyl is optionally substituted with one or more (such as one, two, three, four or five) substituents independently selected from halo, hydroxyl and C₁-C₃ alkoxy; wherein:
R⁴ is hydrogen or C₁-C₃ alkyl;
R⁵ is C₁-C₄ alkyl (preferably C₁-C₃ alkyl) optionally substituted with one or more (such as one, two, three, four or five) substituents independently selected from halo, hydroxyl and C₁-C₃ alkoxy;
or R⁴ and R⁵ together with the nitrogen atom to which they are attached form a saturated 3- to 6-membered heterocyclic group, wherein the saturated 3- to 6-membered heterocyclic group is optionally substituted with one or more (such as one, two, three, four or five) substituents independently selected from halo, hydroxyl, C₁-C₃ alkyl, C₁-C₃ haloalkyl, C₁-C₃ alkoxy and oxo (═O) [preferably independently selected from halo, hydroxyl, C₁-C₃ alkoxy and oxo (═O)].

In one embodiment, the saturated 3- to 9-membered carbocyclic or heterocyclic group of R³ is optionally substituted with one or two substituents. In a preferred embodiment, the saturated 3- to 9-membered carbocyclic or heterocyclic group of R³ is optionally substituted with one substituent.

The substituent(s) on the saturated 3- to 9-membered carbocyclic or heterocyclic group of R³ may be selected from C₁-C₃ alkyl, wherein the C₁-C₃ alkyl is optionally substituted with one, two, three, four or five substituents independently selected from halo, hydroxyl and C₁-C₃ alkoxy.

In one embodiment, the substituent(s) on the saturated 3- to 9-membered carbocyclic or heterocyclic group of R³ is selected from C₁-C₃ alkyl, wherein the C₁-C₃ alkyl is optionally substituted with one, two, three, four or five halo substituents or with one substituent selected from hydroxyl and C₁-C₃ alkoxy.

In a preferred embodiment, the substituent(s) on the saturated 3- to 9-membered carbocyclic or heterocyclic group of R³ is selected from C₁-C₂ alkyl, wherein the C₁-C₂ alkyl is optionally substituted with one, two or three halo substituents or with one substituent selected from hydroxyl, methoxy and ethoxy.

In another preferred embodiment, the substituent(s) on the saturated 3- to 9-membered carbocyclic or heterocyclic group of R³ is —CH₂CF₃.

The substituent(s) on the saturated 3- to 9-membered carbocyclic or heterocyclic group of R³ may be selected from —NR⁴R⁵, wherein:
R⁴ is hydrogen or C₁-C₃ alkyl; and
R⁵ is C₁-C₄ alkyl (preferably C₁-C₃ alkyl) optionally substituted with one, two, three, four or five substituents independently selected from halo, hydroxyl and C₁-C₃ alkoxy.

Preferably any hydroxyl and C₁-C₃ alkoxy substituents on R⁵ are not directly attached to the same carbon atom as the nitrogen atom of —NR⁴R⁵.

In one embodiment, the substituent(s) on the saturated 3- to 9-membered carbocyclic or heterocyclic group of R³ is —NR⁴R⁵, wherein:
R⁴ is hydrogen or C₁-C₃ alkyl; and
R⁵ is C₁-C₄ alkyl (preferably C₁-C₃ alkyl) optionally substituted with one, two, three, four or five halo substituents or with one substituent selected from hydroxyl and C₁-C₃ alkoxy.

In a preferred embodiment, the substituent(s) on the saturated 3- to 9-membered carbocyclic or heterocyclic group of R³ is —NR⁴R⁵, wherein:
R⁴ is hydrogen or methyl; and
R⁵ is C₁-C₄ alkyl (preferably C₁-C₃ alkyl, more preferably C₁-C₂ alkyl) optionally substituted with one, two or three halo substituents or with one substituent selected from hydroxyl, methoxy and ethoxy.

In another preferred embodiment, the substituent(s) on the saturated 3- to 9-membered carbocyclic or heterocyclic group of R³ is —NH-fluoroethyl [including —NH(CH₂CF₃) and —NH(CH₂CHF₂)], —NH-fluoropropyl [including —NH(CH₂CF₂CH₃)], —NH-fluorobutyl [including —NH(CMe₂CF₃)], —NMe-fluoroethyl [including —NMe(CH₂CF₃) and —NMe(CH₂CHF₂)], —NMe-fluoropropyl [including —NMe(CH₂CF₂CH₃)], or —NMe-fluorobutyl [including —NMe(CMe₂CF₃)].

In yet another preferred embodiment, the substituent(s) on the saturated 3- to 9-membered carbocyclic or heterocyclic group of R³ is —NH(CH₂CF₃) or —NH(CH₂CHF₂).

The substituent(s) on the saturated 3- to 9-membered carbocyclic or heterocyclic group of R³ may be selected from —NR⁴R⁵, wherein R⁴ and R⁵ together with the nitrogen atom to which they are attached form a saturated 3- to 6-membered heterocyclic group, wherein the saturated 3- to 6-membered heterocyclic group is optionally substituted with one, two, three, four or five substituents independently selected from halo, hydroxyl, C₁-C₃ alkyl, C₁-C₃ haloalkyl, C₁-C₃ alkoxy and oxo (═O) [preferably independently selected from halo, hydroxyl, C₁-C₃ alkoxy and oxo (═O)].

In one embodiment, the substituent(s) on the saturated 3- to 9-membered carbocyclic or heterocyclic group of R³ is selected from azetidinyl, pyrrolidinyl, pyrazolidinyl, imidazolidinyl, oxazolidinyl, isoxazolidinyl, thiazolidinyl, isothiazolidinyl, piperidinyl, piperazinyl, hexahydropyrimidinyl, hexahydropyridazinyl, morpholinyl, 1,3-oxazinanyl, 1,2-oxazinanyl, thiomorpholinyl, 1,3-thiazinanyl, and 1,2-thiazinanyl, each of which is optionally substituted with one, two, three, four or five substituents independently selected from halo, hydroxyl, C₁-C₃ alkyl, C₁-C₃ haloalkyl, C₁-C₃ alkoxy and oxo (═O) [preferably independently selected from halo, hydroxyl, C₁-C₃ alkoxy and oxo (═O)].

In one embodiment, the substituent(s) on the saturated 3- to 9-membered carbocyclic or heterocyclic group of R³ is selected from azetidinyl, pyrrolidinyl, pyrazolidinyl, imidazolidinyl, oxazolidinyl, isoxazolidinyl, thiazolidinyl, isothiazolidinyl, piperidinyl, piperazinyl, hexahydropyrimidinyl, hexahydropyridazinyl, morpholinyl, 1,3-oxazinanyl, 1,2-oxazinanyl, thiomorpholinyl, 1,3-thiazinanyl, and 1,2-thiazinanyl, each of which is optionally substituted with one, two, three or four substituents independently selected from halo, hydroxyl, C₁-C₃ alkyl, C₁-C₃ haloalkyl, C₁-C₃ alkoxy and oxo (═O) [preferably independently selected from halo, hydroxyl, C₁-C₃ alkoxy and oxo (═O)].

In another embodiment, the substituent(s) on the saturated 3- to 9-membered carbocyclic or heterocyclic group of R³ is selected from azetidinyl, pyrrolidinyl, pyrazolidinyl, imidazolidinyl, oxazolidinyl, isoxazolidinyl, piperidinyl, piperazinyl, morpholinyl, and thiomorpholinyl, each of which is optionally substituted with one, two, three, four or five substituents independently selected from halo, hydroxyl, C₁-C₃ alkyl, C₁-C₃ haloalkyl, C₁-C₃ alkoxy and oxo (═O) [preferably independently selected from halo, hydroxyl, C₁-C₃ alkoxy and oxo (═O)].

In another embodiment, the substituent(s) on the saturated 3- to 9-membered carbocyclic or heterocyclic group of R³ is selected from azetidinyl, pyrrolidinyl, pyrazolidinyl, imidazolidinyl, oxazolidinyl, isoxazolidinyl, piperidinyl, piperazinyl, morpholinyl, and thiomorpholinyl, each of which is optionally substituted with one, two, three, four or five halo substituents, with one or two oxo substituents or with one substituent selected from hydroxyl and C₁-C₃ alkoxy.

In a preferred embodiment, the substituent(s) on the saturated 3- to 9-membered carbocyclic or heterocyclic group of R³ is selected from azetidinyl, pyrrolidinyl, imidazolidinyl, oxazolidinyl, piperidinyl, piperazinyl, morpholinyl, and thiomorpholinyl, each of which is optionally substituted with one, two or three halo substituents, with one or two oxo substituents or with one substituent selected from hydroxyl, methoxy and ethoxy.

In another preferred embodiment, the substituent(s) on the saturated 3- to 9-membered carbocyclic or heterocyclic group of R³ is selected from pyrrolidin-1-yl, morpholin-4-yl and thiomorpholin-4-yl, each of which is optionally substituted with one or two halo or oxo substituents or with one hydroxyl substituent.

In a first specific embodiment of the first aspect, the present invention provides a compound of formula (I), or a pharmaceutically acceptable salt, solvate or prodrug thereof, wherein:
one of R¹ and R² is —C(O)NHR³ and the other one of R¹ and R² is selected from pyrazolyl, imidazolyl, thiazolyl, isothiazolyl, triazolyl (including 1,2,3-triazolyl and 1,2,4-triazolyl), and thiadiazolyl (including 1,2,3-thiadiazolyl, 1,2,4-thiadiazolyl, 1,2,5-thiadiazolyl, and 1,3,4-thiadiazolyl), each of which is optionally substituted with one or two substituents independently selected from C₁-C₃ alkyl, wherein the C₁-C₃ alkyl is optionally substituted with one, two, three, four or five halo substituents or with one substituent selected from hydroxyl and C₁-C₃ alkoxy;
R³ is selected from cyclopropyl, cyclobutyl, cyclopentyl, cyclohexyl, cycloheptyl, cyclooctyl, azetidinyl, thietanyl, oxetanyl, pyrrolidinyl, tetrahydrofuranyl, tetrahydrothiophenyl, pyrazolidinyl, imidazolidinyl, oxazolidinyl, isoxazolidinyl, dioxolanyl, oxathiolanyl, piperidinyl, tetrahydropyranyl, thianyl, piperazinyl, dioxanyl, spiro[2.3]hexanyl, spiro[2.4]heptanyl, morpholinyl, thiomorpholinyl, spiro[3.3]heptanyl, spiro[2.5]octanyl, spiro[3.4]octanyl, spiro[2.6]nonanyl, spiro[3.5]nonanyl, spiro[4.4]nonanyl, azaspiro[2.3]hexanyl, azaspiro[2.4]heptanyl, azaspiro[3.3]heptanyl, azaspiro[2.5]octanyl, azaspiro[3.4]octanyl, azaspiro[2.6]nonanyl, azaspiro[3.5]nonanyl, and azaspiro[4.4]nonanyl, each of which is optionally substituted with one or two substituents independently selected from:
   (i) C₁-C₃ alkyl optionally substituted with one, two, three, four or five halo substituents or with one substituent selected from hydroxyl and C₁-C₃ alkoxy;
   (ii) —NR⁴R⁵, wherein:
      R⁴ is hydrogen or C₁-C₃ alkyl; and
      R⁵ is C₁-C₄ alkyl (preferably C₁-C₃ alkyl) optionally substituted with one, two, three, four or five halo substituents or with one substituent selected from hydroxyl and C₁-C₃ alkoxy; and
   (iii) azetidinyl, pyrrolidinyl, pyrazolidinyl, imidazolidinyl, oxazolidinyl, isoxazolidinyl, piperidinyl, piperazinyl, morpholinyl, and thiomorpholinyl, each of which is optionally substituted with one, two, three, four or five substituents independently selected from halo, hydroxyl, C₁-C₃ alkyl, C₁-C₃ haloalkyl, C₁-C₃ alkoxy and oxo (═O) [preferably independently selected from halo, hydroxyl, C₁-C₃ alkoxy and oxo (═O)];
   each of A¹, A², A³ and A⁴ is independently selected from N and CH;
   each X and Y is independently selected from N and C; and
   n is 1 or 2;
      provided that:
when n is 1, one of X and Y is N and the other one of X and Y is C; and
when n is 2, X and Y are C and at least one of A², A³ and A⁴ is N.

In a second specific embodiment of the first aspect, the present invention provides a compound of formula (I), or a pharmaceutically acceptable salt, solvate or prodrug thereof, wherein:
one of R¹ and R² is —C(O)NHR³ and the other one of R¹ and R² is selected from pyrazolyl, imidazolyl, thiazolyl, isothiazolyl, triazolyl (including 1,2,3-triazolyl and 1,2,4-triazolyl), and thiadiazolyl (including 1,2,3-thiadiazolyl, 1,2,4-thiadiazolyl, 1,2,5-thiadiazolyl, and 1,3,4-thiadiazolyl), each of which is optionally substituted with one or two substituents independently selected from C₁-C₃ alkyl, wherein the C₁-C₃ alkyl is optionally substituted with one, two, three, four or five halo substituents or with one substituent selected from hydroxyl and C₁-C₃ alkoxy;
R³ is selected from cyclopropyl, cyclobutyl, cyclopentyl, cyclohexyl, cycloheptyl, cyclooctyl, azetidinyl, oxetanyl, thietanyl, pyrrolidinyl, tetrahydrofuranyl, tetrahydrothiophenyl, pyrazolidinyl, imidazolidinyl, oxazolidinyl, isoxazolidinyl, dioxolanyl, oxathiolanyl, piperidinyl, tetrahydropyranyl, thianyl, piperazinyl, dioxanyl, morpholinyl, thiomorpholinyl, spiro[2.3]hexanyl, spiro[2.4]heptanyl, spiro[3.3]heptanyl, spiro[2.5]octanyl, spiro[3.4]octanyl, spiro[2.6]nonanyl, spiro[3.5]nonanyl, spiro[4.4]nonanyl, azaspiro[2.3]hexanyl, azaspiro[2.4]heptanyl, azaspiro[3.3]heptanyl, azaspiro[2.5]octanyl, azaspiro[3.4]octanyl, azaspiro[2.6]nonanyl, azaspiro[3.5]nonanyl, and azaspiro[4.4]nonanyl, each of which is optionally substituted with one or two substituents independently selected from:
   (i) C₁-C₃ alkyl optionally substituted with one, two, three, four or five halo substituents or with one substituent selected from hydroxyl and C₁-C₃ alkoxy;
   (ii) —NR⁴R⁵, wherein:
      R⁴ is hydrogen or C₁-C₃ alkyl; and
      R⁵ is C₁-C₄ alkyl (preferably C₁-C₃ alkyl) optionally substituted with one, two, three, four or five halo substituents or with one substituent selected from hydroxyl and C₁-C₃ alkoxy; and
   (iii) azetidinyl, pyrrolidinyl, pyrazolidinyl, imidazolidinyl, oxazolidinyl, isoxazolidinyl, piperidinyl, piperazinyl, morpholinyl, and thiomorpholinyl, each of which is optionally substituted with one, two, three, four or five halo substituents, with one or two oxo substituents or with one substituent selected from hydroxyl and C₁-C₃ alkoxy;
each of A¹, A², A³ and A⁴ is independently selected from N and CH;
each X and Y is independently selected from N and C; and
n is 1 or 2;
   provided that:
when n is 1, one of X and Y is N and the other one of X and Y is C; and
when n is 2, X and Y are C and at least one of A², A³ and A⁴ is N.

In a third specific embodiment of the first aspect, the present invention provides a compound of formula (I), or a pharmaceutically acceptable salt, solvate or prodrug thereof, wherein:
one of R¹ and R² is —C(O)NHR³ and the other one of R¹ and R² is selected from pyrazolyl, imidazolyl, thiazolyl, isothiazolyl, and triazolyl (including 1,2,3-triazolyl and 1,2,4-triazolyl), each of which is optionally substituted with one substituent selected from C₁-C₂ alkyl, wherein the C₁-C₂ alkyl is optionally substituted with one, two or three halo substituents or with one substituent selected from hydroxyl, methoxy and ethoxy;
R³ is selected from cyclobutyl, cyclopentyl, cyclohexyl, pyrrolidinyl, piperidinyl, tetrahydropyranyl, spiro[3.3]heptanyl, and azaspiro[3.5]nonanyl, each of which is optionally substituted with one substituent selected from:
   (i) C₁-C₂ alkyl optionally substituted with one, two or three halo substituents or with one substituent selected from hydroxyl, methoxy and ethoxy;
   (ii) —NR⁴R⁵, wherein:
      R⁴ is hydrogen or methyl; and
      R⁵ is C₁-C₂ alkyl optionally substituted with one, two or three halo substituents or with one substituent selected from hydroxyl, methoxy and ethoxy; and
   (iii) azetidinyl, pyrrolidinyl, imidazolidinyl, oxazolidinyl, piperidinyl, piperazinyl, morpholinyl, and thiomorpholinyl, each of which is optionally substituted with one, two or three halo substituents, with one or two oxo substituents or with one substituent selected from hydroxyl, methoxy and ethoxy;
one or two of A¹, A², A³ and A⁴ are N and the remaining of A¹, A², A³ and A⁴ are CH;
each X and Y is independently selected from N and C; and
n is 1 or 2;
   provided that:
when n is 1, one of X and Y is N and the other one of X and Y is C; and
when n is 2, X and Y are C and at least one of A², A³ and A⁴ is N.

In a fourth specific embodiment of the first aspect, the present invention provides a compound of formula (I), or a pharmaceutically acceptable salt, solvate or prodrug thereof, wherein:
one of R¹ and R² is —C(O)NHR³ and the other one of R¹ and R² is imidazolyl;
R³ is cyclohexyl optionally substituted with one substituent selected from —CH₂CF₃, —NH(CH₂CF₃), —NH(CH₂CHF₂), pyrrolidin-1-yl, morpholin-4-yl and thiomorpholin-4-yl, wherein the pyrrolidin-1-yl, morpholin-4-yl and thiomorpholin-4-yl is optionally substituted with one or two halo or oxo substituents or with one hydroxyl substituent;
one or two of A¹, A² and A⁴ are N and the remaining of A¹, A² and A⁴ are CH;
A³ is CH;
each X and Y is independently selected from N and C; and
n is 1 or 2;
provided that:
when n is 1, one of X and Y is N and the other one of X and Y is C; and
when n is 2, X and Y are C and at least one of A² and A⁴ is N.

A second aspect of the present invention provides a compound of formula (I) selected from:
7-(1H-imidazol-1-yl)-N-(tetrahydro-2H-pyran-4-yl) imidazo[1,2-c]pyrimidine-5-carboxamide;
7-(1H-imidazol-1-yl)-N-((1r,4r)-4-((2,2,2-trifluoroethyl)amino)cyclohexyl) imidazo[1,2-c]pyrimidine-5-carboxamide;
2-(1H-imidazol-1-yl)-N-(tetrahydro-2H-pyran-4-yl)pyrido[2,3-d]pyrimidine-4-carboxamide;
2-(1H-imidazol-1-yl)-N-((1r,4r)-4-((2,2,2-trifluoroethyl)amino)cyclohexyl)pyrido[2,3-d]pyrimidine-4-carboxamide;
3-(thiazol-5-yl)-N-((1r,4r)-4-((2,2,2-trifluoroethyl)amino)cyclohexyl)pyrrolo[1,2-a]pyrazine-1-carboxamide;
8-(1H-imidazol-1-yl)-N-((1r,4r)-4-((2,2,2-trifluoroethyl)amino)cyclohexyl)-1,7-naphthyridine-6-carboxamide;
8-(thiazol-5-yl)-N-((1r,4r)-4-((2,2,2-trifluoroethyl)amino)cyclohexyl)-1,7-naphthyridine-6-carboxamide;
2-(1H-imidazol-1-yl)-N-(2-(2,2,2-trifluoroethyl)-2-azaspiro[3.5]nonan-7-yl)pyrrolo[2,1-f][1,2,4]triazine-4-carboxamide;
2-(1H-imidazol-1-yl)-N-(6-((2,2,2-trifluoroethyl)amino)spiro[3.3]heptan-2-yl)pyrrolo[2,1-f][1,2,4]triazine-4-carboxamide;
2-(1H-imidazol-1-yl)-N-(6-((2,2,2-trifluoroethyl)amino)spiro[3.3]heptan-2-yl)pyrrolo[2,1-f][1,2,4]triazine-4-carboxamide;
2-(1H-imidazol-1-yl)-N-((1r,3r)-3-((2,2,2-trifluoroethyl)amino)cyclobutyl)pyrrolo[2,1-f][1,2,4]triazine-4-carboxamide;
N-((1r,4r)-4-(3,3-difluoropyrrolidin-1-yl)cyclohexyl)-2-(1H-imidazol-1-yl)pyrrolo[2,1-f][1,2,4]triazine-4-carboxamide;
N-((1S,4r)-4-((S)-3-fluoropyrrolidin-1-yl)cyclohexyl)-2-(1H-imidazol-1-yl)pyrrolo[2,1-f][1,2,4]triazine-4-carboxamide;
N-((1R,4r)-4-((R)-3-fluoropyrrolidin-1-yl)cyclohexyl)-2-(1H-imidazol-1-yl)pyrrolo[2,1-f][1,2,4]triazine-4-carboxamide;
1-(1H-imidazol-1-yl)-N-((1,4r)-4-((2,2,2-trifluoroethyl)amino)cyclohexyl)pyrrolo[1,2-a]pyrazine-3-carboxamide;
4-(1H-imidazol-1-yl)-N-((1r,4r)-4-((2,2,2-trifluoroethyl)amino)cyclohexyl)pyrrolo[2,1-f][1,2,4]triazine-2-carboxamide;
2-(1-methyl-1H-imidazol-5-yl)-N-((1r,4r)-4-((2,2,2-trifluoroethyl)amino)cyclohexyl)pyrrolo[2,1-f][1,2,4]triazine-4-carboxamide;
2-(1-(2-hydroxyethyl)-1H-imidazol-5-yl)-N-((1r,4r)-4-((2,2,2-trifluoroethyl)amino)cyclohexyl)pyrrolo[2,1-f][1,2,4]triazine-4-carboxamide;
8-(1H-imidazol-1-yl)-N-((1r,4r)-4-((2,2,2-trifluoroethyl)amino)cyclohexyl) imidazo[1,2-a]pyrazine-6-carboxamide;
8-(1H-imidazol-1-yl)-N-((1r,4r)-4-((2,2,2-trifluoroethyl)amino)cyclohexyl)-[1,2,4]triazolo[1,5-a]pyrazine-6-carboxamide;
4-(1H-imidazol-1-yl)-N-((1r,4r)-4-((2,2,2-trifluoroethyl)amino)cyclohexyl)pyrazolo[1,5-a]pyrazine-6-carboxamide;
N-((1S,4r)-4-((S)-3-fluoropyrrolidin-1-yl)cyclohexyl)-1-(1H-imidazol-1-yl)pyrrolo[1,2-a]pyrazine-3-carboxamide;
N-((1S,4r)-4-((S)-3-hydroxypyrrolidin-1-yl)cyclohexyl)-2-(1H-imidazol-1-yl)pyrrolo[2,1-f][1,2,4]triazine-4-carboxamide;
N-((1R,4r)-4-((R)-3-fluoropyrrolidin-1-yl)cyclohexyl)-1-(1H-imidazol-1-yl)pyrrolo[1,2-a]pyrazine-3-carboxamide;
N-((1S,4r)-4-((S)-3-fluoropyrrolidin-1-yl)cyclohexyl)-8-(1H-imidazol-1-yl)-[1,2,4]triazolo[1,5-a]pyrazine-6-carboxamide;
N-((1R,4r)-4-((R)-3-fluoropyrrolidin-1-yl)cyclohexyl)-8-(1H-imidazol-1-yl)-[1,2,4]triazolo[1,5-a]pyrazine-6-carboxamide;
N-((1R,4r)-4-((R)-3-hydroxypyrrolidin-1-yl)cyclohexyl)-2-(1H-imidazol-1-yl)pyrrolo[2,1-f][1,2,4]triazine-4-carboxamide;
N-((1R,4r)-4-((R)-3-fluoropyrrolidin-1-yl)cyclohexyl)-4-(1H-imidazol-1-yl)pyrrolo[2,1-f][1,2,4]triazine-2-carboxamide;
2-(1H-imidazol-1-yl)-N-((1r,4r)-4-((2,2,2-trifluoroethyl)amino)cyclohexyl)pyrrolo[2,1-f][1,2,4]triazine-4-carboxamide;
2-(thiazol-5-yl)-N-((1r,4r)-4-((2,2,2-trifluoroethyl)amino)cyclohexyl)pyrrolo[2,1-f][1,2,4]triazine-4-carboxamide;
N-((1r,4r)-4-((2,2-difluoroethyl)amino)cyclohexyl)-2-(1H-imidazol-1-yl)pyrrolo[2,1-f][1,2,4]triazine-4-carboxamide;
N-((1S,4r)-4-((S)-3-fluoropyrrolidin-1-yl)cyclohexyl)-2-(1H-imidazol-1-yl)pyrido[2,3-d]pyrimidine-4-carboxamide;
N-((1r,4r)-4-((2,2-difluoroethyl)amino)cyclohexyl)-2-(1H-imidazol-1-yl)pyrido[2,3-d]pyrimidine-4-carboxamide;
N-((1r,4r)-4-(3,3-difluoropyrrolidin-1-yl)cyclohexyl)-4-(1H-imidazol-1-yl)pyrazolo[1,5-a]pyrazine-6-carboxamide;
N-((1R,4r)-4-((R)-3-fluoropyrrolidin-1-yl)cyclohexyl)-4-(1H-imidazol-1-yl)pyrazolo[1,5-a]pyrazine-6-carboxamide;
N-((1S,4r)-4-((S)-3-fluoropyrrolidin-1-yl)cyclohexyl)-4-(1H-imidazol-1-yl)pyrazolo[1,5-a]pyrazine-6-carboxamide;
N-((1r,4r)-4-((2,2-difluoroethyl)amino)cyclohexyl)-4-(1H-imidazol-1-yl)pyrazolo[1,5-a]pyrazine-6-carboxamide;
N-((1r,4r)-4-((2,2-difluoroethyl)amino)cyclohexyl)-8-(1H-imidazol-1-yl) imidazo[1,2-a]pyrazine-6-carboxamide;
N-((1R,4r)-4-((R)-3-fluoropyrrolidin-1-yl)cyclohexyl)-8-(1H-imidazol-1-yl) imidazo[1,2-a]pyrazine-6-carboxamide;
N-((1S,4r)-4-((S)-3-fluoropyrrolidin-1-yl)cyclohexyl)-8-(1H-imidazol-1-yl) imidazo[1,2-a]pyrazine-6-carboxamide;
N-((1r,4r)-4-(3,3-difluoropyrrolidin-1-yl)cyclohexyl)-8-(1H-imidazol-1-yl) imidazo[1,2-a]pyrazine-6-carboxamide;
2-(5-methyl-1H-imidazol-1-yl)-N-((1r,4r)-4-((2,2,2-trifluoroethyl)amino)cyclohexyl)pyrrolo[2,1-f][1,2,4]triazine-4-carboxamide;
2-(1H-imidazol-1-yl)-N-((1r,4r)-4-(pyrrolidin-1-yl)cyclohexyl)pyrrolo[2,1-f][1,2,4]triazine-4-carboxamide;
2-(1H-imidazol-1-yl)-N-((1s,4s)-4-(pyrrolidin-1-yl)cyclohexyl)pyrrolo[2,1-f][1,2,4]triazine-4-carboxamide;
N-((1r,4r)-4-(3,3-difluoroazetidin-1-yl)cyclohexyl)-8-(1H-imidazol-1-yl)-[1,2,4]triazolo[1,5-a]pyrazine-6-carboxamide;
N-((1r,4r)-4-(3-fluoro-3-methylazetidin-1-yl)cyclohexyl)-8-(1H-imidazol-1-yl)-[1,2,4]triazolo[1,5-a]pyrazine-6-carboxamide;
8-(1H-imidazol-1-yl)-N-((1r,4r)-4-(methyl (2,2,2-trifluoroethyl)amino)cyclohexyl)-[1,2,4]triazolo[1,5-a]pyrazine-6-carboxamide;
N-((1r,4r)-4-(3,3-difluoropyrrolidin-1-yl)cyclohexyl)-8-(1H-imidazol-1-yl)-[1,2,4]triazolo[1,5-a]pyrazine-6-carboxamide;
8-(1H-imidazol-1-yl)-N-((1r,4r)-4-morpholinocyclohexyl)-[1,2,4]triazolo[1,5-a]pyrazine-6-carboxamide;
8-(1H-imidazol-1-yl)-N-(1-(2,2,2-trifluoroethyl) piperidin-4-yl)-[1,2,4]triazolo[1,5-a]pyrazine-6-carboxamide;
N-((1r,4r)-4-(1,1-dioxidothiomorpholino)cyclohexyl)-8-(1H-imidazol-1-yl)-[1,2,4]triazolo[1,5-a]pyrazine-6-carboxamide;
N-((1r,4r)-4-((2,2-difluoroethyl)amino)cyclohexyl)-8-(1H-imidazol-1-yl)-[1,2,4]triazolo[1,5-a]pyrazine-6-carboxamide;
8-(1H-imidazol-1-yl)-N-((1r,4r)-4-((1,1,1-trifluoro-2-methylpropan-2-yl)amino)cyclohexyl)-[1,2,4]triazolo[1,5-a]pyrazine-6-carboxamide;
8-(1H-imidazol-1-yl)-N-((1S,4s)-4-((R)-3-methoxypyrrolidin-1-yl)cyclohexyl)-[1,2,4]triazolo[1,5-a]pyrazine-6-carboxamide;
8-(1H-imidazol-1-yl)-N-((1s,4s)-4-thiomorpholinocyclohexyl)-[1,2,4]triazolo[1,5-a]pyrazine-6-carboxamide;
8-(1H-imidazol-1-yl)-N-((1r,4r)-4-(3-(trifluoromethyl) azetidin-1-yl)cyclohexyl)-[1,2,4]triazolo[1,5-a]pyrazine-6-carboxamide;
N-((1r,4r)-4-((2,2-difluoropropyl)amino)cyclohexyl)-8-(1H-imidazol-1-yl)-[1,2,4]triazolo[1,5-a]pyrazine-6-carboxamide;
N-((1R,4r)-4-((R)-3-fluoropyrrolidin-1-yl)cyclohexyl)-2-(1H-imidazol-1-yl)pyrido[2,3-d]pyrimidine-4-carboxamide;
6-(1H-imidazol-1-yl)-N-((1r,4r)-4-((2,2,2-trifluoroethyl)amino)cyclohexyl)-[1,2,4]triazolo[1,5-a]pyrazine-8-carboxamide;
5-(1H-imidazol-1-yl)-N-((1r,4r)-4-((2,2,2-trifluoroethyl)amino)cyclohexyl)-[1,2,4]triazolo[1,5-c]pyrimidine-7-carboxamide;
   or an enantiomer of any of the foregoing;
   or a pharmaceutically acceptable salt, solvate or prodrug of any of the foregoing.

Preferably the compound of the first or second aspect has a chemical purity of 95% or more, preferably 96% or more, preferably 97% or more, preferably 98% or more, preferably 99% or more, preferably 99.5% or more, preferably 99.8% or more, preferably 99.9% or more, as measured by HPLC or UPLC.

Preferably the compound of the first or second aspect has a stereochemical purity of 95% or more, preferably 96% or more, preferably 97% or more, preferably 98% or more, preferably 99% or more, preferably 99.5% or more, preferably 99.8% or more, preferably 99.9% or more, as measured by XRPD or SFC.

A third aspect of the present invention provides a process for the preparation of a compound, salt, solvate or prodrug according to the first or second aspect of the present invention, wherein the process comprises:
(a) the step of reacting a compound of formula (II) or a salt thereof with an amine of formula (III) or a salt thereof or protected derivative thereof:

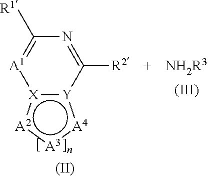

wherein:
   one of R^{1′} and R^{2′} is —C(O) Z and the other one of R^{1′} and R^{2′} is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
   Z is —OH, —OR⁶, —O—CO—R⁶, —F or —Cl;
   R⁶ is C₁-C₃ alkyl; and
   R³, n, A¹, A², A³, A⁴, X and Y are as defined in the first or second aspect of the present invention; or
(b) the step of reacting a compound of formula (IV) or a salt thereof with a compound of formula (V) or a salt thereof:

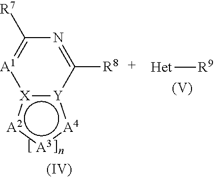

wherein:
   one of R⁷ and R⁸ is —CONHR³ and the other one of R⁷ and R⁸ is a leaving group (such as fluorine, chlorine or bromine);
   Het is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
   R⁹ is a leaving group (such as fluorine, chlorine or bromine), —Sn(C₁-C₄ alkyl)₃ or —B(R¹⁰)₂;
   each R¹⁰ is independently selected from hydroxyl, C₁-C₅ alkoxy and C₁-C₅ alkyl, or two R¹⁰ together with the boron atom to which they are attached form an optionally substituted 5- to 6-membered heterocyclic group; and
   R³, n, A¹, A², A³, A⁴, X and Y are as defined in the first or second aspect of the present invention; or
(c) the step of reacting a compound of formula (VI) or a salt thereof with an amine of formula (III) or a salt thereof or protected derivative thereof:

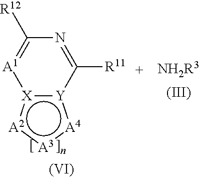

wherein:
   one of R¹¹ and R¹² is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy, and the other one of R¹¹ and R¹² is a leaving group (such as fluorine, chlorine or bromine); and
   R³, n, A¹, A², A³, A⁴, X and Y are as defined in the first or second aspect of the present invention; or
(d) the step of reacting a compound of formula (IV) or a salt thereof with a compound of formula (VII) or a salt thereof:

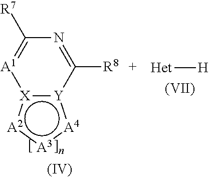

wherein:
   one of R⁷ and R⁸ is —CONHR³ and the other one of R⁷ and R⁸ is a leaving group (such as fluorine, chlorine or bromine);
   Het-H is a heteroaryl compound selected from 1H-pyrrole, 1H-imidazole, 1H-pyrazole, 1H-1,2,3-triazole, 2H-1,2,3-triazole, 1H-1,2,4-triazole and 4H-1,2,4-triazole, wherein the heteroaryl compound is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy; and
   R³, n, A¹, A², A³, A⁴, X and Y are as defined in the first or second aspect of the present invention;
and optionally thereafter carrying out one or more of the following procedures:
   converting a compound of formula (I), (Ia), (Ib), (I′), (Ia′), (Ib′), (I″), (Ia″) or (Ib″) into another compound of formula (I), (Ia), (Ib), (I′), (Ia′), (Ib′), (I″), (Ia″) or (Ib″);
   removing any protecting groups;
   forming a pharmaceutically acceptable salt.

An example of converting a compound of formula (I), (Ia), (Ib), (I′), (Ia′), (Ib′), (I″), (Ia″) or (Ib″) into another compound of formula (I), (Ia), (Ib), (I′), (Ia′), (Ib′), (I″), (Ia″) or (Ib″) is provided in Example 50.

In one embodiment of the process of the present invention, a compound of formula (II) or a salt thereof is reacted with an amine of formula (III) or a salt thereof or protected derivative thereof:

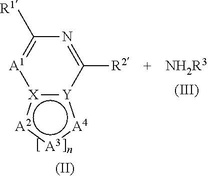

wherein:
one of R^{1′} and R^{2′} is —C(O) Z and the other one of R^{1′} and R^{2′} is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
Z is —OH, —OR⁶, —O—CO—R⁶, —F or —Cl;
R⁶ is C₁-C₃ alkyl; and
R³, n, A¹, A², A³, A⁴, X and Y are as defined in the first or second aspect of the present invention.

When Z is —OH, the compound of formula (II) is a carboxylic acid (IIA). When Z is —OR⁶, the compound of formula (II) is an ester. When Z is —O—CO—R⁶, the compound of formula (II) is an anhydride. When Z is —C₁, the compound of formula (II) is an acid chloride. When Z is —F, the compound of formula (II) is an acid fluoride.

The step of reacting a carboxylic acid (IIA) or a salt thereof with an amine of formula (III) or a salt thereof or protected derivative thereof may be carried out in the presence of uronium-type coupling reagents, such as HATU, or phosphonic anhydrides, such as T3P or T4P, and a base, such as DIPEA or triethylamine. Typically, DMF or DCM is used as a solvent, although other polar aprotic solvents can also be used. Typically, the reaction is carried out at a temperature of about 20-50° C. (typically about 25° C.) and takes about 1-12 hours (typically about 1-6 hours). The salt of the amine of formula (III) may be a hydrochloride salt. The reaction may be carried out under an atmosphere of nitrogen. The amine of formula (III) may be derivatised with a protecting group. The protecting group may be, for example, a tert-butyloxycarbonyl (Boc) group, a fluorenylmethoxycarbonyl (Fmoc) group, an acetamide group, a benzyloxycarbonyl (CBz) group or a para-toluenesulfonamide (Ts) group, although other protecting groups can be used. Conveniently, the amine of formula (III) may be derivatised with a tert-butyloxycarbonyl (Boc) group.

In another embodiment of the process of the present invention, a compound of formula (IV) or a salt thereof is reacted with a compound of formula (V) or a salt thereof:

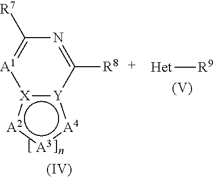

wherein:
one of R⁷ and R⁸ is —CONHR³ and the other one of R⁷ and R⁸ is a leaving group (such as fluorine, chlorine or bromine);
Het is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
R⁹ is a leaving group (such as fluorine, chlorine or bromine), —Sn(C₁-C₄ alkyl) 3 or —B(R¹⁰)₂;
each R¹⁰ is independently selected from hydroxyl, C₁-C₅ alkoxy and C₁-C₅ alkyl, or two R¹⁰ together with the boron atom to which they are attached form an optionally substituted 5- to 6-membered heterocyclic group; and
R³, n, A¹, A², A³, A⁴, X and Y are as defined in the first or second aspect of the present invention.

The compound of formula (V) is a heteroaryl compound activated with, for example, a boron-containing group, a tin-containing group or a leaving group. The activated heteroaryl compound of formula (V) may be used in metal-catalysed cross coupling reactions.

The compound of formula (V) may be a heteroaryl compound activated with a tin-containing group, such as an organotin group. When R⁹ is a group —Sn(C₁-C₄ alkyl)₃, the reaction may conveniently be carried out by a Stille reaction. Typically, R⁹ may be —SnMe₃ or —SnBu₃. The reaction is carried out in the presence of a palladium catalyst, such as Pd(PPh₃)₄, and in a solvent such as dioxane or DMF. The reaction may be carried out in the presence of copper iodide (CuI) and caesium fluoride. Typically, the reaction is carried out at a temperature of about 90-110° C. (typically about 100° C.) for about 10-16 hours (typically about 12 hours). Typically, the reaction is carried out under an atmosphere of nitrogen.

The compound of formula (V) may be a heteroaryl compound activated with a boron-containing group, such as a boronic acid or boronic ester group. When R⁹ is a group —B(R¹⁰)₂, the reaction may conveniently be carried out by a Suzuki reaction. R¹⁰ may be selected such that the heteroaryl compound of formula (V) is activated, for example, by a boronic acid group or a 4,4,5,5-tetramethyl-1,3,2-dioxaborolan-2-yl group.

The activated heteroaryl compound of formula (V) may be, for example, 5-(4,4,5,5-tetramethyl-1,3,2-dioxaborolan-2-yl) thiazole. The reaction is carried out with a palladium catalyst, such as Pd(dppf)Cl₂, in the presence of a base, such as K₂CO₃ or CsCO₃. Typically, the reaction is carried out in a solvent such as dioxane or water or a mixture thereof. Typically, the reaction is carried out at a temperature of about 80-110° C. (typically about 90° C.) for about 1-12 hours. Typically, the reaction is carried out under an atmosphere of nitrogen.

The compound of formula (V) may be a heteroaryl compound activated with a leaving group such as fluorine, chlorine or bromine. Typically, R⁹ is chlorine or bromine. When R⁹ is a leaving group, the compound of formula (V) may be, for example, 5-bromo-1-(2-fluoroethyl)-1H-imidazole, 5-bromo-1-(2,2-difluoroethyl)-1H-imidazole or 5-bromo-1-(oxetan-3-yl)-1H-imidazole.

When R⁹ is a leaving group, step (b) may be carried out by combining a compound of formula (V) or a salt thereof with a compound of formula (IV) or a salt thereof and a diboron compound such as 4,4,4′,4′,5,5,5′,5′-octamethyl-2,2′-bi-1,3,2-dioxaborolane (bis(pinacolato)diboron) in the presence of a palladium catalyst such as palladium (II) acetate or chloro[(di(1-adamantyl)-N-butylphosphine)-2-(2-aminobiphenyl)]palladium (II) (cataCXium-A-Pd-G2).

The reaction is typically carried out in the presence of a base such as caesium fluoride or CsCO₃ and typically in the presence of a ligand such as di (1-adamantyl)-n-butylphosphine. Typically, the reaction is carried out under an atmosphere of nitrogen in a solvent such as toluene, dioxane, methanol or water. Typically, the reaction is carried out in a mixture of dioxane and water or a mixture of methanol and toluene. The reaction is typically carried out at a temperature of about 80-120° C. (typically about 90° C.) for about 6-20 hours (typically about 8-16 hours).

In another embodiment of the process of the present invention, a compound of formula (VI) or a salt thereof is reacted with an amine of formula (III) or a salt thereof or protected derivative thereof:

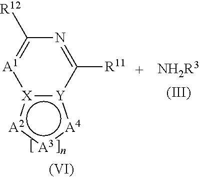

wherein:
one of R¹¹ and R¹² is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy, and the other one of R¹¹ and R¹² is a leaving group (such as fluorine, chlorine or bromine); and
R³, n, A¹, A², A³, A⁴, X and Y are as defined in the first or second aspect of the present invention.

The amine of formula (III) may be derivatised with a protecting group. The protecting group may be, for example, a tert-butyloxycarbonyl (Boc) group, a fluorenylmethoxycarbonyl (Fmoc) group, an acetamide group, a benzyloxycarbonyl (CBz) group or a para-toluenesulfonamide (Ts) group, although other protecting groups can be used. Conveniently, the amine of formula (III) may be derivatised with a tert-butyloxycarbonyl (Boc) group.

The reaction is carried out under an atmosphere of carbon monoxide. The reaction is typically carried out in the presence of a palladium catalyst, such as Pd(dppf)Cl₂, and in the presence of sodium acetate (AcONa) or Na₂CO₃. The reaction may be carried out in the presence of Pd(OAc)₂ with a ligand such as 1,3-bis(diphenylphosphino) propane (DPPP) or 4,5-bis(diphenylphosphino)-9,9-dimethylxanthene (Xantphos). Typically, the reaction is carried out in a solvent such as DMF or toluene. Typically, the reaction is carried out at a temperature of about 70-110° C. (typically about 80-100° C.) for about 12-72 hours.

In another embodiment of the process of the present invention, a compound of formula (IV) or a salt thereof is reacted with a compound of formula (VII) or a salt thereof:

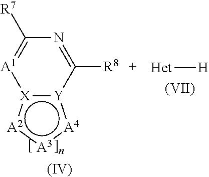

wherein:
one of R⁷ and R⁸ is —CONHR³ and the other one of R⁷ and R⁸ is a leaving group (such as fluorine, chlorine or bromine);
Het-H is a heteroaryl compound selected from 1H-pyrrole, 1H-imidazole, 1H-pyrazole, 1H-1,2,3-triazole, 2H-1,2,3-triazole, 1H-1,2,4-triazole and 4H-1,2,4-triazole, wherein the heteroaryl compound is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy; and
R³, n, A¹, A², A³, A⁴, X and Y are as defined in the first or second aspect of the present invention.

The heteroaryl compound of formula (VII) may be substituted with one or more substituents independently selected from C₁-C₃ alkyl, wherein the C₁-C₃ alkyl is optionally substituted with one or more substituents independently selected from halo, hydroxyl and C₁-C₃ alkoxy. For the avoidance of doubt, said substitution does not replace the hydrogen atom in the N—H bond, such that the heteroaryl compound of formula (VII) has the N—H to act as a nucleophile in step (d).

The reaction is typically carried out in the presence of a base such as DIPEA or triethylamine and in a solvent such as DMF or DMSO. Typically, the reaction is carried out a temperature of about 100-130° C. (typically about 120° C.) for about 5-15 hours (typically about 12 hours).

It will be appreciated by those skilled in the art that in the processes of the present invention certain functional groups such as phenol, hydroxy or amino groups in the reagents may need to be protected by protecting groups. Thus, the preparation of the compounds, salts, solvates and prodrugs of the present invention may involve, at an appropriate stage, the introduction and/or removal of one or more protecting groups.

The protection and deprotection of functional groups are described, for example, in ‘Protective Groups in Organic Chemistry’, edited by J. W. F. McOmie, Plenum Press (1973); ‘Greene's Protective Groups in Organic Synthesis’, 4th edition, T. W. Greene and P. G. M. Wuts, Wiley-Interscience (2007); and ‘Protecting Groups’, 3rd edition, P. J. Kocienski, Thieme (2005).

An example of the introduction and/or removal of one or more protecting groups is provided in Examples 18 and 50.

The compounds of formula (I) may be converted into a pharmaceutically acceptable salt thereof, preferably an acid addition salt such as a formate, hemi-formate, hydrochloride, hydrobromide, benzenesulfonate (besylate), saccharin (e.g. monosaccharin), trifluoroacetate, sulfate, nitrate, phosphate, acetate, fumarate, semi-fumarate, maleate, tartrate, lactate, citrate, pyruvate, succinate, valerate, propanoate, butanoate, malonate, oxalate, 1-hydroxy-2-naphthoate (xinafoate), methanesulfonate or p-toluenesulfonate salt. In one embodiment of the invention, the compounds of formula (I) are in the form of a hydrochloride, formate or fumarate salt.

A salt of a compound of formula (I) may also be formed between a protic acid functionality of a compound of formula (I) and a suitable cation. Suitable cations include, but are not limited to lithium, sodium, potassium, magnesium, calcium and ammonium. In one embodiment of the invention, the salt is a sodium or potassium salt.

Compounds of formula (I) and their salts may be in the form of hydrates or solvates which form another embodiment of the present invention. Such solvates may be formed with common organic solvents including, but not limited to alcoholic solvents e.g. methanol, ethanol or isopropanol.

In one embodiment of the present invention, therapeutically inactive prodrugs are provided. Prodrugs are compounds which, when administered to a subject such as a human, are converted in whole or in part to a compound of formula (I). Generally, the prodrugs are pharmacologically inert chemical derivatives that can be converted in vivo to the active drug molecules to exert a therapeutic effect. Any of the compounds of formula (I) can be administered as a prodrug to increase the activity, bioavailability, or stability of the compound of formula (I) or to otherwise alter the properties of the compound of formula (I). Typical examples of prodrugs include compounds that have biologically labile protecting groups on a functional moiety of the active compound. Prodrugs include, but are not limited to compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

Where the compounds, salts, solvates and prodrugs of the present invention are capable of existing in stereoisomeric forms, it will be understood that the invention encompasses the use of all geometric and optical isomers (including atropisomers) and mixtures thereof. The use of tautomers and mixtures thereof also forms an embodiment of the present invention. The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, more typically less than 1%, and most typically less than 0.5% by weight. Enantiomerically pure isomers are particularly desired.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I. Therefore, the term “hydrogen”, for example, encompasses ¹H, ²H (D) and ³H (T). Similarly, carbon atoms are to be understood to include ¹¹C, ¹²C, ¹³C and ¹⁴C, nitrogen atoms are to be understood to include ¹³N, ¹⁴N and ¹⁵N, oxygen atoms are to be understood to include ¹⁵O, ¹⁶O, ¹⁷O and ¹⁸O, fluorine atoms are to be understood to include ¹⁸F and ¹⁹F, and iodine atoms are to be understood to include ¹²³I, ¹²⁴I, ¹²⁵I, ¹²⁷I and ¹³¹I.

In one embodiment, the compounds, salts, solvates and prodrugs of the present invention may be isotopically labelled. As used herein, an “isotopically labelled” compound is one in which the abundance of a particular nuclide at a particular atomic position within the molecule is increased above the level at which it occurs in nature. Any of the compounds, salts, solvates and prodrugs of the present invention can be isotopically labelled, for example, any of Examples 1-60.

In one embodiment, the compounds, salts, solvates and prodrugs of the present invention may bear one or more radiolabels. Such radiolabels may be introduced by using radiolabel-containing reagents in the synthesis of the compounds, salts, solvates or prodrugs, or may be introduced by coupling the compounds, salts, solvates or prodrugs to chelating moieties capable of binding to a radioactive metal atom. Such radiolabelled versions of compounds, salts, solvates and prodrugs may be used, for example, in diagnostic imaging studies.

In one embodiment, the compounds, salts, solvates and prodrugs of the present invention may be tritiated, i.e. they contain one or more 3H (T) atoms. Any of the compounds, salts, solvates and prodrugs of the present invention can be tritiated, for example, any of Examples 1-60.

The compounds, salts, solvates and prodrugs of the present invention may be amorphous or in a polymorphic form or a mixture of any of these, each of which is an embodiment of the present invention.

The compounds, salts, solvates and prodrugs of the present invention have activity as pharmaceuticals and may be used in treating or preventing a disease, disorder or condition associated with CD38 activity. Diseases, disorders and conditions associated with CD38 activity include:
CNS diseases and diseases requiring treatment via the CNS, including Parkinson's disease (Camacho-Pereira et al, 2016, Cell Metab 23:1127; Perez et al, 2021, Mech Ag & Dev 197:111499; Wakade et al, 2014, PLOS ONE 9: e109818; Yao et al, 2021, npj Parkinsons Dis 7:79); Alzheimer's disease (Blacher et al, 2015, Ann Neurol 78:88; Sonntag et al, 2017, Sci Rep 7:14038); frontotemporal dementia; progressive supranuclear palsy (PSP); tauopathies; other non-Alzheimer's dementias; stroke and ischemic insults (Choe et al, 2011, PLOS ONE 6: e19046); traumatic brain injury (TBI) (Long et al, 2017, Neurochem Res 42:283; Takaso et al, 2020, Sci Rep 10:17795); multiple sclerosis (Herrmann et al, 2016, Dis Mods Mechs 9:1211; Raboon et al, 2019, Front Cell Neurosci 13:258); autoimmune diseases with associated neuronal damage such as Muckle-Wells syndrome; motor neuron disease such as amyotrophic lateral sclerosis (ALS) (Wang et al, 2017, Cell Rep 20:2184); axonal neuropathy and axonal degeneration such as diabetic neuropathy (Lin et al, 2016, Cell Rep 17:69); Wallerian degeneration (Essuman et al, 2017, Neuron 93:1334; Takaso et al, 2020, Sci Rep 10: 17795; Krauss et al, 2020, TiPS 41:281); ataxia telangiectasia, Friedreich's ataxia and other ataxias such as spinocerebellar ataxia 7 (SCA7) (Fang et al, 2016, Cell Metab 24:566);
aging and senescence (Chini et al, 2017, Mol Cell Endocrinol 455:62; Verdin, 2015, Science 350:1208; Imai & Guarente, 2014, Trends Cell Biol 24:464; Schultz & Sinclair, 2016, Cell Metab 23:965);
neuroinflammation (Choe et al, 2011, PLOS ONE 6: e19046; Raboon et al, 2019, Front Cell Neurosci 13:258; Guerreiro et al, 2020, Cells 9:471; Najjar et al, 2013, J Neuroinflamm 10:43);
depression, schizophrenia, anxiety, stress and post-traumatic stress disorder (PTSD) (Tabak et al, 2016, Clin Psychol Sci 4:17);
glaucoma and age-related macular degeneration (AMD) (Cimaglia et al, 2020, Nutrients 12:2871; Jadeja et al, 2020, Oxidative Medicine and Cellular Longevity article 2692794);
hearing loss (Brown et al, 2014, Cell Metab 20:1059; Nakanishi et al, 2020, Frontiers in Neurology 11: article 141; Okur et al, 2020, npj Aging and Mechanisms of Disease 6:1);
autoimmune diseases such as rheumatoid arthritis (RA) and Lupus (Cole et al, 2018, Arthritis Research & Therapy 20:85; Garcia-Rodriguez et al, 2018, Scientific Reports 8:3357);
obesity and metabolic syndrome (Barbosa et al, 2007, FASEB J 21:3629; Chiang et al, 2015, PLOS ONE 10: e0134927).

Therefore, a fourth aspect of the present invention provides a compound, salt, solvate or prodrug according to the first or second aspect of the present invention, for use in therapy, in particular for use in treating or preventing a disease, disorder or condition associated with CD38 activity.

The fourth aspect of the present invention also provides a compound, salt, solvate or prodrug according to the first or second aspect of the present invention, for use in treating or preventing a CNS disease, a disease requiring treatment via the CNS, a neurodegenerative condition, a neurological disease, an age-related disorder, or an inflammatory disorder.

The fourth aspect of the present invention also provides a compound, salt, solvate or prodrug according to the first or second aspect of the present invention, for use in treating or preventing Parkinson's disease; Alzheimer's disease; frontotemporal dementia; progressive supranuclear palsy; a tauopathy; another non-Alzheimer's dementia; stroke; ischemic insult; traumatic brain injury; multiple sclerosis; an autoimmune disease with associated neuronal damage such as Muckle-Wells syndrome; motor neuron disease such as amyotrophic lateral sclerosis; axonal neuropathy or axonal degeneration such as diabetic neuropathy; Wallerian degeneration; ataxia telangiectasia; Friedreich's ataxia; another ataxia such as spinocerebellar ataxia 7; aging; senescence; neuroinflammation; depression; schizophrenia; anxiety; stress; post-traumatic stress disorder; glaucoma; age-related macular degeneration; hearing loss; an autoimmune disease such as rheumatoid arthritis or Lupus; obesity; or metabolic syndrome.

A fifth aspect of the present invention provides a use of a compound, salt, solvate or prodrug according to the first or second aspect of the present invention, for the manufacture of a medicament for treating or preventing a disease, disorder or condition associated with CD38 activity.

The fifth aspect of the present invention also provides a use of a compound, salt, solvate or prodrug according to the first or second aspect of the present invention, for the manufacture of a medicament for treating or preventing a CNS disease, a disease requiring treatment via the CNS, a neurodegenerative condition, a neurological disease, an age-related disorder, or an inflammatory disorder.

The fifth aspect of the present invention also provides a use of a compound, salt, solvate or prodrug according to the first or second aspect of the present invention, for the manufacture of a medicament for treating or preventing Parkinson's disease; Alzheimer's disease; frontotemporal dementia; progressive supranuclear palsy; a tauopathy; another non-Alzheimer's dementia; stroke; ischemic insult; traumatic brain injury; multiple sclerosis; an autoimmune disease with associated neuronal damage such as Muckle-Wells syndrome; motor neuron disease such as amyotrophic lateral sclerosis; axonal neuropathy or axonal degeneration such as diabetic neuropathy; Wallerian degeneration; ataxia telangiectasia; Friedreich's ataxia; another ataxia such as spinocerebellar ataxia 7; aging; senescence; neuroinflammation; depression; schizophrenia; anxiety; stress; post-traumatic stress disorder; glaucoma; age-related macular degeneration; hearing loss; an autoimmune disease such as rheumatoid arthritis or Lupus; obesity; or metabolic syndrome.

A sixth aspect of the present invention provides a method of treating or preventing a disease, disorder or condition associated with CD38 activity; the method comprising administering a therapeutically or prophylactically effective amount of a compound, salt, solvate or prodrug according to the first or second aspect of the present invention, to a patient in need thereof.

The sixth aspect of the present invention also provides a method of treating or preventing a CNS disease, a disease requiring treatment via the CNS, a neurodegenerative condition, a neurological disease, an age-related disorder, or an inflammatory disorder; the method comprising administering a therapeutically or prophylactically effective amount of a compound, salt, solvate or prodrug according to the first or second aspect of the present invention, to a patient in need thereof.

The sixth aspect of the present invention also provides a method of treating or preventing Parkinson's disease; Alzheimer's disease; frontotemporal dementia; progressive supranuclear palsy; a tauopathy; another non-Alzheimer's dementia; stroke; ischemic insult; traumatic brain injury; multiple sclerosis; an autoimmune disease with associated neuronal damage such as Muckle-Wells syndrome; motor neuron disease such as amyotrophic lateral sclerosis; axonal neuropathy or axonal degeneration such as diabetic neuropathy; Wallerian degeneration; ataxia telangiectasia; Friedreich's ataxia; another ataxia such as spinocerebellar ataxia 7; aging; senescence; neuroinflammation; depression; schizophrenia; anxiety; stress; post-traumatic stress disorder; glaucoma; age-related macular degeneration; hearing loss; an autoimmune disease such as rheumatoid arthritis or Lupus; obesity; or metabolic syndrome; the method comprising administering a therapeutically or prophylactically effective amount of a compound, salt, solvate or prodrug according to the first or second aspect of the present invention, to a patient in need thereof.

Unless stated otherwise, in any of the fourth, fifth or sixth aspects of the invention, the subject or patient may be any human or other animal. Typically, the subject or patient is a mammal, more typically a human or a domesticated mammal such as a cow, pig, lamb, sheep, goat, horse, cat, dog, rabbit, mouse etc. Most typically, the subject is a human.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disorder or condition in question. Persons at risk of developing a particular disorder or condition generally include those having a family history of the disorder or condition, or those who have been identified by genetic testing or screening to be particularly susceptible to developing the disorder or condition or those in the prodromal phase of a disorder.

The terms “treat”, “treatment” and “treating” include improvement of the conditions described herein. The terms “treat”, “treatment” and “treating” include all processes providing slowing, interrupting, arresting, controlling, or stopping of the state or progression of the conditions described herein, but does not necessarily indicate a total elimination of all symptoms or a cure of the condition. The terms “treat”, “treatment” and “treating” are intended to include therapeutic as well as prophylactic treatment of such conditions.

For the above-mentioned therapeutic uses the dosage administered will, of course, vary with the compound employed, the mode of administration, the treatment desired and the disorder indicated. For example, the daily dosage of a compound of the invention (that is, a compound of formula (I), (Ia), (Ib), (I′), (Ia′), (Ib′), (I″), (Ia″) or (Ib″), or a pharmaceutically acceptable salt, solvate or prodrug thereof) by oral or parenteral administration may be in the range from 0.01 micrograms per kilogram body weight (μg/kg) to 500 milligrams per kilogram body weight (mg/kg). The desired dosage may be presented at an appropriate interval such as once every other day, once a day, twice a day, three times a day or four times a day.

The compounds of formula (I) and pharmaceutically acceptable salts, solvates and prodrugs thereof may be used on their own, but will generally be administered in the form of a pharmaceutical composition in which the active ingredient is in association with a pharmaceutically acceptable adjuvant, diluent or carrier.

Therefore, a seventh aspect of the present invention provides a pharmaceutical composition comprising a compound, salt, solvate or prodrug according to the first or second aspect of the present invention, in association with a pharmaceutically acceptable adjuvant, diluent or carrier, and optionally one or more other therapeutic agents.

The invention still further provides a process for the preparation of a pharmaceutical composition of the invention which comprises mixing a compound, salt, solvate or prodrug according to the first or second aspect of the present invention, with a pharmaceutically acceptable adjuvant, diluent or carrier.

Conventional procedures for the selection and preparation of suitable pharmaceutical formulations are described in, for example, “Pharmaceutics—The Science of Dosage Form Design”, M. E. Aulton, Churchill Livingstone, 1988.

Pharmaceutically acceptable adjuvants, diluents or carriers that may be used in the pharmaceutical compositions of the invention are those conventionally employed in the field of pharmaceutical formulation, and include, but are not limited to sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, dipotassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the present invention may be administered orally, parenterally, by inhalation spray, rectally, nasally, buccally, vaginally, ocularly, topically or via an implanted reservoir. Oral administration is preferred. The pharmaceutical compositions of the invention may contain any conventional non-toxic pharmaceutically acceptable adjuvants, diluents or carriers. The term parenteral as used herein includes subcutaneous, intracutaneous, intradermal, intravenous, intramuscular, intra-articular, intrasynovial, intrasternal, intrathecal, intralesional, intracranial, intratracheal, intraperitoneal, intraarticular, and epidural injection or infusion techniques. The term topical as used herein includes transdermal, mucosal, sublingual and topical ocular administration.

The pharmaceutical compositions may be in the form of a sterile injectable preparation, for example, as a sterile injectable aqueous or oleaginous suspension. The suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable diluents and solvents that may be employed are mannitol, water, Ringer's solution, and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

The pharmaceutical compositions of this invention may be orally administered in any orally acceptable dosage form including, but not limited to capsules, tablets, caplets, troches, lozenges, powders, granules, and aqueous suspensions, solutions, and dispersions. These dosage forms are prepared according to techniques well-known in the art of pharmaceutical formulation. In the case of tablets for oral use, carriers which are commonly used include lactose, sodium and calcium carbonate, sodium and calcium phosphate, and corn starch. Lubricating agents, such as magnesium stearate, stearic acid or talc, are also typically added. If desired, the tablets may be coated with a material, such as glyceryl monostearate or glyceryl distearate, to delay absorption in the gastrointestinal tract. Tablets may also be effervescent and/or dissolving tablets. For oral administration in capsule form, useful diluents include lactose and dried corn starch. When aqueous suspensions are administered orally, the active ingredient may be combined with emulsifying and suspending agents. If desired, certain sweetening and/or flavouring and/or colouring agents and/or preservatives may be added to any oral dosage form.

The pharmaceutical compositions of the invention may also be administered in the form of suppositories for rectal administration. These compositions can be prepared by mixing the active ingredient with a suitable non-irritating excipient which is solid at room temperature but liquid at the rectal temperature and therefore will melt in the rectum to release the active ingredient. Such materials include, but are not limited to cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels, and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Depending on the mode of administration, the pharmaceutical composition will preferably comprise from 0.05 to 99% by weight, more preferably from 0.05 to 80% by weight, still more preferably from 0.1 to 70% by weight, and even more preferably from 0.1 to 50% by weight of active ingredient, all percentages by weight being based on total composition.

The compounds of the invention may also be administered in conjunction with other compounds used for the treatment of the above conditions.

The invention therefore further relates to combination therapies wherein a compound of the invention or a pharmaceutical composition or formulation comprising a compound of the invention is administered with another therapeutic agent or agents for the treatment of one or more of the conditions previously indicated. The compound of the invention or the pharmaceutical composition or formulation comprising the compound of the invention may be administered simultaneously with, separately from or sequentially to the one or more other therapeutic agents. The compound of the invention and the one or more other therapeutic agents may be comprised in the same pharmaceutical composition or formulation, or in separate pharmaceutical compositions or formulations, i.e. in the form of a kit.

Typically, the mode of administration selected is that most appropriate to the disorder, disease or condition to be treated or prevented. Where one or more further active agents are administered, the mode of administration may be the same as or different to the mode of administration of the compound or pharmaceutical composition of the invention.

Such combination products employ the compounds of this invention within the dosage range described herein and the other pharmaceutically active agent(s) within approved dosage ranges.

### Definitions

An “alkyl” group may be linear (i.e. straight-chained) or branched. Examples of alkyl groups include methyl, ethyl, n-propyl, iso-propyl, n-butyl, iso-butyl, sec-butyl, tert-butyl, n-pentyl, 2-methyl-1-butyl, 3-methyl-1-butyl, 3-methyl-2-butyl, and 2,2-dimethyl-1-propyl groups. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” group is an unsaturated alkyl group having one or more carbon-carbon double bonds. Examples of alkenyl groups include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” group is an unsaturated alkyl group having one or more carbon-carbon triple bonds. Examples of alkynyl groups include ethynyl, propargyl, but-1-ynyl and but-2-ynyl groups. Typically an alkynyl group is a C₂-C₁₂ alkynyl group. More typically an alkynyl group is a C₂-C₆ alkynyl group. An “alkynylene” group is similarly defined as a divalent alkynyl group.

A “cycloalkyl” group (also referred to as a “saturated carbocyclic” group) is a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl group may be monocyclic, bicyclic (e.g. bridged, fused or spiro), or polycyclic.

A “cycloalkenyl” group is a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl group may be monocyclic, bicyclic (e.g. bridged, fused or spiro), or polycyclic.

An “aryl” group is an aromatic hydrocarbyl ring. The term “aryl” includes monocyclic aromatic hydrocarbons (such as phenyl) and polycyclic fused-ring aromatic hydrocarbons (such as naphthyl, anthracenyl and phenanthrenyl). Unless stated otherwise, the term “aryl” does not include “heteroaryl”.

A “heterocyclic” group is a non-aromatic cyclic group which includes one or more carbon atoms and one or more (such as one, two, three or four) heteroatoms, e.g. N, O or S, in the ring structure. A heterocyclic group may be monocyclic, bicyclic (e.g. bridged, fused or spiro), or polycyclic. Typically, a heterocyclic group is a 4- to 14-membered heterocyclic group, which means it contains from 4 to 14 ring atoms. More typically, a heterocyclic group is a 4- to 10-membered heterocyclic group, which means it contains from 4 to 10 ring atoms. Heterocyclic groups include unsaturated heterocyclic groups (such as azetinyl, tetrahydropyridinyl, and 2-oxo-1H-pyridinyl) and saturated heterocyclic groups. Examples of saturated monocyclic heterocyclic groups are azetidinyl, oxetanyl, thietanyl, pyrrolidinyl, tetrahydrofuranyl, tetrahydrothiophenyl, pyrazolidinyl, imidazolidinyl, oxazolidinyl, isoxazolidinyl, dioxolanyl, oxathiolanyl, piperidinyl, tetrahydropyranyl, thianyl, piperazinyl, dioxanyl, morpholinyl and thiomorpholinyl groups. Examples of saturated bicyclic heterocyclic groups are quinuclidinyl, 8-azabicyclo[3.2.1]octanyl, 2-azaspiro[3.3]heptanyl, 6-azaspiro[2.5]octanyl and hexahydro-1H-pyrrolizinyl groups.

A “heteroaryl” group is an aromatic cyclic group which includes one or more carbon atoms and one or more (such as one, two, three or four) heteroatoms, e.g. N, O or S, in the ring structure. Typically, a heteroaryl group is a 5- to 14-membered heteroaryl group, which means it contains from 5 to 14 ring atoms. More typically, a heteroaryl group is a 5- to 10-membered heteroaryl group, which means it contains from 5 to 10 ring atoms. The term “heteroaryl” includes monocyclic aromatic heterocycles (such as pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyridazinyl, pyrimidinyl, pyrazinyl, triazinyl and tetrazinyl) and polycyclic fused-ring aromatic heterocycles (such as indolyl, benzofuranyl, benzothiophenyl, benzoxazolyl, benzisoxazolyl, benzothiazolyl, benzoisothiazolyl, benzimidazolyl, 1H-imidazo[4,5-b]pyridinyl, 1H-imidazo[4,5-c]pyridinyl, quinolinyl, isoquinolinyl, quinoxalinyl, quinazolinyl, phthalazinyl and cinnolinyl). Examples of heteroaryl groups include the following:

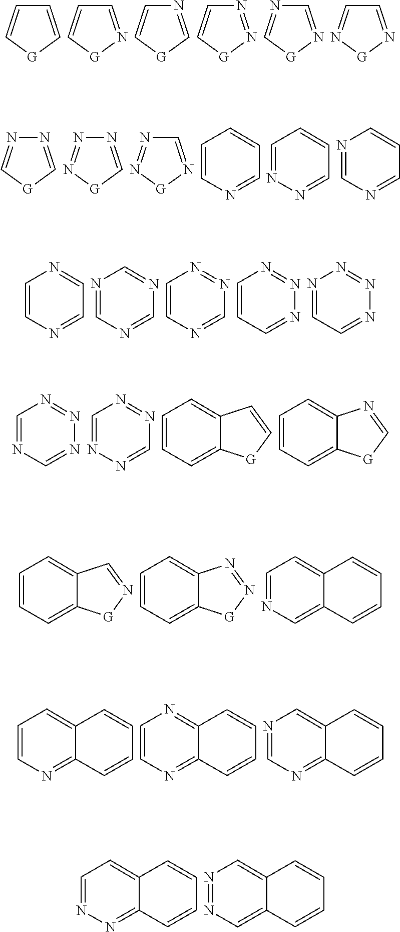

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

The term “halo” includes fluoro, chloro, bromo and iodo. In one embodiment, halo is fluoro.

Unless stated otherwise, where a group is prefixed by the term “halo”, such as a “haloalkyl” or “halomethyl” group, it is to be understood that the group in question is substituted with one or more (such as one, two, three, four or five) halo groups independently selected from fluoro, chloro, bromo and iodo. Typically, the maximum number of halo substituents is limited only by the number of hydrogen atoms available for substitution on the corresponding group without the halo prefix. For example, a “halomethyl” group may contain one, two or three halo substituents. A “haloethyl” or “halophenyl” group may contain one, two, three, four or five halo substituents. Similarly, unless stated otherwise, where a group is prefixed by a specific halo group, it is to be understood that the group in question is substituted with one or more (such as one, two, three, four or five) of the specific halo groups. For example, the term “fluoromethyl” refers to a methyl group substituted with one, two or three fluoro groups, and the term “fluoroethyl” refers to an ethyl group substituted with one, two, three, four or five fluoro groups.

A “hydroxyalkyl” group is an alkyl group substituted with one or more (such as one, two or three) hydroxyl (—OH) groups. Typically a hydroxyalkyl group has one or two hydroxyl substituents, more typically a hydroxyalkyl group has one hydroxyl substituent.

An “alkoxy” group is a —O-alkyl group.

Unless stated otherwise, any reference to an element is to be considered a reference to all isotopes of that element. Thus, for example, unless stated otherwise, any reference to hydrogen is considered to encompass all isotopes of hydrogen including ¹H, ²H (D) and ³H (T). Therefore, for the avoidance of doubt, it is noted that, for example, the terms “alkyl” and “methyl” include, for example, trideuteriomethyl.

Unless stated otherwise, any reference to a compound or group is to be considered a reference to all tautomers of that compound or group.

For the purposes of the present specification, where it is stated that a first atom or group is “directly attached” to a second atom or group it is to be understood that the first atom or group is covalently bonded to the second atom or group with no intervening atom(s) or group(s) being present. So, for example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

When any chemical group or moiety is described as substituted, it will be appreciated that the number and nature of substituents will be selected so as to avoid unstable and sterically undesirable combinations.

### EXAMPLES

The present invention will now be further explained by reference to the following illustrative examples, in which the starting materials and reagents used are available from commercial suppliers or prepared via literature procedures or procedures similar to the ones described in this application.

### Abbreviations

DIPEA N,N-diisopropylethylamine
DMF dimethylformamide
EtOAc ethyl acetate
EtOH ethanol
h hour
HATU 1-[bis(dimethylamino)methylene]-1H-1,2,3-triazolo[4,5-b]pyridinium 3-oxide hexafluorophosphate
HPLC high performance liquid chromatography
MeCN acetonitrile
MeOH methanol
min minutes
Pd₂(dba)₃ tris(dibenzylideneacetone)dipalladium(o)
Pd(dppf)Cl₂ [1,1′-bis(diphenylphosphino) ferrocene]dichloropalladium(II)
RT room temperature
RuPhos Pd G3 (2-dicyclohexylphosphino-2′,6′-diisopropoxy-1,1′-biphenyl) [2-(2′-amino-1,1′-biphenyl)]palladium (II) methanesulfonate
SFC super critical fluid chromatography
T3P 2,4,6-tripropyl-1,3,5,2,4,6-trioxatriphosphinane-2,4,6-trioxide; or propylphosphonic anhydride
TEA triethylamine
TFA trifluoroacetic acid
THF tetrahydrofuran

### General Procedures

Nuclear magnetic resonance (NMR) spectra were recorded at 400 MHz as stated and at 298.2K or 294.1K unless otherwise stated; the chemical shifts (8) are reported in parts per million. Spectra were recorded using a Bruker (trade mark) 400 AVANCE instrument fitted with a 5 mm iprobe or smart probe with instrument controlled by Bruker TopSpin 4.0.9 or Bruker TopSpin 4.1.1 software.

Reactions were monitored using one or more of the following. Agilent 1290 infinity II UPLC coupled with 6130 quadrupole LCMS: mobile phase A: 0.037% TFA in H₂O; mobile phase B: 0.018% CF COOH in CH₃CN; column: Xtimate® C18 2.1*30 mm, 3 μm; column temp.: 50° C.; Sample Temp.: RT; Detection (nm): 220 nm and 254 nm; flow rate: 1.0 mL/min; analysis time: 4.0 min.; measured mass range: 100 to 1500 m/z. Purity was assessed using the following: UPLC with UV (photodiode array) detection over a wide range of wavelengths, normally 220-254 nm, using Shimadzu (trade mark)

Nexera X2 UPLC controlled by Lab Solution software equipped with Acquity UPLC BEH, HSS or HSS T3 C18 columns (2.1 mm id x 50 mm long) operated at 50° C. Mobile phases typically consisted of CH₃CN mixed with H₂O containing either 0.037% TFA. Mass spectra were recorded with a Shimadzu single quadrupole mass spectrometer using DUIS ionisation.

Compounds were purified using Biotage or ISCO® instrument using normal phase chromatography on silica or by preparative high performance liquid chromatography (HPLC).

Preparative HPLC was performed using Gilson GX-281 system using Phenomenex C18 75*30 mm*3 μm; Xtimate C18 100*30 mm*10 μm; Xtimate C18 150*40 mm*10 μm; Xtimate C18 150*40 mm*10 μm; Phenomenex C18 75*30 mm*3 μm or Gemini NX C18 5 μm*10*150 mm columns at RT. Mobile phases typically consisted of CH₃CN mixed with H₂O containing either 0.225% % formic acid or 0.05% ammonia+10 nM NH₄HCO₃, unless otherwise stated.

Super Critical Fluid Chromatography (SFC) chiral analysis were performed on a Waters UPCC with PDA Detector, using a flow rate of 4 mL/min, temperature of RT to 35° C. and a pressure of 1500 psi. Mobile phases typically consisted of supercritical CO₂ and a polar solvent such as CH₃CN, MeOH, EtOH or isopropanol. Column type and eluent are detailed for individual examples. Columns: Chiralpak OD-3 50×4.6 mm, 3 μm; Chiralpak AD-3 50×4.6 mm, 3 μm; Chiral NS-3 100×4.6 mm, 3 μm; Chiral MD-3 100×4.6 mm, 3 μm; Chiralpak IG 50×4.6 mm, 3 μm; (S,S)-Whelk-0-1.8 50×4.6 mm, 1.8 μm; Chiralpak OJ-3 100×4.6 mm, 3 μm.; Detection: 220 nm; sample diluent: CH₃CN, MeOH; injection: 9 μl; isocratic ratio: 5% to 40% of mobile phase.

‘Room temperature’, as used in the present specification, means a temperature in the range from about 18° C. to about 25° C.

### Synthesis of Intermediates

### Intermediate 1:7-(1H-imidazol-1-yl) imidazo[1,2-c]pyrimidine-5-carboxylic acid

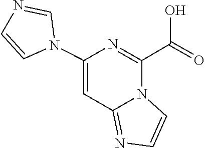 [SMILES: O=C(O)c1nc(-n2ccnc2)cc2nccn12]

Step 1: A mixture of 5,7-dichloroimidazo[1,2-c]pyrimidine (2.00 g, 10.6 mmol), tributyl(1-ethoxyvinyl) stannane (3.84 g, 10.64 mmol) and Pd(PPh₃)₂Cl₂ (746 mg, 1.06 mmol) in DMF (20 ml) was degassed with N₂ (3×) and stirred at 100° C. for 2 h. The mixture was cooled to RT, treated with sat. aq. KF (80 ml) and extracted with EtOAc (3×80 ml). The combined organic layers were washed with brine (80 ml), dried over Na₂SO₄, filtered and concentrated under reduced pressure. Purification of the residue by flash silica gel chromatography (20 g SepaFlash® Silica Flash, 0 to 50% EtOAc in petroleum ether) gave 7-chloro-5-(1-ethoxyvinyl) imidazo[1,2-c]pyrimidine (1.59 g, 67%) as a yellow solid. ¹H NMR (400 MHz, DMSO-d₆): δ 8.23 (s, 1H), 7.84 (s, 1H), 7.74 (d, J=1.5 Hz, 1H), 5.28 (d, J=2.9 Hz, 1H), 4.93 (d, J=2.9 Hz, 1H), 4.07 (q, J=7.0 Hz, 2H), 1.38-1.38 (m, 1H), 1.41 (t, J=6.9 Hz, 2H).

Step 2: A solution of 7-chloro-5-(1-ethoxyvinyl) imidazo[1,2-c]pyrimidine (1.59 g, 7.11 mmol) in dioxane (80 ml) was treated with KMnO₄ (2.02 g, 12.8 mmol), NaIO₄ (3.04 g, 14.22 mmol) and H₂O (12 ml). The mixture was stirred at 30° C. for 2 h. The pH of the mixture was adjusted to 7-8 by the addition of sat. aq. K₂CO₃ solution. The mixture was diluted with H₂O (50 ml) and extracted with EtOAc (3×50 ml). The combined organic layers were washed with brine (50 ml), dried over Na₂SO₄, filtered and concentrated under reduced pressure. Purification of the residue by flash chromatography (12 g SepaFlash® Silica Flash, 0 to 50% EtOAc in petroleum ether) gave ethyl 7-chloroimidazo[1,2-c]pyrimidine-5-carboxylate (790 mg, 49%) as a yellow solid. ¹H NMR (400 MHz, DMSO-d₆): δ 8.67 (s, 1H), 8.16 (s, 1H), 7.89 (d, J=1.4 Hz, 1H), 4.48 (q, J=7.1 Hz, 2H), 1.40 (t, J=7.1 Hz, 3H).

Step 3: A mixture of ethyl 7-chloroimidazo[1,2-c]pyrimidine-5-carboxylate (200 mg, 0.886 mmol), imidazole (121 mg, 1.77 mmol), K₂CO₃ (368 mg, 2.66 mmol) and RuPhos Pd G3 (148.27 mg, 0.177 mmol) in dioxane (1 ml) was degassed with N₂ (3×), and stirred at 100° C. for 16 h. The mixture was cooled to RT, filtered and concentrated under reduced pressure to give 7-(1H-imidazol-1-yl) imidazo[1,2-c]pyrimidine-5-carboxylic acid(180 mg) as a yellow solid, which was used in the next step without further purification.

### Intermediate 2:2-(1H-imidazol-1-yl)pyrido[2,3-d]pyrimidine-4-carboxylic acid

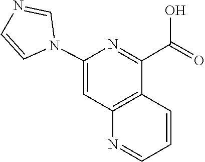 [SMILES: O=C(O)c1nc(-n2ccnc2)cc2ncccc12]

Step 1: A mixture of 2,4-dichloropyrido[2,3-d]pyrimidine (2.00 g, 10.0 mmol), tributyl(1-ethoxyvinyl) stannane (3.61 g, 10.0 mmol) and Pd(PPh₃)₂Cl₂ (702 mg, 1.00 mmol) in DMF (20 ml) was degassed with N₂ (3×) and stirred at 100° C. for 1 h. The mixture was cooled to RT, quenched with sat. aq. KF solution (100 ml) at 25° C. and extracted with EtOAc (3×100 ml). The combined organic layers were washed with brine (300 ml), dried over anhydrous Na₂SO₄, filtered and concentrated under reduced pressure to give 2-chloro-4-(1-ethoxyvinyl)pyrido[2,3-d]pyrimidine (2.30 g, crude) as brown oil, which was used in the next step directly without further purification. ¹H NMR (400 MHz, DMSO-d₆): δ 9.30 (dd, J=1.9, 4.2 Hz, 1H), 8.98 (dd, J=1.9, 8.5 Hz, 1H), 7.80 (dd, J=4.3, 8.5 Hz, 1H), 5.19 (d, J=2.8 Hz, 1H), 4.93 (d, J=2.8 Hz, 1H), 4.10 (q, J=6.9 Hz, 2H), 1.41 (t, J=7.0 Hz, 3H).

Step 2: A solution of 2-chloro-4-(1-ethoxyvinyl)pyrido[2,3-d]pyrimidine (2.3 g, 9.76 mmol) in dioxane (70 ml) was treated with a solution of NaIO₄ (8.35 g, 39.0 mmol) in H₂O (10 ml) and KMnO₄ (1.54 g, 9.76 mmol). The mixture was stirred at 30° C. for 2 h. The pH of the mixture was adjusted by sat. K₂CO₃ (aq.) solution to pH=7-8. The mixture was filtered and the filtrate concentrated under reduced pressure. Purification of the residue by flash chromatography (20 g SepaFlash® Silica Flash, EtOAc/petroleum ether=1:1) gave ethyl 2-chloropyrido[2,3-d]pyrimidine-4-carboxylate (520 mg, 22%) as a yellow solid. ¹H NMR (DMSO-d₆, 400 MHz): δ 9.39 (dd, J=1.9, 4.3 Hz, 1H), 9.03 (dd, J=2.0, 8.5 Hz, 1H), 7.91 (dd, J=4.3, 8.5 Hz, 1H), 4.53 (q, J=7.1 Hz, 2H), 1.41 (t, J=7.1 Hz, 3H).

Step 3: A solution of imidazole (223 mg, 3.28 mmol) and DIPEA (848 mg, 6.56 mmol) in DMF (6 ml) was treated with ethyl 2-chloropyrido[2,3-d]pyrimidine-4-carboxylate (520 mg, 2.19 mmol) and stirred at 100° C. for 2 h. The mixture was concentrated under reduced pressure. Purification of the residue by flash chromatography (4 g SepaFlash® Silica Flash, CH₂Cl₂/MeOH=10:1) gave ethyl 2-(1H-imidazol-1-yl)pyrido[2,3-d]pyrimidine-4-carboxylate (300 mg, 43%) as yellow solid. ¹H NMR (DMSO-d₆, 400 MHz): δ 9.35 (d, J=2.1 Hz, 1H), 8.95 (d, J=7.8 Hz, 1H), 8.72 (s, 1H), 8.09 (s, 1H), 7.82 (dd, J=4.2, 8.4 Hz, 1H), 7.23 (s, 1H), 4.57 (q, J=7.0 Hz, 2H), 1.44 (t, J=7.1 Hz, 3H). MS (ES⁺): 270.0 [M+H]⁺.

Step 4: A mixture of ethyl 2-imidazol-1-ylpyrido[2,3-d]pyrimidine-4-carboxylate (300 mg, 1.11 mmol) and K₂CO₃ (462 mg, 3.34 mmol) in dioxane (2.5 ml) was treated with H₂O (0.5 ml). The mixture was stirred at 70° C. for 2 h and cooled to RT. The pH of the mixture was adjusted to 5 by addition of 1M aq. HCl. The mixture was concentrated to afford 2-(1H-imidazol-1-yl)pyrido[2,3-d]pyrimidine-4-carboxylic acid (720 mg, crude) as yellow solid, which was used in the next step without further purification. ¹H NMR (400 MHz, DMSO-d₆): δ 9.87 (s, 1H), 9.43 (dd, J=1.9, 4.3 Hz, 1H), 9.08 (dd, J=1.9, 8.4 Hz, 1H), 8.50 (s, 1H), 7.92 (dd, J=4.3, 8.4 Hz, 1H), 7.78 (s, 1H).

### Intermediate 3:3-(1H-imidazol-1-yl)pyrrolo[1,2-a]pyrazine-1-carboxylic acid

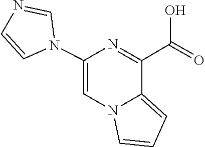 [SMILES: O=C(O)c1nc(-n2ccnc2)cn2cccc12]

Step 1: A solution of methyl 1H-pyrrole-2-carboxylate (10.0 g, 79.9 mmol), 2-chloroacetamide (8.97 g, 95.9 mmol) and Cs₂CO₃ (39.1 g, 119.9 mmol) in DMF (100 ml) was stirred at 25° C. for 2 h, diluted with H₂O (200 ml) and extracted with EtOAc (3×200 ml). The combined organic layers were dried over Na₂SO₄, filtered and concentrated under reduced pressure. Purification of the residue by column chromatography (SiO₂, EtOAc in petroleum=0 to 100%) gave methyl 1-(2-amino-2-oxo-ethyl)pyrrole-2-carboxylate (7.27 g, 38%) as a yellow solid. ¹H NMR (400 MHz, DMSO-d₆): δ 7.38 (s, 1H), 7.12-7.00 (m, 2H), 6.88-6.80 (m, 1H), 6.13-6.06 (m, 1H), 4.89 (s, 2H), 3.69 (s, 2H), 3.71-3.66 (m, 1H).

Step 2: A solution of methyl 1-(2-amino-2-oxo-ethyl)pyrrole-2-carboxylate (5.27 g, 28.9 mmol) in EtOH (200 ml) was treated with 1M potassium tert-butoxide in THF (72.3 ml) under N₂. The mixture was stirred at 70° C. for 12 h, cooled to RT and acidified to pH=4-5 using 2 M aq. HCl. The resulting precipitate was collected by filtration. The cake was washed with H₂O (5 ml) and petroleum ether (10 ml) to give pyrrolo[1,2-a]pyrazine-1,3 (2H,4H)-dione (2.35 g, 52%) as a yellow solid. ¹H NMR (400 MHz, DMSO-d₆): δ 11.30 (s, 1H), 7.21-7.14 (m, 1H), 6.89 (dd, J=1.6, 3.9 Hz, 1H), 6.35 (dd, J=2.6, 3.9 Hz, 1H), 5.00-4.98 (m, 2H).

Step 3: A mixture of 4H-pyrrolo[1,2-a]pyrazine-1,3-dione (2.35 g, 15.6 mmol) and POCl₃ (25 mL) was stirred at 100° C. for 12 h and concentrated under reduced pressure. The residue was quenched with sat. aq. NaHCO₃ (100 ml) and H₂O (50 ml) at 25° C. and extracted with EtOAc (3×200 ml). The combined organic layers were dried over Na₂SO₄, filtered and concentrated under reduced pressure. Purification of the residue by column chromatography (SiO₂, EtOAc in petroleum=0 to 100%) gave 1,3-dichloropyrrolo[1,2-a]pyrazine (585 mg, 16%) as a yellow solid. ¹H NMR (400 MHz, DMSO-d₆): δ 8.67-8.64 (m, 1H), 7.94-7.91 (m, 1H), 7.07-7.00 (m, 2H).

Step 4: A mixture of 1,3-dichloropyrrolo[1,2-a]pyrazine (400 mg, 2.14 mmol), Pd(dppf)Cl₂·CH₂Cl₂ (175 mg, 0.214 mmol) and triethylamine (433 mg, 4.28 mmol) in MeOH (10 ml) was degassed with CO (3×) and stirred at 70° C. for 20 h under CO (50 psi) atmosphere. The mixture was concentrated under reduced pressure to give a residue, which was purified by flash chromatography (4 g SepaFlash® Silica Flash, EtOAc/petroleum ether 1:3) to give methyl 3-chloropyrrolo[1,2-a]pyrazine-1-carboxylate (405 mg, 85%) as yellow solid. ¹H NMR (DMSO-d₆, 400 MHz): δ 8.84 (s, 1H), 7.96 (d, J=1.1 Hz, 1H), 7.34 (d, J=4.1 Hz, 1H), 7.15 (dd, J=2.5, 4.0 Hz, 1H), 3.95 (s, 3H).

Step 5: A mixture of methyl 3-chloropyrrolo[1,2-a]pyrazine-1-carboxylate (140 mg, 0.665 mmol), imidazole (90.5 mg, 1.33 mmol), Cs₂CO₃ (433 mg, 1.33 mmol), Xantphos (76.9 mg, 0.133 mmol) and Pd(OAc)₂ (29.9 mg, 0.033 mmol) in toluene (2 ml) was degassed with N₂ (3×) and stirred at 100° C. for 8 h. The mixture was cooled to RT, filtered and concentrated under reduced pressure to give 3-(1H-imidazol-1-yl)pyrrolo[1,2-a]pyrazine-1-carboxylic acid (170 mg, crude) as yellow solid, which was used in the next step without further purification.

### Intermediate 4:8-(1H-imidazol-1-yl)-1,7-naphthyridine-6-carboxylic acid

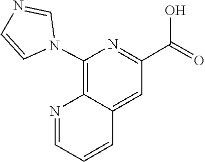 [SMILES: O=C(O)c1cc2cccnc2c(-n2ccnc2)n1]

Step 1: A solution of 8-hydroxy-1,7-naphthyridine-6-carboxylic acid (400 mg, 2.10 mmol) in POCl₃ (6 ml) was stirred at 100° C. for 12 h and concentrated under reduced pressure. The residue was quenched with MeOH (70.6 mg, 2.20 mmol) at 0° C. The mixture was stirred at 0° C. for 1 h and concentrated under reduced pressure. Purification of the residue by flash chromatography (12 g SepaFlash® Silica Flash, EtOAc in petroleum: 0 to 55%) gave methyl 8-chloro-1,7-naphthyridine-6-carboxylate (330 mg, 67%) as a white solid. ¹H NMR (400 MHz, CDCl₃): δ 9.26 (dd, J=1.6, 4.3 Hz, 1H), 8.57 (s, 1H), 8.37 (dd, J=1.6, 8.3 Hz, 1H), 7.81 (dd, J=4.2, 8.3 Hz, 1H), 4.08 (s, 3H).

Step 2: A solution of imidazole (91.7 mg, 1.35 mmol) in THF (6 ml) was treated with NaH (53.9 mg, 1.35 mmol, 60% dispersion in oil) at 0° C., stirred for 30 min and then treated with methyl 8-chloro-1,7-naphthyridine-6-carboxylate (150 mg, 0.674 mmol). The mixture was stirred at 60° C. for 4 h, cooled to RT and quenched by the addition of sat. aq. NH₄Cl solution (10 mL). The mixture was extracted with EtOAc (3×20 ml). The combined organic layers were washed with brine (40 ml), dried over Na₂SO₄, filtered and concentrated under reduced pressure. Purification of the residue by prep. HPLC (Xtimate C18 100*30 mm*10 μm, mobile phase A: 0.225% HCOOH in H₂O, mobile phase B: CH₃CN, 0 to 30% B) and lyophilization gave 8-(1H-imidazol-1-yl)-1,7-naphthyridine-6-carboxylic acid (60 mg, 37%) as a white solid. MS (ES⁺): 241.1 [M+H]⁺.

### Intermediate 5:2-(1H-imidazol-1-yl)pyrrolo[2,1-f][1,2,4]triazine-4-carboxylic acid

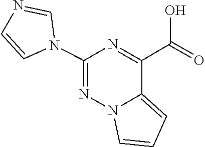 [SMILES: O=C(O)c1nc(-n2ccnc2)nn2cccc12]

Step 1: A mixture of 2,4-dichloropyrrolo[2,1-f][1,2,4]triazine (5.5 g, 29.25 mmol), tributyl(1-ethoxyvinyl) stannane (11.6 g, 32.2 mmol) and Pd(PPh₃)₂Cl₂ (2.05 g, 2.93 mmol) in DMF (60 ml) was degassed with N₂ (3×) and stirred at 100° C. for 2 h. The mixture was cooled to RT, quenched by the addition of sat. aq. KF (100 ml) and extracted with EtOAc (3×100 ml). The combined organic layers were washed with brine (200 ml), dried over Na₂SO₄, filtered and concentrated under reduced pressure. Purification of the residue by flash chromatography (120 g SepaFlash® Silica Flash, EtOAc in petroleum ether 0 to 5%) gave 2-chloro-4-(1-ethoxyvinyl)pyrrolo[2,1-f][1,2,4]triazine (6.00 g, 92%) as an orange solid. ¹H NMR (400 MHz, DMSO-d₆): δ 8.20 (dd, J=1.4, 2.4 Hz, 1H), 7.30 (dd, J=1.4, 4.6 Hz, 1H), 7.08 (dd, J=2.6, 4.6 Hz, 1H), 5.62 (d, J=2.4 Hz, 1H), 4.85 (d, J=2.4 Hz, 1H), 4.01 (q, J=7.0 Hz, 2H), 1.43 (t, J=7.0 Hz, 3H).

Step 2: A solution of 2-chloro-4-(1-ethoxyvinyl)pyrrolo[2,1-f][1,2,4]triazine (6.00 g, 26.8 mmol) in dioxane (250 ml) was treated with a solution of NaIO₄ (11.5 g, 53.7 mmol) and KMnO₄ (847 mg, 5.37 mmol) in H₂O (100 ml) and stirred at 25° C. for 2 h. The mixture was neutralized to pH=7-8 using sat. aq. K₂CO₃ solution and filtered. The filtrate was concentrated under reduced pressure to give a residue, which was purified by flash chromatography (120 g SepaFlash® Silica Flash, EtOAc in petroleum ether 0 to 15%) to give ethyl 2-chloropyrrolo[2,1-f][1,2,4]triazine-4-carboxylate (2.70 g, 45%) as an orange solid. ¹H NMR (400 MHz, DMSO-d₆): δ 8.42 (dd, J=1.4, 2.6 Hz, 1H), 7.44 (dd, J=1.4, 4.8 Hz, 1H), 7.30 (dd, J=2.6, 4.8 Hz, 1H), 4.47 (q, J=7.0 Hz, 2H), 1.39 (t, J=7.0 Hz, 3H).

Step 3: A solution of imidazole (978 mg, 14.4 mmol) in THF (40 ml) was treated in portions with NaH (718 mg, 17.95 mmol, 60% dispersion in oil) at 0° C., stirred for 30 min and then treated with ethyl 2-chloropyrrolo[2,1-f][1,2,4]triazine-4-carboxylate (2.70 g, 11.9 mmol). The mixture was stirred at 60° C. for 4 h, cooled to RT and treated with sat. aq. NH₄Cl solution (50 ml). The mixture was extracted with EtOAc (3×50 ml). The combined organic layers were washed with brine (100 ml), dried over Na₂SO₄, filtered and concentrated under reduced pressure. Purification of the residue by flash chromatography (40 g SepaFlash® Silica Flash, EtOAc in petroleum ether 0 to 75%) gave ethyl 2-imidazol-1-ylpyrrolo[2,1-f][1,2,4]triazine-4-carboxylate (1.40 g, 46%) as an orange solid. ¹H NMR (400 MHz, DMSO-d₆): δ 8.49 (s, 1H), 8.42-8.37 (m, 1H), 7.89 (s, 1H), 7.45 (d, J=4.6 Hz, 1H), 7.27 (dd, J=2.4, 4.6 Hz, 1H), 7.19 (s, 1H), 4.51 (q, J=7.2 Hz, 2H), 1.43 (t, J=7.2 Hz, 3H).

Step 4: A solution of ethyl 2-imidazol-1-ylpyrrolo[2,1-f][1,2,4]triazine-4-carboxylate (1.40 g, 5.44 mmol) in THF (20 ml) was treated with 1 M aq. NaOH solution (10.9 ml) and stirred at 25° C. for 2 h. The mixture was concentrated under reduced pressure and the residue acidified using 1 M aq. HCl solution to pH=5-6. The resulting precipitate was collected by filtration to give 2-(1H-imidazol-1-yl)pyrrolo[2,1-f][1,2,4]triazine-4-carboxylic acid (1.00 g, 80%) as a yellow solid. ¹H NMR (400 MHz, DMSO-d₆): δ 8.58 (s, 1H), 8.35 (dd, J=1.4, 2.4 Hz, 1H), 7.93 (t, J=1.4 Hz, 1H), 7.43 (dd, J=1.2, 4.8 Hz, 1H), 7.22 (dd, J=2.4, 4.8 Hz, 1H), 7.19 (s, 1H).

### Intermediate 6:1-(1H-imidazol-1-yl)pyrrolo[1,2-a]pyrazine-3-carboxylic acid

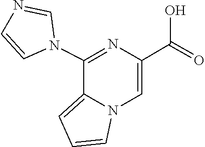 [SMILES: O=C(O)c1cn2cccc2c(-n2ccnc2)n1]

Step 1: A solution of imidazole (96.5 mg, 1.42 mmol) in THF (3 ml) was treated with NaH (113 mg, 2.83 mmol, 60% dispersion in oil) at 0° C. and stirred for 30 min. The mixture was treated with 1,3-dichloropyrrolo[1,2-a]pyrazine (265 mg, 1.42 mmol), stirred at 60° C. for 2.5 h and treated with sat. aq. NH₄Cl solution (5 ml) at 25° C. The mixture was extracted with EtOAc (3×30 ml). The combined organic layers were washed with brine (100 ml), dried over Na₂SO₄, filtered and concentrated under reduced pressure. Purification of the residue by flash chromatography (4 g SepaFlash® Silica Flash, EtOAc/petroleum ether 1:1) gave 3-chloro-1-(1H-imidazol-1-yl)pyrrolo[1,2-a]pyrazine (120 mg, 39%) as a yellow solid. ¹H NMR (DMSO-d₆, 400 MHz): δ 8.64 (d, 1H, J=0.8 Hz), 8.53 (s, 1H), 7.98 (dd, 1H, J=1.1, 2.5 Hz), 7.96 (t, 1H, J=1.3 Hz), 7.28 (d, 1H, J=4.4 Hz), 7.21 (s, 1H), 7.09 (dd, 1H, J=2.6, 4.3 Hz).

Step 2: A mixture of 3-chloro-1-imidazol-1-yl-pyrrolo[1,2-a]pyrazine (80 mg, 0.37 mmol), triethylamine (370 mg, 3.66 mmol), 1,3-bis(diphenylphosphino) propane (75.5 mg, 0.183 mmol) and Pd(OAc)₂ (16.4 mg, 0.073 mmol) in MeOH (5 ml) was degassed with CO (3×). The mixture was stirred at 80° C. for 72 h under CO atmosphere, filtered and concentrated under reduced pressure. Purification of the residue by flash chromatography (4 g SepaFlash® Silica Flash, EtOAc in petroleum ether 0 to 70%) gave methyl 1-imidazol-1-ylpyrrolo[1,2-a]pyrazine-3-carboxylate (25 mg, 28%) as a yellow solid. MS (ES⁺): 243.1 [M+H]⁺.

Step 3: A solution of methyl 1-imidazol-1-ylpyrrolo[1,2-a]pyrazine-3-carboxylate (25 mg, 0.103 mmol) in THF (1 ml) was treated with 1 M aq. LiOH solution (206 μL) and stirred at 25° C. for 1 h. The mixture was acidified by 1 M aq. HCl to pH=5-6 and concentrated under reduced pressure to give 1-(1H-imidazol-1-yl)pyrrolo[1,2-a]pyrazine-3-carboxylic acid (40 mg, crude) as a grey solid. MS (ES⁺): 229.0 [M+H]⁺.

### Intermediate 7:4-(1H-imidazol-1-yl)pyrrolo[2,1-f][1,2,4]triazine-2-carboxylic acid

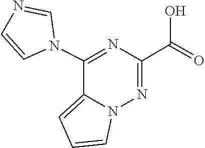 [SMILES: O=C(O)c1nc(-n2ccnc2)c2cccn2n1]

Step 1: A solution of 2,4-dichloropyrrolo[2,1-f][1,2,4]triazine (1.00 g, 5.32 mmol) and imidazole (325 mg, 4.79 mmol) in DMF (10 ml) was treated with DIPEA (2.06 g, 15.9 mmol) and stirred at 25° C. for 2 h. The mixture was diluted with H₂O (30 ml) and extracted with EtOAc (3×50 ml). The combined organic layers were dried over Na₂SO₄, filtered and concentrated under reduced pressure to give a residue, which was purified by flash chromatography (12 g SepaFlash® Silica Flash, EtOAc in petrol ether 0 to 50%) to give 2-chloro-4-(1H-imidazol-1-yl)pyrrolo[2,1-f][1,2,4]triazine (1.03 g, 88%) as a yellow solid. ¹H NMR (400 MHz, DMSO-d₆): δ 8.78 (s, 1H), 8.34 (dd, J=1.2, 2.4 Hz, 1H), 8.13 (t, J=1.4 Hz, 1H), 7.63 (dd, J=1.2, 4.8 Hz, 1H), 7.32-7.25 (m, 1H), 7.18 (dd, J=2.6, 4.8 Hz, 1H).

Step 2: A mixture of 2-chloro-4-imidazol-1-yl-pyrrolo[2,1-f][1,2,4]triazine (50 mg, 0.228 mmol), Pd(dppf)Cl₂·CH₂Cl₂ (18.6 mg, 0.023 mmol) and AcONa (37.4 mg, 0.455 mmol) in DMF/H₂O (1:1, 2 ml) was degassed with CO (3×) and stirred at 80° C. for 48 h under CO (50 psi). The mixture was filtered and the filtrate was evaporated to dryness to give 4-(1H-imidazol-1-yl)pyrrolo[2,1-f][1,2,4]triazine-2-carboxylic acid (80 mg) as a black solid, which was used in the next step without further purification. MS (ES⁺): 230.1 [M+H]⁺.

### Intermediate 8: 2-(1-methyl-1H-imidazol-5-yl)pyrrolo[2,1-f][1,2,4]triazine-4-carboxylic acid

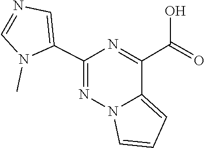 [SMILES: Cn1cncc1-c1nc(C(=O)O)c2cccn2n1]

A mixture of ethyl 2-chloropyrrolo[2,1-f][1,2,4]triazine-4-carboxylate (100 mg, 0.443 mmol), 1-methyl-5-(4,4,5,5-tetramethyl-1,3,2-dioxaborolan-2-yl) imidazole (138 mg, 0.665 mmol), K₂CO₃ (184 mg, 1.33 mmol), Pd(dppf)Cl₂·CH₂Cl₂ (36.2 mg, 0.044 mmol) in dioxane (3 ml) and H₂O (1 ml) was degassed with N₂ (3×) and stirred at 100° C. for 12 h. The mixture was concentrated under reduced pressure to give 2-(1-methyl-1H-imidazol-5-yl)pyrrolo[2,1-f][1,2,4]triazine-4-carboxylic acid (150 mg) as a black solid, which was used in the next step without further purification.

### Intermediate 9: 8-(1H-imidazol-1-yl) imidazo[1,2-a]pyrazine-6-carboxylic acid

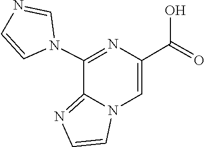 [SMILES: O=C(O)c1cn2ccnc2c(-n2ccnc2)n1]

Step 1: A solution of 6,8-dibromoimidazo[1,2-a]pyrazine (500 mg, 1.81 mmol) and 1H-imidazole (123 mg, 1.81 mmol) in DMF (8 ml) was treated with DIPEA (700 mg, 5.42 mmol) and stirred at 100° C. for 12 h. The mixture was filtered and concentrated under reduced pressure to give a residue, which was purified by flash chromatography (12 g SepaFlash® Silica Flash Column, EtOAc in petroleum ether 0 to 50%) to give 6-bromo-8-(1H-imidazol-1-yl) imidazo[1,2-a]pyrazine (400 mg, 84%) as a yellow solid. ¹H NMR (400 MHz, DMSO-d₆): δ 9.15 (s, 1H), 8.94 (s, 1H), 8.32 (s, 1H), 8.25 (d, J=0.8 Hz, 1H), 7.96 (s, 1H), 7.22 (s, 1H).

Step 2: A mixture of 6-bromo-8-(1H-imidazol-1-yl) imidazo[1,2-a]pyrazine (400 mg, 1.51 mmol), AcONa (373 mg, 4.54 mmol), Pd(dppf)Cl₂·CH₂Cl₂ (123.69 mg, 0.151 mmol) in MeOH (10 ml) was degassed with CO (3×) and stirred at 80° C. for 12 h under CO atmosphere. The mixture was filtered and concentrated under reduced pressure to give a residue, which was purified by flash chromatography (4 g SepaFlash® Silica Flash Column, EtOAc in petroleum ether 0 to 100%) to give methyl 8-(1H-imidazol-1-yl) imidazo[1,2-a]pyrazine-6-carboxylate (60 mg, 16%) as a pink solid. MS (ES⁺): 244.2 [M+H]⁺.

Step 3: A solution of methyl 8-(1H-imidazol-1-yl) imidazo[1,2-a]pyrazine-6-carboxylate (60 mg, 0.247 mmol) in THF (3 ml) was treated with 1 M aq. LiOH solution (0.5 ml) and stirred at 25° C. for 1 h. The mixture was acidified to pH 5-6 using 1M aq. HCl solution and concentrated under reduced pressure to give 8-(1H-imidazol-1-yl) imidazo[1,2-a]pyrazine-6-carboxylic acid (110 mg) as a yellow solid, which was used without further purification. MS (ES⁺): 230 [M+H]⁺.

### Intermediate 10:4-(1H-imidazol-1-yl)pyrazolo[1,5-a]pyrazine-6-carboxylic acid

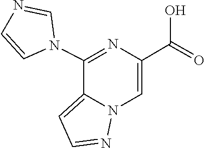 [SMILES: O=C(O)c1cn2nccc2c(-n2ccnc2)n1]

Step 1: A solution of 4,6-dichloropyrazolo[1,5-a]pyrazine (900 mg, 4.79 mmol) in DMF (10 mL) was treated with DIPEA (1.24 g, 9.57 mmol) and 1H-imidazole (326 mg, 4.79 mmol). The mixture was stirred at 100° C. for 2 hours. The reaction mixture was extracted with EtOAc (3×20 mL) and water (30 mL). The combined organic layers were washed with brine (30 mL), dried with anhydrous Na₂SO₄, filtered and concentrated under reduced pressure to give a residue. The residue was purified by flash silica gel chromatography (ISCO®; 20 g SepaFlash® Silica Flash Column, eluent of 0~55% EtOAc in petroleum ether, gradient @50 mL/min) to give 6-chloro-4-(1H-imidazol-1-yl)pyrazolo[1,5-a]pyrazine (880 mg, 4.01 mmol, 84%) as a yellow solid. ¹H NMR (400 MHz, DMSO-d₆) 9.20 (s, 1H), 8.64 (s, 1H), 8.35 (d, J=2.6 Hz, 1H), 8.04 (s, 1H), 7.52 (d, J=2.5 Hz, 1H), 7.25 (s, 1H).

Step 2: A mixture of 6-chloro-4-(1H-imidazol-1-yl)pyrazolo[1,5-a]pyrazine (300 mg, 1.37 mmol), tributyl(1-ethoxyvinyl) stannane (740 mg, 2.05 mmol) and bis(triphenylphosphine) palladium (II) dichloride (95.87 mg, 0.137 mmol) in DMF (3 mL) was degassed and purged with N₂ (3×), and then the mixture was stirred at 100° C. for 1 hour under N₂ atmosphere. The reaction mixture was quenched with sat. KF aq. solution (20 mL) at 25° C., and then extracted with EtOAc (20 mL×3). The combined organic layers were washed with brine (30 mL), dried over anhydrous Na₂SO₄, filtered and concentrated under reduced pressure to give a residue. The residue was purified by column chromatography (SiO₂, EtOAc in petroleum ether 0 to 50%) to give 6-(1-ethoxyvinyl)-4-(1H-imidazol-1-yl)pyrazolo[1,5-a]pyrazine (360 mg, 1.16 mmol, 85% yield, 82% chemical purity) as a white solid. MS ES⁺: 255.7.

Step 3: A solution of 6-(1-ethoxyvinyl)-4-(1H-imidazol-1-yl)pyrazolo[1,5-a]pyrazine (360 mg, 1.41 mmol) in H₂O (3.2 mL) was treated with a solution of NaIO₄ (1.21 g, 5.64 mmol) in dioxane (16 mL) and KMNO₄ (223 mg, 1.41 mmol). The mixture was stirred at 30° C. for 12 hours. The reaction mixture was adjusted with sat. K₂CO₃ aq. solution to pH=7-8, then filtered and concentrated under reduced pressure to give a residue. The residue was purified by column chromatography (SiO₂, EtOAc in petroleum ether 0 to 50%) to give ethyl 4-(1H-imidazol-1-yl)pyrazolo[1,5-a]pyrazine-6-carboxylate (25 mg, 0.097 mmol, 7%) as a white solid. MS ES⁺: 258.0.

Step 4: A solution of ethyl 4-(1H-imidazol-1-yl)pyrazolo[1,5-a]pyrazine-6-carboxylate (25 mg, 0.097 mmol) in tetrahydrofuran (0.2 mL) was treated with 1M LiOH aq. solution (0.486 mL). The mixture was stirred at 25° C. for 1 hour. The reaction mixture was quenched with 1M HCl aq. solution to pH=3-4. The residue was concentrated under reduced pressure to give the crude title compound (60 mg, crude) as a white solid, which was used without further purification. MS ES⁺: 230.0.

### Intermediate 11:2-(5-methyl-1H-imidazol-1-yl)pyrrolo[2,1-f][1,2,4]triazine-4-carboxylic acid

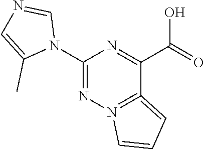

A solution of ethyl 2-chloropyrrolo[2,1-f][1,2,4]triazine-4-carboxylate (100 mg, 0.443 mmol) in DMF (2 mL) was treated with 5-methyl-1H-imidazole (36.4 mg, 0.443 mmol) and KF (51.5 mg, 0.886 mmol). The mixture was stirred at 100° C. for 5 hours. The reaction mixture was concentrated under reduced pressure. The product was purified by prep. HPLC (Column: Welch Xtimate C18 150×30 mm×5 μm, Mobile Phase A: water (NH₄HCO₃), Mobile Phase B: acetonitrile, Flow rate: 25 mL/min, gradient condition from 0% B to 90%). The pure fractions were collected and the volatiles were removed under vacuum. The residue was partitioned between acetonitrile (2 mL) and water (10 mL) and the mixture was lyophilized to dryness to give the title compound (29 mg, 0.119 mmol, 27%) as a yellow solid. ¹H NMR (400 MHz, DMSO-d₆) 8.39-8.34 (m, 1H), 8.06 (dd, J=1.5, 2.4 Hz, 1H), 7.59 (s, 1H), 7.12 (dd, J=1.3, 4.6 Hz, 1H), 6.98 (dd, J=2.5, 4.5 Hz, 1H), 2.21-2.18 (m, 3H).

### Intermediate 12:2-chloropyrrolo[2,1-f][1,2,4]triazine-4-carboxylic acid

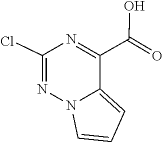 [SMILES: O=C(O)c1nc(Cl)nn2cccc12]

A solution of ethyl 2-chloropyrrolo[2,1-f][1,2,4]triazine-4-carboxylate (300 mg, 1.33 mmol) in THF (2 ml) was treated with NaOH (266 mg, 6.65 mmol) followed by H₂O (1 ml) and stirred at 25° C. for 30 min. The pH of the mixture was adjusted to 5 using 1M aq. HCl solution and concentrated to dryness to give the title compound (350 mg) as brown solid, which was used in the next step directly without further purification. MS (ES⁺): 198.0 [M+H]⁺.

### Intermediate 13: (1r,4r)-4-(3,3-difluoroazetidin-1-yl)cyclohexan-1-amine

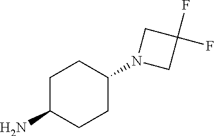 [SMILES: N[C@H]1CC[C@H](N2CC(F)(F)C2)CC1]

Step 1: A solution of 4-(dibenzylamino)cyclohexan-1-one (5 g, 17.0 mmol) and 3,3-difluoroazetidine hydrochloride (2.65 g, 20.5 mmol) in chloroform (30 mL) was treated with AcOH (1.02 g, 17.04 mmol). The mixture was stirred at 25° C. for 1 hour. Sodium triacetoxyborohydride (5.42 g, 25.6 mmol) was then added and the mixture was stirred at 25° C. for 4 hours. 1M aq. NaOH (20 mL) was added, and the mixture extracted with dichloromethane (3×50 mL). The organic layers were dried over anhydrous Na₂SO₄ and concentrated under reduced pressure to give a residue. The residue was purified by flash silica gel chromatography (ISCOR; 40 g SepaFlash® Silica Flash Column, eluent of 8~10% EtOAc in petroleum ether, gradient @50 mL/min) to give N,N-dibenzyl-4-(3,3-difluoroazetidin-1-yl)cyclohexan-1-amine (2.78 g, 4.65 mmol, 27%) as an off-white solid. ¹H NMR (400 MHz, DMSO-d₆) 7.36-7.25 (m, 8H), 7.22-7.16 (m, 2H), 3.56 (s, 4H), 3.47 (t, J=12.4 Hz, 4H), 2.43-2.32 (m, 1H), 2.07-1.95 (m, 1H), 1.84-1.67 (m, 4H), 1.44-1.29 (m, 2H), 0.89-0.71 (m, 2H).

Step 2: A solution of N,N-dibenzyl-4-(3,3-difluoroazetidin-1-yl)cyclohexan-1-amine (2.78 g, 7.50 mmol) in MeOH (30 mL) was treated with Pd(OH) 2 (527 mg, 0.750 mmol, 20% purity). The mixture was then stirred at 50° C. for 12 hours under H₂ atmosphere at 50 psi. The reaction mixture was filtered and the filtrate was washed with MeOH (3×50 mL) and concentrated under reduced pressure to give the title compound (1.25 g, 6.57 mmol, 88%) as a white solid. ¹H NMR (400 MHz, DMSO-d₆) 3.49 (t, J=12.4 Hz, 4H), 2.48-2.39 (m, 1H), 2.00 (br d, J=3.6 Hz, 1H), 1.74-1.59 (m, 4H), 1.07-0.81 (m, 4H).

### Intermediate 14:6-bromo-8-(1H-imidazol-1-yl)-[1,2,4]triazolo[1,5-a]pyrazine

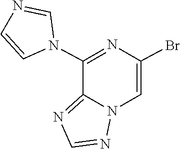 [SMILES: Brc1cn2ncnc2c(-n2ccnc2)n1]

A mixture of 6,8-dibromo-[1,2,4]triazolo[1,5-a]pyrazine (5 g, 18.0 mmol) and 1H-imidazole (1.22 g, 18.0 mmol) in DMF (50 mL) was treated with DIPEA (6.98 g, 54.0 mmol). The mixture was stirred at 100° C. for 1 hour. The reaction mixture was concentrated under reduced pressure to remove the solvent to give a residue. The residue was purified by flash silica gel chromatography (ISCO®; 40 g SepaFlash® Silica Flash Column, eluent of 0~40% EtOAc in petroleum ether, gradient @70 mL/min) to give the title compound as a yellow solid. ¹H NMR (400 MHz, DMSO-d₆) 9.52 (s, 1H), 9.04 (s, 1H), 8.88 (s, 1H), 8.30 (d, J=1.3 Hz, 1H), 7.27 (s, 1H).

### Intermediate 15: (1r,4r)-4-(3-fluoro-3-methylazetidin-1-yl)cyclohexan-1-amine

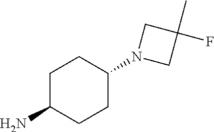

Step 1: A solution of 4-(dibenzylamino)cyclohexan-1-one (2 g, 6.82 mmol) and 3-fluoro-3-methylazetidine hydrochloride (1.03 g, 8.18 mmol) in chloroform (20 mL) was treated with AcOH (409 mg, 6.82 mmol). The mixture was stirred at 25° C. for 1 hour. Then sodium triacetoxyborohydride (2.17 g, 10.2 mmol) was added and the mixture was stirred at 25° C. for 4 hours. 1M NaOH aq. solution (10 mL) was added, and the mixture was then extracted with dichloromethane (3×100 mL). The organic layers were dried over anhydrous Na₂SO₄ and concentrated under reduced pressure to give a residue. The residue was purified by flash silica gel chromatography (ISCO®; 40 g SepaFlash® Silica Flash Column, eluent of 40~50% EtOAc in petroleum ether, gradient @50 mL/min) to give (1r,4r)-N,N-dibenzyl-4-(3-fluoro-3-methylazetidin-1-yl)cyclohexan-1-amine (529.4 mg, 1.39 mmol, 20% yield, 96% chemical purity) as a white solid. ¹H NMR (400 MHz, DMSO-d₆) 7.36-7.24 (m, 8H), 7.22-7.16 (m, 2H), 3.56 (s, 4H), 3.23-3.17 (m, 2H), 3.09-3.00 (m, 2H), 2.39-2.33 (m, 1H), 2.04-1.82 (m, 3H), 1.81-1.69 (m, 4H), 1.51-1.41 (m, 3H), 1.40-1.30 (m, 2H).

Step 2: A mixture of (1r,4r)-N,N-dibenzyl-4-(3-fluoro-3-methylazetidin-1-yl)cyclohexan-1-amine (300 mg, 0.819 mmol), Pd(OH) 2 (287 mg, 0.409 mmol, 20% purity) in MeOH (3 mL) was degassed and purged with H₂ (3×), and then the mixture was stirred at 50° C. for 12 hours under H₂ (50 psi) atmosphere. The reaction mixture was concentrated under reduced pressure to give the title compound (120 mg, 0.644 mmol, 79%) as a white solid, which was used without further purification. MS ES⁺: 187.1.

### Intermediate 16:8-(1H-imidazol-1-yl)-[1,2,4]triazolo[1,5-a]pyrazine-6-carboxylic acid

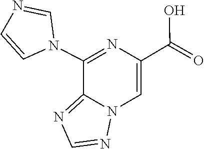 [SMILES: O=C(O)c1cn2ncnc2c(-n2ccnc2)n1]

Step 1: A mixture of 6-bromo-8-(1H-imidazol-1-yl)-[1,2,4]triazolo[1,5-a]pyrazine (Intermediate 14) (3.95 g, 14.9 mmol), AcONa (2.44 g, 29.8 mmol) and Pd(dppf)Cl₂·CH₂Cl₂ (2.43 g, 2.98 mmol) in MeOH (60 mL) was stirred at 80° C. for 16 hours under CO (50 psi) atmosphere. The reaction mixture was concentrated under reduced pressure to give a residue. The residue was purified by flash silica gel chromatography (ISCO®; 40 g SepaFlash® Silica Flash Column, eluent of 0~65% EtOAc in petroleum ether, gradient @60 mL/min) to give methyl 8-(1H-imidazol-1-yl)-[1,2,4]triazolo[1,5-a]pyrazine-6-carboxylate (219 mg, 0.807 mmol, 5.4%) as an orange solid. ¹H NMR (400 MHz, DMSO-d₆) 9.67 (s, 1H), 9.09 (br s, 1H), 9.03 (s, 1H), 8.32 (br s, 1H), 7.29 (br s, 1H), 3.97 (s, 3H).

Step 2: A solution of methyl 8-(1H-imidazol-1-yl)-[1,2,4]triazolo[1,5-a]pyrazine-6-carboxylate (2.9 g, 11.9 mmol) in dioxane: H₂O=5:1 (90 mL) was treated with K₂CO₃ (8.21 g, 59.4 mmol). The mixture was stirred at 70° C. for 12 hours. The reaction mixture was quenched with 1M HCl aq. solution until the pH reached 3-4. The mixture was poured into water (30 mL) and extracted with EtOAc (3×30 mL). The combined organic layers were concentrated to afford the title compound (339 mg, crude) as a brown solid. ¹H NMR (400 MHz, DMSO-d₆) 14.95-13.99 (m, 1H), 9.63 (s, 1H), 9.36-9.27 (m, 1H), 9.04 (s, 1H), 8.45 (s, 1H), 7.41 (br s, 1H).

### Intermediate 17:4-(4-aminocyclohexyl)thiomorpholine 1,1-dioxide hydrochloride

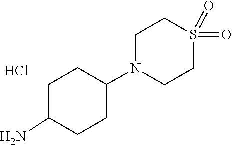 [SMILES: Cl ; NC1CCC(N2CCS(=O)(=O)CC2)CC1]

Step 1: A solution of tert-butyl (4-oxocyclohexyl) carbamate (5 g, 23.4 mmol), thiomorpholine (2.90 g, 28.1 mmol) in chloroform (50 mL) was treated with AcOH (1.41 g, 23.4 mmol). The mixture was stirred at 25° C. for 1 hour. Then sodium triacetoxyborohydride (7.45 g, 35.2 mmol) was added and the mixture was stirred at 25° C. for 2 hours. Sat. NaHCO₃ aq. solution (20 mL) was added, and the reaction mixture extracted with dichloromethane (3×50 mL). The organic layers were dried over anhydrous Na₂SO₄ and concentrated under reduced pressure to give a residue. The residue was purified by flash silica gel chromatography (ISCO®; 40 g SepaFlash® Silica Flash Column, eluent of 0~60% EtOAc in petroleum ether, gradient @50 mL/min) to give tert-butyl (4-thiomorpholinocyclohexyl) carbamate (5.1 g, 16.97 mmol, 72%) as a white solid. ¹H NMR (400 MHz, CDCl₃) 2.86-2.79 (m, 4H), 2.69-2.62 (m, 4H), 2.30 (tt, J=3.3, 11.6 Hz, 1H), 2.11-1.93 (m, 2H), 1.85-1.77 (m, 2H), 1.70-1.63 (m, 2H), 1.59-1.48 (m, 2H), 1.44 (br d, J=4.6 Hz, 9H), 1.39-1.08 (m, 2H).

Step 2: A solution of tert-butyl (4-thiomorpholinocyclohexyl) carbamate (1 g, 3.33 mmol) in dichloromethane (10 mL) was treated with m-CPBA (2.03 g, 9.98 mmol, 85% purity) at 0° C. The mixture was stirred at 25° C. for 12 hours. The mixture was poured into water (30 mL) and extracted with EtOAc (3×30 mL). The combined organic layers were concentrated to afford crude product which was purified by flash silica gel chromatography (ISCO®; 12 g SepaFlash® Silica Flash Column, eluent of 0~70% dichloromethane/MeOH, gradient @40 mL/min) to give tert-butyl (4-(1,1-dioxidothiomorpholino)cyclohexyl) carbamate (742 mg, 2.23 mmol, 67%) as a yellow gum. ¹H NMR (400 MHz, MeOH-d₄) 4.07 (br t, J=12.4 Hz, 2H), 3.75 (br s, 1H), 3.66-3.53 (m, 3H), 3.21 (br d, J=12.9 Hz, 2H), 3.01 (br d, J=14.8 Hz, 2H), 2.44 (br d, J=12.4 Hz, 1H), 2.24 (br d, J=11.9 Hz, 1H), 2.13-1.99 (m, 2H), 1.86-1.64 (m, 4H), 1.46-1.43 (m, 9H), 1.37-1.27 (m, 1H).

Step 3: A solution of tert-butyl (4-(1,1-dioxidothiomorpholino)cyclohexyl) carbamate (100 mg, 0.301 mmol) in dichloromethane (1 mL) was treated with 4M HCl in dioxane (0.075 mL). The mixture was stirred at 25° C. for 0.5 hour. The reaction mixture was concentrated under reduced pressure to give the title compound (70 mg, crude) as a pink solid, which was used without further purification. MS ES⁺: 232.3.

### Intermediate 18: (1r,4r)-N¹-(1,1,1-trifluoro-2-methylpropan-2-yl)cyclohexane-1,4-diamine hydrochloride

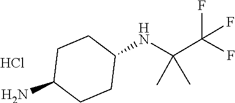

Step 1: A solution of tert-butyl (4-oxocyclohexyl) carbamate (2.17 g, 10.2 mmol) and 1,1,1-trifluoro-2-methylpropan-2-amine hydrochloride (2 g, 12.2 mmol) in dichloromethane (21 mL) was treated with tetraisopropoxytitanium (5.79 g, 20.4 mmol), and after stirring for 12 hours, sodium cyanoborohydride (1.92 g, 30.6 mmol) was added. The mixture was then stirred at 25° C. for a further 4 hours. The reaction mixture was diluted with H₂O (50 mL) and extracted with EtOAc (3×50 mL). The combined organic layers were washed with brine (50 mL), dried over Na₂SO₄, filtered and concentrated under reduced pressure to give a residue. The residue was purified by column chromatography (SiO₂, EtOAc in petroleum ether 0 to 50%) to give tert-butyl ((1r,4r)-4-((1,1,1-trifluoro-2-methylpropan-2-yl)amino)cyclohexyl) carbamate (1.25 g, 3.85 mmol, 38%) as a white solid. ¹H NMR (400 MHz, CDCl₃) 4.33 (br d, J=2.0 Hz, 1H), 3.44-3.32 (m, 1H), 2.90 (br d, J=2.5 Hz, 1H), 1.98 (br d, J=10.9 Hz, 2H), 1.86 (br d, J=10.9 Hz, 2H), 1.46-1.45 (m, 9H), 1.24 (br s, 11H).

Step 2: A solution of tert-butyl ((1r,4r)-4-((1,1,1-trifluoro-2-methylpropan-2-yl)amino)cyclohexyl) carbamate (1.25 g, 3.85 mmol) in dichloromethane (12.5 mL) was treated with 4M HCl in dioxane (49.6 mL). The mixture was stirred at 25° C. for 1 hour. The mixture was concentrated under reduced pressure to give the title compound (1.2 g, crude) as a white solid, which was used without further purification. ¹H NMR (400 MHz, DMSO-d₆) 3.37-3.12 (m, 2H), 2.96 (br d, J=2.5 Hz, 1H), 2.14 (br d, J=11.9 Hz, 2H), 2.03 (br d, J=12.0 Hz, 2H), 1.91-1.89 (m, 1H), 1.77 (br d, J=11.6 Hz, 2H), 1.63 (br s, 6H), 1.48-1.39 (m, 2H), 1.04 (br d, J=6.0 Hz, 1H).

### Intermediate 19: (1S,4s)-4-((R)-3-methoxypyrrolidin-1-yl)cyclohexan-1-amine hydrochloride

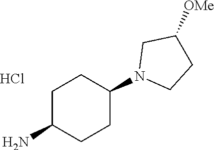 [SMILES: Cl ; CO[C@@H]1CCN([C@H]2CC[C@@H](N)CC2)C1]

Step 1: A mixture of tert-butyl (4-oxocyclohexyl) carbamate (1 g, 4.69 mmol) and (R)-3-methoxypyrrolidine hydrochloride (474 mg, 3.45 mmol) in dichloromethane (10 mL) was treated with AcOH (282 mg, 4.69 mmol). After 1 hour, sodium triacetoxyborohydride (1.49 g, 7.03 mmol) was added in one portion at 25° C. The mixture was stirred at 25° C. for 3 hours. The residue was diluted with H₂O (10 mL) and extracted with EtOAc (3×10 mL). The combined organic layers were dried over Na₂SO₄, filtered and concentrated under reduced pressure to give tert-butyl ((1S,4s)-4-((R)-3-methoxypyrrolidin-1-yl)cyclohexyl) carbamate (1.4 g, crude) as a yellow solid which was used without further purification. MS ES⁺: 298.4.

Step 2: of A mixture tert-butyl ((1S,4s)-4-((R)-3-methoxypyrrolidin-1-yl)cyclohexyl) carbamate (1.61 g, 5.40 mmol) in 4M HCl in dioxane (16.1 mL) was stirred at 25° C. for 1 hour. The reaction mixture was concentrated under reduced pressure to give the title compound (1 g, crude) as a clear oil, which was used without further purification. MS ES⁺: 198.3.

### Intermediate 20: (1r,4r)-4-(3-(trifluoromethyl) azetidin-1-yl)cyclohexan-1-amine

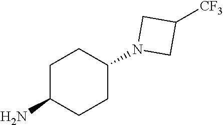 [SMILES: N[C@H]1CC[C@H](N2CC(C(F)(F)F)C2)CC1]

Step 1: A solution of 4-(dibenzylamino)cyclohexan-1-one (750 mg, 2.56 mmol) and 3-(trifluoromethyl) azetidine hydrochloride (496 mg, 3.07 mmol) in dichloromethane (20 mL) was treated with AcOH (154 mg, 2.56 mmol) and sodium triacetoxyborohydride (1.08 g, 5.11 mmol). The mixture was stirred at 25° C. for 12 hours. The reaction mixture was extracted with EtOAc (3×30 mL) and water (20 mL). The combined organic layers were washed with brine (30 mL), dried with anhydrous Na₂SO₄, filtered and concentrated under reduced pressure to give a residue. The residue was purified by flash silica gel chromatography (ISCO®; 12 g SepaFlash® Silica Flash Column, eluent of 0~18% EtOAc in petroleum ether, gradient @40 mL/min) to give (1r,4r)-N,N-dibenzyl-4-(3-(trifluoromethyl) azetidin-1-yl)cyclohexan-1-amine (390 mg, 0.97 mmol, 38%) as a white solid. ¹H NMR (400 MHz, CDCl₃) 7.42-7.20 (m, 10H), 3.69-3.57 (m, 6H), 3.29-3.20 (m, 2H), 2.62-2.47 (m, 1H), 2.20-2.09 (m, 1H), 2.04 (s, 1H), 1.99-1.89 (m, 2H), 1.83 (br d, J=12.4 Hz, 2H), 1.39 (dq, J=3.0, 12.8 Hz, 2H), 1.09-0.90 (m, 2H).

Step 2: A solution of (1r,4r)-N,N-dibenzyl-4-(3-(trifluoromethyl) azetidin-1-yl)cyclohexan-1-amine (390 mg, 0.97 mmol) in EtOH (10 mL) was treated with Pd(OH) 2 (400 mg, 0.80 mmol, 20% purity). The suspension was degassed under vacuum and then purged with H₂ (×3) and then stirred at 50° C. for 12 hours under H₂ atmosphere (50 psi). The reaction mixture was filtered, evaporated to dryness under reduced pressure to give the title compound (160 mg, 0.72 mmol, 74%) as a yellow solid. ¹H NMR (400 MHz, CDCl3) 3.55-3.39 (m, 2H), 3.25-3.07 (m, 3H), 2.49 (br s, 2H), 2.06-1.95 (m, 2H), 1.89 (br d, J=10.9 Hz, 2H), 1.76 (br d, J=12.4 Hz, 2H), 1.20-0.93 (m, 4H).

### Intermediate 21: (1r,4r)-N¹-(2,2-difluoropropyl)cyclohexane-1,4-diamine hydrochloride

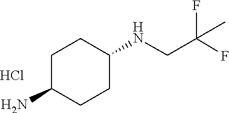

Step 1: A solution of tert-butyl (4-oxocyclohexyl) carbamate (4.05 g, 19.0 mmol) and 2,2-difluoropropan-1-amine hydrochloride (3 g, 22.8 mmol) in dichloromethane (40 mL) was treated with Ti (i-PrO) 4 (10.8 g, 38.0 mmol), and after stirring for 1 hour, sodium cyanoborohydride (3.58 g, 57.0 mmol) was added. The mixture was stirred at 25° C. for a further 12 hours. The mixture was poured into water (100 mL) and extracted with dichloromethane (3×100 mL). The combined organic layers were concentrated to afford crude product which was purified by flash silica gel chromatography (ISCOR; 80 g SepaFlash® Silica Flash Column, eluent of 50~70% EtOAc in petroleum ether, gradient @80 mL/min) to give tert-butyl ((1r,4r)-4-((2,2-difluoropropyl)amino)cyclohexyl) carbamate (3.3 g, 11.29 mmol, 59%) as a white solid.

¹H NMR (400 MHz, DMSO-d₆) 6.79-6.69 (m, 1H), 3.30-3.22 (m, 2H), 3.17 (br s, 1H), 2.72-2.59 (m, 1H), 1.94 (br s, 2H), 1.82-1.75 (m, 2H), 1.66 (brt, J=19.1 Hz, 5H), 1.38-1.38 (m, 2H), 1.37 (s, 9H).

Step 2: A solution of tert-butyl ((1r,4r)-4-((2,2-difluoropropyl)amino)cyclohexyl) carbamate (200 mg, 0.684 mmol) in dichloromethane (2 mL) was treated with 4M HCl in dioxane (2 mL). The mixture was stirred at 25° C. for 1 hour. The reaction mixture was filtered and concentrated under reduced pressure to give the title compound (120 mg, 0.525 mmol, 77%) as a white solid, which was used without further purification. ¹H NMR (400 MHz, DMSO-d₆) 9.64 (s, 1H), 8.33-8.18 (m, 2H), 3.43-3.29 (m, 2H), 3.10-2.96 (m, 1H), 2.23-1.91 (m, 3H), 1.88-1.69 (m, 4H), 1.59-1.30 (m, 5H).

### Intermediate 22:6-(1H-imidazol-1-yl)-[1,2,4]triazolo[1,5-a]pyrazine-8-carboxylic acid

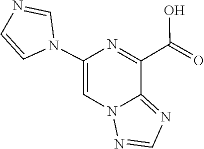 [SMILES: O=C(O)c1nc(-n2ccnc2)cn2ncnc12]

Step 1: A mixture of 6,8-dibromo-[1,2,4]triazolo[1,5-a]pyrazine (4 g, 14.4 mmol), Pd(dppf)Cl₂·CH₂Cl₂ (2.35 g, 2.88 mmol) and AcONa (2.36 g, 28.79 mmol) in EtOH (40 mL) was degassed and purged with CO (3×), and then the mixture was stirred at 80° C. for 12 hours under CO (50 psi) atmosphere. The reaction mixture was filtered and concentrated under reduced pressure to give a residue. The residue was purified by flash silica gel chromatography (ISCO®; 12 g SepaFlash® Silica Flash Column, eluent of 0~15% EtOAc in petroleum ether, gradient @35 mL/min) to give ethyl 6-bromo-[1,2,4]triazolo[1,5-a]pyrazine-8-carboxylate (650 mg, 1.89 mmol, 13% yield, 79% chemical purity) as a white solid. MS ES⁺: 278.7.

Step 2: A mixture of ethyl 6-bromo-[1,2,4]triazolo[1,5-a]pyrazine-8-carboxylate (150 mg, 0.553 mmol), 1H-imidazole (45.2 mg, 0.664 mmol), K₂CO₃ (306 mg, 2.21 mmol), N¹,N²-dimethylethane-1,2-diamine (19.5 mg, 0.221 mmol) and CuI (2.11 mg, 0.011 mmol) in acetonitrile (0.2 mL) was degassed and purged with N₂ (3×), and then the mixture was stirred at 100° C. for 16 hours under N₂ atmosphere under microwave irradiation. The reaction mixture was filtered and concentrated under reduced pressure to give a residue. The product was further purified by prep. HPLC (Column: Xtimate C18 150×40 mm×10 μm, Mobile Phase A: water (NH₄HCO₃), Mobile Phase B: acetonitrile, Flow rate: 25 mL/min, gradient condition from 0% B to 36%). The pure fractions were collected and the volatiles were removed under vacuum. The residue was partitioned between acetonitrile (2 mL) and water (10 mL) and the mixture was lyophilized to dryness to give ethyl 6-(1H-imidazol-1-yl)-[1,2,4]triazolo[1,5-a]pyrazine-8-carboxylate (30 mg, 0.117 mmol, 21%) as a yellow solid. MS ES⁺: 259.0.

Step 3: A solution of ethyl 6-(1H-imidazol-1-yl)-[1,2,4]triazolo[1,5-a]pyrazine-8-carboxylate (30 mg, 0.117 mmol) and THF (0.5 mL) was treated with 1M NaOH aq. solution (0.5 mL). The mixture was stirred at 25° C. for 1 hour. The reaction mixture was quenched with 1M HCl aq. solution to pH 7, and then extracted with EtOAc (3×3 mL). The combined organic layers were dried over Na₂SO₄, filtered and concentrated under reduced pressure to give the title compound (30 mg, crude) as a white solid, which was used without further purification. MS ES⁺: 231.0.

### Intermediate 23:7-chloro-5-(1H-imidazol-1-yl)-[1,2,4]triazolo[1,5-c]pyrimidine

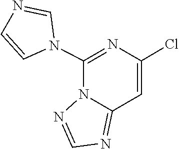 [SMILES: Clc1cc2ncnn2c(-n2ccnc2)n1]

Step 1: To a mixture of 2,4,6-trichloropyrimidine (10 g, 54.5 mmol) in EtOH (100 mL) was added formohydrazide (3.27 g, 54.5 mmol) in one portion at 25° C. The mixture was stirred at 25° C. for 12 hours. The reaction mixture was concentrated under reduced pressure to give a residue which was purified by flash silica gel chromatography (ISCO®; 40 g Sepa Flash® Silica Flash Column, eluent of 0~50% EtOAc in petroleum ether, gradient @60 mL/min) to give N′-(2,6-dichloropyrimidin-4-yl) formohydrazide (2.4 g, 11.59 mmol, 21%) as a white solid. MS ES⁺: 206.9.

Step 2: A mixture of N′-(2,6-dichloropyrimidin-4-yl) formohydrazide (2.4 g, 11.6 mmol) in POCl₃ (24 mL) was heated at 80° C. for 10 hours. The reaction mixture was concentrated under reduced pressure to remove the solvent. The residue was diluted with H₂O (50 mL) and extracted with EtOAc (3×40 mL). The combined organic layers were washed with brine (50 mL), dried over Na₂SO₄, filtered and concentrated under reduced pressure to give crude 5,7-dichloro-[1,2,4]triazolo[4,3-c]pyrimidine (1.7 g, 8.99 mmol, 78%) as a white solid, which was used into the next step without further purification. ¹H NMR (400 MHz, DMSO-d₆) 8.79 (s, 1H), 8.33-8.16 (m, 1H).

Step 3: A mixture of 5,7-dichloro-[1,2,4]triazolo[4,3-c]pyrimidine (160 mg, 0.847 mmol), 1H-imidazole (74.9 mg, 1.10 mmol), methanesulfonato (2-dicyclohexylphosphino-2′,6′-di-i-propoxy-1,1′-biphenyl) (2′-amino-1,1′-biphenyl-2-yl) palladium (II) (142 mg, 0.169 mmol) and Cs₂CO₃ (552 mg, 1.69 mmol) in dioxane (1.5 mL) was degassed and purged with N₂ (3×), and then the mixture was stirred at 100° C. for 2 hours under N₂ atmosphere. The reaction mixture was filtered and concentrated to dryness to give a residue which was purified by prep-TLC (MeOH: dichloromethane=1:20) to give the title compound (25 mg, 0.113 mmol, 13%) as a yellow solid. ¹H NMR (400 MHz, DMSO-d₆) 9.01 (s, 1H), 8.85 (s, 1H), 8.26 (s, 1H), 8.12 (s, 1H), 7.26 (s, 1H).

### Synthesis of Examples

### Example 1: 7-(1H-imidazol-1-yl)-N-(tetrahydro-2H-pyran-4-yl) imidazo[1,2-c]pyrimidine-5-carboxamide

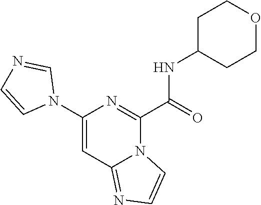 [SMILES: O=C(NC1CCOCC1)c1nc(-n2ccnc2)cc2nccn12]

A solution of 7-(1H-imidazol-1-yl) imidazo[1,2-c]pyrimidine-5-carboxylic acid (Intermediate 1) (180 mg, 0.785 mmol) and tetrahydropyran-4-amine (79.4 mg, 0.785 mmol) in CH₂Cl₂ (2 mL) was treated with triethylamine (636 mg, 3.14 mmol) and T3P (750 mg, 1.18 mmol, 50% in EtOAc). The mixture was stirred at 25° C. for 1 h, diluted with EtOAc (100 ml) and washed with H₂O (3×30 ml) and brine (30 ml). The combined organic layers were dried over Na₂SO₄, filtered and concentrated under reduced pressure. Purification of the residue by prep. HPLC (Welch Xtimate C18 150*30 mm*5 μm, mobile phase A: 0.05% NH₃ and 10 nM NH₄CO₃ in H₂O, mobile phase B: MeOH, 0 to 60% B) and lyophilization gave the title compound (1 mg, 0.4%) as a yellow solid. ¹H NMR (400 MHz, DMSO-d₆): δ 9.14 (d, J=8.5 Hz, 1H), 9.00 (s, 1H), 8.89 (s, 1H), 8.27 (d, J=2.9 Hz, 2H), 7.82 (d, J=1.4 Hz, 1H), 7.18 (s, 1H), 4.19-4.07 (m, 1H), 3.94 (dd, J=2.8, 10.5 Hz, 2H), 3.43 (s, 2H), 1.89-1.77 (m, 4H). MS (ES⁺): 313.1 [M+H]⁺.

### Example 2: 7-(1H-imidazol-1-yl)-N-((1r,4r)-4-((2,2,2-trifluoroethyl)amino)cyclohexyl) imidazo[1,2-c]pyrimidine-5-carboxamide

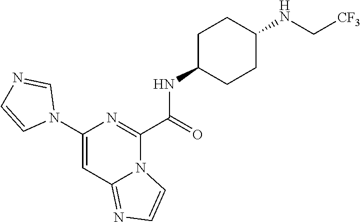 [SMILES: O=C(N[C@H]1CC[C@H](NCC(F)(F)F)CC1)c1nc(-n2ccnc2)cc2nccn12]

A solution of 7-(1H-imidazol-1-yl) imidazo[1,2-c]pyrimidine-5-carboxylic acid (Intermediate 1) (180 mg, 0.785 mmol) and (1r,4r)-N¹-(2,2,2-trifluoroethyl)cyclohexane-1,4-diamine hydrochloride (154 mg, 0.662 mmol) in CH₂Cl₂ (2 ml) was treated with T3P (750 mg, 1.18 mmol, 50% in EtOAc) and triethylamine (397 mg, 3.93 mmol) and stirred at 25° C. for 1 h. The mixture was diluted with CH₂Cl₂ (30 ml), washed with H₂O (3×20 ml) and brine (30 ml). The combined organic layers were dried over Na₂SO₄, filtered and concentrated under reduced pressure. Purification of the residue by prep. HPLC (Phenomenex C18 75*30 mm*3 μm, mobile phase A: 0.05% NH₃ and 10 nM NH₄CO₃ in H₂O, mobile phase B: CH₃CN, 18 to 48% B) and lyophilization gave the title compound (6 mg, 2%) as a light yellow solid. ¹H NMR (400 MHz, DMSO-d₆): δ 9.08-8.96 (m, 2H), 8.88 (s, 1H), 8.26 (d, J=2.4 Hz, 2H), 7.81 (d, J=1.3 Hz, 1H), 7.17 (s, 1H), 3.93-3.78 (m, 1H), 3.29-3.25 (m, 2H), 2.46 (s, 1H), 2.25 (d, J=5.0 Hz, 1H), 1.97 (d, J=11.9 Hz, 2H), 1.86 (d, J=10.8 Hz, 2H), 1.66-1.53 (m, 2H), 1.15 (d, J=10.4 Hz, 2H). MS (ES⁺): 408.0 [M+H]⁺.

### Example 3: 2-(1H-imidazol-1-yl)-N-(tetrahydro-2H-pyran-4-yl)pyrido[2,3-d]pyrimidine-4-carboxamide

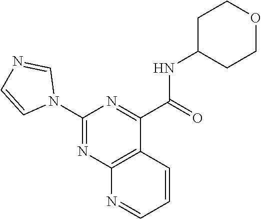 [SMILES: O=C(NC1CCOCC1)c1nc(-n2ccnc2)nc2ncccc12]

A solution of 2-(1H-imidazol-1-yl)pyrido[2,3-d]pyrimidine-4-carboxylic acid (Intermediate 2) (340 mg, 1.41 mmol), tetrahydropyran-4-amine (143 mg, 1.41 mmol) and triethylamine (713 mg, 7.05 mmol) in CH₂Cl₂ (4 mL) was treated with T3P (1.35 g, 2.11 mmol, 50% in EtOAc) and stirred at 25° C. for 1 h. The mixture was treated with H₂O (20 ml) and extracted with CH₂Cl₂ (3×20 ml). The combined organic layers were washed with brine (60 ml), dried over Na₂SO₄, filtered and concentrated under reduced pressure. Purification of the residue by prep. HPLC (Phenomenex C18 75*30 mm*3 μm; mobile phase A: 0.05% NH₃ and 10 nM NH₄CO₃ in H₂O, mobile phase B: CH₃CN, 0 to 40% B) and lyophilization gave the title compound (18 mg, 4%) as an off-white solid. ¹H NMR (400 MHz, DMSO-d₆): δ 9.36-9.30 (m, 2H), 9.18 (d, J=8.3 Hz, 1H), 9.01 (s, 1H), 8.24 (t, J=1.3 Hz, 1H), 7.78 (dd, J=4.3, 8.4 Hz, 1H), 7.24 (s, 1H), 4.22-4.11 (m, 1H), 3.96-3.91 (m, 2H), 3.45 (dt, J=2.3, 11.6 Hz, 2H), 1.86-1.81 (m, 2H), 1.80-1.70 (m, 2H). MS (ES⁺): 325.4 [M+H]⁺.

### Example 4: 2-(1H-imidazol-1-yl)-N-((1r,4r)-4-((2,2,2-trifluoroethyl)amino)cyclohexyl)pyrido[2,3-d]pyrimidine-4-carboxamide

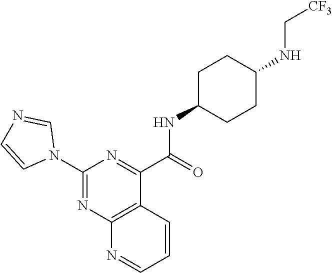 [SMILES: O=C(N[C@H]1CC[C@H](NCC(F)(F)F)CC1)c1nc(-n2ccnc2)nc2ncccc12]

A solution of 2-(1H-imidazol-1-yl)pyrido[2,3-d]pyrimidine-4-carboxylic acid (Intermediate 2) (50 mg, 0.21 mmol), (1r,4r)-N¹-(2,2,2-trifluoroethyl)cyclohexane-1,4-diamine (61 mg, 0.31 mmol) and triethylamine (105 mg, 1.04 mmol) in DMF (1 ml) was treated with HATU (118 mg, 0.311 mmol) and stirred at 25° C. for 1 h. The mixture was treated with H₂O (20 ml) and extracted with EtOAc (2×20 mL). The combined organic layers were washed with brine (60 ml), dried over Na₂SO₄, filtered and concentrated under reduced pressure to give a residue which was purified by prep. HPLC (Xtimate C18 100*30 mm*10 μm; mobile phase A: 0.225% HCOOH in H₂O, mobile Phase B: CH₃CN, 0 to 20% B) and repurified by prep. HPLC (Welch Xtimate C18 150*30 mm*5 μm; mobile phase A: 0.05% NH₃ and 10 nM NH₄CO₃ in H₂O, mobile phase B: CH₃CN, 15 to 55% B). Lyophilization gave the title compound (6 mg, 6%) as an off-white solid. ¹H NMR (400 MHz, DMSO-d₆): δ 9.36-9.29 (m, 2H), 9.06 (d, J=8.3 Hz, 1H), 9.00 (s, 1H), 8.23 (s, 1H), 7.78 (dd, J=4.3, 8.4 Hz, 1H), 7.23 (s, 1H), 3.95-3.80 (m, 1H), 3.29-3.21 (m, 2H), 2.46 (s, 1H), 2.26 (d, J=7.1 Hz, 1H), 1.95 (t, J=15.1 Hz, 4H), 1.58-1.47 (m, 2H), 1.22-1.12 (m, 2H). MS (ES⁺): 420.2 [M+H]⁺.

### Example 5: 3-(thiazol-5-yl)-N-((1r,4r)-4-((2,2,2-trifluoroethyl)amino)cyclohexyl)pyrrolo[1,2-a]pyrazine-1-carboxamide

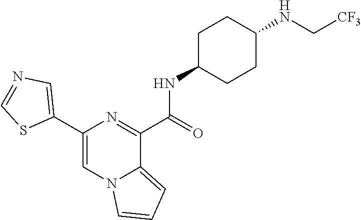 [SMILES: O=C(N[C@H]1CC[C@H](NCC(F)(F)F)CC1)c1nc(-c2cncs2)cn2cccc12]

Step 1: A mixture of methyl 3-chloropyrrolo[1,2-a]pyrazine-1-carboxylate (160 mg, 0.760 mmol), 5-(4,4,5,5-tetramethyl-1,3,2-dioxaborolan-2-yl) thiazole (241 mg, 1.14 mmol), Cs₂CO₃ (743 mg, 2.28 mmol), XPhos (145 mg, 0.304 mmol) in dioxane (1.6 ml) and H₂O (0.4 ml) was treated with Pd₂(dba)₃ (139 mg, 0.152 mmol). The mixture was degassed and purged with N₂ (3×) and stirred at 110° C. for 8 h. The mixture was cooled to RT, filtered and concentrated under reduced pressure to give 3-(thiazol-5-yl)pyrrolo[1,2-a]pyrazine-1-carboxylic acid (200 mg, crude) as brown solid, which was used in the next step without any further purification. MS (ES⁺): 246.0 [M+H]⁺.

Step 2: A solution of 3-(thiazol-5-yl)pyrrolo[1,2-a]pyrazine-1-carboxylic acid (200 mg, 0.815 mmol), (1r,4r)-N¹-(2,2,2-trifluoroethyl)cyclohexane-1,4-diamine (240 mg, 1.22 mmol) and triethylamine (413 mg, 4.08 mmol) in CH₂Cl₂ (5 mL) was treated with T3P (778 mg, 1.22 mmol, 50% in EtOAc) and stirred at 25° C. for 1 h. The mixture was treated with H₂O (20 ml) and extracted with CH₂Cl₂ (3×20 mL). The combined organic layers were washed with brine (60 ml), dried over Na₂SO₄, filtered and concentrated under reduced pressure to give a residue, which was purified by flash chromatography (4 g SepaFlash® Silica Flash, EtOAc in petroleum ether 0 to 100%). The product was further purified by prep. HPLC (Xtimate C18 100*30 mm*10 μm; mobile phase A: 0.225% HCOOH in H₂O, mobile Phase B: CH₃CN, 15 to 35% B) and lyophilized to give the title compound (5 mg, 1%) as a yellow solid. ¹H NMR (400 MHz, DMSO-d₆): δ 9.10 (s, 1H), 9.06 (s, 1H), 8.59 (s, 1H), 8.39 (d, J=8.6 Hz, 1H), 7.89 (s, 1H), 7.43 (d, J=4.0 Hz, 1H), 7.06 (dd, J=2.6, 3.9 Hz, 1H), 3.84-3.75 (m, 1H), 3.25 (d, J=10.4 Hz, 2H), 2.46-2.43 (m, 1H), 2.24 (d, J=5.1 Hz, 1H), 1.95 (d, J=12.1 Hz, 2H), 1.91-1.85 (m, 2H), 1.55-1.45 (m, 2H), 1.19-1.10 (m, 2H). MS (ES⁺): 424.1 [M+H]⁺.

### Example 6: 8-(1H-imidazol-1-yl)-N-((1r,4r)-4-((2,2,2-trifluoroethyl)amino)cyclohexyl)-1,7-naphthyridine-6-carboxamide

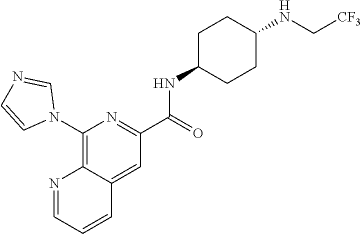 [SMILES: O=C(N[C@H]1CC[C@H](NCC(F)(F)F)CC1)c1cc2cccnc2c(-n2ccnc2)n1]

A solution of 8-(1H-imidazol-1-yl)-1,7-naphthyridine-6-carboxylic acid (Intermediate 4) (40 mg, 0.167 mmol) and (1r,4r)-N¹-(2,2,2-trifluoroethyl)cyclohexane-1,4-diamine (58.1 mg, 250 mmol) in CH₂Cl₂ (1 ml) was treated with T3P (159 mg, 250 mmol, 50% in EtOAc) and triethylamine (84.3 mg, 0.833 mmol) and stirred at 25° C. for 1 h. The mixture was diluted with H₂O (10 ml) and extracted with CH₂Cl₂ (3×10 ml). The combined organic layers were washed with brine (20 ml), dried over Na₂SO₄, filtered and concentrated under reduced pressure. Purification of the residue by prep. HPLC (Phenomenex C18 75*30 mm*3 μm, mobile phase A: 0.05% NH₃ and 10 nM NH₄CO₃ in H₂O, mobile phase B: CH₃CN, 27 to 57% B) and lyophilization gave the title compound (62 mg, 89%) as a white solid. ¹H NMR (400 MHz, DMSO-d₆): δ 9.22 (s, 1H), 9.21 (dd, J=1.8, 4.1 Hz, 1H), 8.75 (dd, J=1.6, 8.5 Hz, 1H), 8.64 (t, J=1.3 Hz, 1H), 8.60-8.53 (m, 2H), 7.95 (dd, J=4.3, 8.4 Hz, 1H), 7.18 (s, 1H), 3.94-3.80 (m, 1H), 3.32-3.20 (m, 2H), 2.46 (dd, J=4.0, 9.5 Hz, 1H), 2.29-2.19 (m, 1H), 1.96 (d, J=11.6 Hz, 2H), 1.85 (d, J=10.3 Hz, 2H), 1.67-1.51 (m, 2H), 1.21-1.07 (m, 2H). MS (ES⁺): 419.2 [M+H]⁺.

### Example 7: 8-(thiazol-5-yl)-N-((1r,4r)-4-((2,2,2-trifluoroethyl)amino)cyclohexyl)-1,7-naphthyridine-6-carboxamide

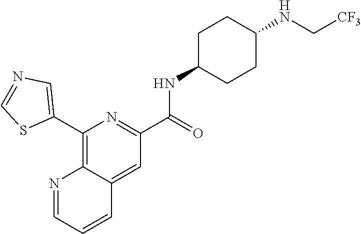 [SMILES: O=C(N[C@H]1CC[C@H](NCC(F)(F)F)CC1)c1cc2cccnc2c(-c2cncs2)n1]

Step 1: A solution of 8-hydroxy-1,7-naphthyridine-6-carboxylic acid (50 mg, 0.263 mmol) in POCl₃ (0.5 ml) was stirred at 90° C. for 12 h and concentrated under reduced pressure to give 8-chloro-1,7-naphthyridine-6-carbonyl chloride (50 mg) as a black solid, which was used in the next step without further purification.

Step 2: A solution of 8-chloro-1,7-naphthyridine-6-carbonyl chloride (50 mg, 0.220 mmol) in CH₂Cl₂ (1 ml) was treated with (1r,4r)-N¹-(2,2,2-trifluoroethyl)cyclohexane-1,4-diamine hydrochloride (51.2 mg, 0.220 mmol) and triethylamine (22.3 mg, 0.220 mmol) and stirred at 0° C. for 30 min. The mixture was diluted with H₂O (10 ml) and washed with CH₂Cl₂ (3×10 ml). The aqueous phase was lyophilized to dryness to give 8-chloro-N-[4-(2,2,2-trifluoroethylamino)cyclohexyl]-1,7-naphthyridine-6-carboxamide (90 mg, crude) as a white solid, which was used in the next step without further purification. MS (ES⁺): 387.0 [M+H]⁺.

Step 3: A solution of 8-chloro-N-[4-(2,2,2-trifluoroethylamino)cyclohexyl]-1,7-naphthyridine-6-carboxamide (90 mg, 0.233 mmol), 5-(4,4,5,5-tetramethyl-1,3,2-dioxaborolan-2-yl) thiazole (49.1 mg, 0.233 mmol), K₂CO₃ (96.5 mg, 0.698 mmol) in dioxane (1 ml) and H₂O (0.2 ml) was treated with Pd(dppf)Cl₂ (17 mg, 0.023 mmol) under N₂. The mixture was stirred at 90° C. for 1 h and concentrated under reduced pressure. The resulting residue was purified by prep. HPLC (Xtimate C18 100*30 mm*10 μm; mobile phase A: 0.223% HCOOH in H₂O, mobile phase B: CH₃CN, 10 to 40% B) and lyophilized to give the title compound (2.5 mg, 2%) as an off-white solid. ¹H NMR (400 MHz, DMSO-d₆): δ 9.55 (s, 1H), 9.32 (s, 1H), 9.23 (dd, J=1.6, 4.1 Hz, 1H), 8.72 (dd, J=1.6, 8.5 Hz, 1H), 8.61 (d, J=8.8 Hz, 1H), 8.59 (s, 1H), 7.93 (dd, J=4.3, 8.4 Hz, 1H), 3.94-3.85 (m, 1H), 3.29-3.23 (m, 3H), 2.46 (s, 1H), 1.97 (d, J=10.6 Hz, 2H), 1.89 (d, J=9.6 Hz, 2H), 1.66-1.57 (m, 2H), 1.20-1.11 (m, 2H). MS (ES⁺): 436.2 [M+H]⁺.

### Example 8: 2-(1H-imidazol-1-yl)-N-(2-(2,2,2-trifluoroethyl)-2-azaspiro[3.5]nonan-7-yl)pyrrolo[2,1-f][1,2,4]triazine-4-carboxamide

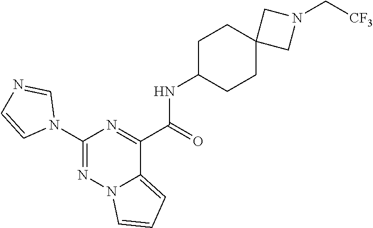 [SMILES: O=C(NC1CCC2(CC1)CN(CC(F)(F)F)C2)c1nc(-n2ccnc2)nn2cccc12]

Step 1: A solution of 2-(1H-imidazol-1-yl)pyrrolo[2,1-f][1,2,4]triazine-4-carboxylic acid (Intermediate 5) (150 mg, 0.654 mmol), tert-butyl 7-amino-2-azaspiro[3.5]nonane-2-carboxylate (236 mg, 0.982 mmol) and triethylamine (199 mg, 1.96 mmol) in CH₂Cl₂ (2 ml) was treated with T3P (625 mg, 0.982 mmol, 50% in EtOAc) and stirred at 25° C. for 1 h. The mixture was treated with H₂O (20 ml) and extracted with CH₂Cl₂ (3×20 mL). The combined organic layers were washed with brine (60 ml), dried over Na₂SO₄, filtered and concentrated under reduced pressure. Purification of the residue by flash chromatography (4 g SepaFlash®) Silica Flash, Eluent of EtOAc in petroleum ether 0 to 100%) gave tert-butyl 7-(2-(1H-imidazol-1-yl)pyrrolo[2,1-f][1,2,4]triazine-4-carboxamido)-2-azaspiro[3.5]nonane-2-carboxylate (310 mg) as yellow solid. Step 2: A solution of tert-butyl 7-(2-(1H-imidazol-1-yl)pyrrolo[2,1-f][1,2,4]triazine-4-carboxamido)-2-azaspiro[3.5]nonane-2-carboxylate (290 mg, 0.642 mmol) in CH₂Cl₂ (3 ml) was treated with CF₃COOH (1.46 g, 12.8 mmol) and stirred at 25° C. for 1 h. The mixture was concentrated under reduced pressure to give N-(2-azaspiro[3.5]nonan-7-yl)-2-imidazol-1-yl-pyrrolo[2,1-f][1,2,4]triazine-4-carboxamide (220 mg, crude) as yellow solid, which was used in the next step without further purification.

Step 3. A solution of N-(2-azaspiro[3.5]nonan-7-yl)-2-imidazol-1-yl-pyrrolo[2,1-f][1,2,4]triazine-4-carboxamide (110 mg, 0.313 mmol) and triethylamine (158 mg, 1.57 mmol) in CH₃CN (2 ml) was treated with 2,2,2-trifluoroethyl trifluoromethanesulfonate (109 mg, 0.470 mmol) and stirred at 70° C. for 10 h. The mixture was concentrated under reduced pressure to give a residue, which was purified by prep. HPLC (Xtimate C18 100*30 mm*10 μm; mobile phase A: 0.223% HCOOH in H₂O, mobile phase B: CH₃CN, 0 to 30% B) and lyophilized to give the title compound (52 mg, 39%) as a yellow solid. ¹H NMR (DMSO-d₆, 400 MHz): δ 9.01 (s, 1H), 8.89 (d, J=8.6 Hz, 1H), 8.31 (dd, J=1.2, 2.4 Hz, 1H), 8.08 (s, 1H), 7.60 (dd, J=1.4, 4.6 Hz, 1H), 7.23 (s, 1H), 7.20 (dd, J=2.4, 4.8 Hz, 1H), 3.85 (d, J=3.8 Hz, 1H), 3.32-3.28 (m, 4H), 3.19 (s, 2H), 1.99 (d, J=6.8 Hz, 2H), 1.75 (s, 2H), 1.58-1.47 (m, 4H). MS (ES⁺): 434.4 [M+H]⁺.

### Example 9 (Isomer 1) and Example 10 (Isomer 2): 2-(1H-imidazol-1-yl)-N-(6-((2,2,2-trifluoroethyl)amino)spiro[3.3]heptan-2-yl)pyrrolo[2,1-f][1,2,4]triazine-4-carboxamide

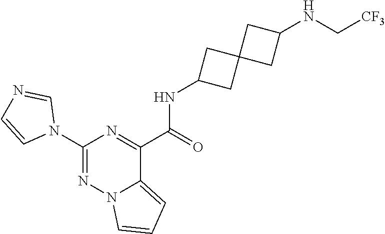 [SMILES: O=C(NC1CC2(CC(NCC(F)(F)F)C2)C1)c1nc(-n2ccnc2)nn2cccc12]

A solution of 2-(1H-imidazol-1-yl)pyrrolo[2,1-f][1,2,4]triazine-4-carboxylic acid (Intermediate 5) (150 mmol) and mg, 0.654 N²-(2,2,2-trifluoroethyl)spiro[3.3]heptane-2,6-diamine hydrochloride (160 mg, 0.654 mmol) in CH₂Cl₂ (3 ml) was treated with T3P (625 mg, 0.982 mmol, 50% in EtOAc) and triethylamine (199 mg, 1.96 mmol) and stirred at 25° C. for 1 h. The mixture was washed with CH₂Cl₂ (3×10 ml) and the aqueous layer was lyophilized to give a residue, which was purified by prep. HPLC (Phenomenex C18 75*30 mm*3 μm, mobile phase A: 0.05% NH₃ and 10 nM NH₄CO₃ in H₂O, mobile phase B: CH₃CN, 32 to 62% B) and lyophilized to give rac-2-(1H-imidazol-1-yl)-N-(6-((2,2,2-trifluoroethyl)amino)spiro[3.3]heptan-2-yl)pyrrolo[2,1-f][1,2,4]triazine-4-carboxamide (85 mg, 31%) as a yellow solid. Separation of the enantiomers by SFC (DAICEL CHIRALPAK AD (250 mm*30 mm, 10 μm; mobile phase: A: supercritical CO₂, mobile phase B: 0.1% NH₃·H₂O in EtOH, A:B=3:2) and lyophilization of the pure fractions gave Isomer 1 (24.5 mg, 9%) and Isomer 2 (23.5 mg, 8%) as a white solids.

Analytical data for Isomer 1 (Example 9): ¹H NMR (400 MHz, DMSO-d₆): δ 9.26 (d, J=8.2 Hz, 1H), 8.98 (s, 1H), 8.30 (d, J=1.6 Hz, 1H), 8.07 (s, 1H), 7.58 (d, J=4.2 Hz, 1H), 7.24-7.14 (m, 2H), 4.49-4.35 (m, 1H), 3.23-3.08 (m, 3H), 2.59-2.52 (m, 1H), 2.41-2.34 (m, 2H), 2.32-2.14 (m, 4H), 1.84-1.74 (m, 2H). MS (ES⁺): 420.2 [M+H]⁺. SFC: Rt=2.209 min, 100%.

Analytical data for Isomer 2 (Example 10): ¹H NMR (400 MHz, DMSO-d₆): δ (9.31 (d, J=8.2 Hz, 1H), 9.03 (s, 1H), 8.36 (dd, J=1.4, 2.4 Hz, 1H), 8.12 (s, 1H), 7.64 (dd, J=1.4, 4.8 Hz, 1H), 7.28-7.22 (m, 2H), 4.55-4.41 (m, 1H), 3.28-3.11 (m, 3H), 2.65-2.57 (m, 1H), 2.47-2.39 (m, 2H), 2.37-2.20 (m, 4H), 1.84 (ddd, J=8.6, 10.8, 12.8 Hz, 2H). MS (ES⁺): 420.2 [M+H]⁺. SFC: Rₜ=2.598 min, 100%.

### Example 11: 2-(1H-imidazol-1-yl)-N-((1r,3r)-3-((2,2,2-trifluoroethyl)amino)cyclobutyl)pyrrolo[2,1-f][1,2,4]triazine-4-carboxamide

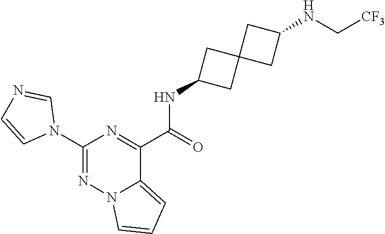 [SMILES: O=C(N[C@H]1CC2(C[C@H](NCC(F)(F)F)C2)C1)c1nc(-n2ccnc2)nn2cccc12]

Step 1: A solution of tert-butyl N-(3-aminocyclobutyl) carbamate (200 mg, 1.07 mmol) and triethylamine (543 mg, 5.37 mmol) in CH₃CN (2 ml) was treated with 2,2,2-trifluoroethyl trifluoromethanesulfonate (299 mg, 1.29 mmol) at 0° C. The mixture was stirred at 70° C. for 12 h and concentrated under reduced pressure to give a residue, which was purified by flash chromatography (4 g SepaFlash® Silica Flash, EtOAc in petrol ether 0 to 40%) to give tert-butyl N-[3-(2,2,2-trifluoroethylamino)cyclobutyl]carbamate (184 mg, 64%) as a white solid. ¹H NMR (400 MHz, DMSO-d₆): δ 7.12 (d, J=7.0 Hz, 1H), 4.02-3.90 (m, 1H), 3.31-3.23 (m, 1H), 3.11 (dq, J=7.4, 10.2 Hz, 2H), 2.65-2.57 (m, 1H), 2.07-1.87 (m, 4H), 1.37 (s, 9H).

Step 2: A solution of tert-butyl N-[3-(2,2,2-trifluoroethylamino)cyclobutyl]carbamate (180 mg, 0.671 mmol) in CH₂Cl₂ (3 ml) was treated with 4 M HCl in dioxane (3 ml) and stirred at 25° C. for 12 h. The mixture was concentrated under reduced pressure to give (1r,3r)-N¹-(2,2,2-trifluoroethyl)cyclobutane-1,3-diamine hydrochloride (163 mg, crude) as a white solid, which was used in the next step without further purification. ¹H NMR (400 MHz, DMSO-d₆): δ 4.02-3.86 (m, 4H), 2.67 (d, J=5.6 Hz, 2H), 2.49-2.42 (m, 2H).

Step 3: A solution of (1r,3r)-N¹-(2,2,2-trifluoroethyl)cyclobutane-1,3-diamine hydrochloride (50 mg, 0.297 mmol) and 2-(1H-imidazol-1-yl)pyrrolo[2,1-f][1,2,4]triazine-4-carboxylic acid (Intermediate 5) (102 mg, 0.446 mmol) in CH₂Cl₂ (1 ml) was treated with DIPEA (115 mg, 0.892 mmol) and T3P (284 mg, 0.446 mmol, 50% in EtOAc) and stirred at 25° C. for 1 h. The mixture was concentrated under reduced pressure to give a residue, which was purified by prep. HPLC (Phenomenex C18 75*30 mm*3 μm, mobile phase A: 0.05% NH₃ and 10 nM NH₄CO₃ in H₂O, mobile phase B: CH₃CN, 23 to 52% B) and lyophilized to give the title compound (3 mg, 3%) as a yellow solid. ¹H NMR (400 MHz, DMSO-d₆): δ 9.32 (d, J=7.6 Hz, 1H), 8.98 (s, 1H), 8.31 (dd, J=1.2, 2.2 Hz, 1H), 8.07 (t, J=1.2 Hz, 1H), 7.58 (dd, J=1.2, 4.8 Hz, 1H), 7.26-7.13 (m, 2H), 4.67-4.55 (m, 1H), 3.43 (d, J=3.8 Hz, 1H), 3.23-3.14 (m, 2H), 2.86-2.77 (m, 1H), 2.44-2.35 (m, 2H), 2.15 (ddd, J=3.6, 8.4, 12.4 Hz, 2H). MS (ES⁺): 380.1 [M+H]⁺.

### Example 12: N-((1r,4r)-4-(3,3-difluoropyrrolidin-1-yl)cyclohexyl)-2-(1H-imidazol-1-yl)pyrrolo[2,1-f][1,2,4]triazine-4-carboxamide

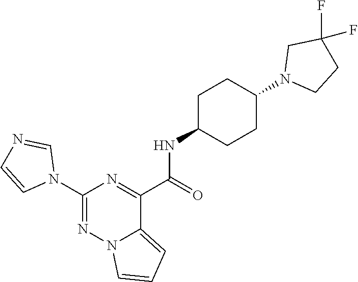 [SMILES: O=C(N[C@H]1CC[C@H](N2CCC(F)(F)C2)CC1)c1nc(-n2ccnc2)nn2cccc12]

Step 1: A solution of 4-(dibenzylamino)cyclohexanone (0.50 g, 1.70 mmol) and 3,3-difluoropyrrolidine hydrochloride (269 mg, 1.87 mmol) in CH₂Cl₂ (25 ml) was treated with AcOH (102 mg, 1.70 mmol), stirred at 25° C. for 1 h, cooled to o° C., treated with NaBH₃CN (214 mg, 3.41 mmol) in portions, warmed to 25° C. and stirred for 3 h. The mixture was quenched with 1 M aq. NaOH solution (75 ml) and extracted with CH₂Cl₂ (3×75 ml). The organic layers were washed with sat. aq. NaCl solution (75 ml) and dried over Na₂SO₄, filtered and concentrated under reduced pressure. Purification of the residue by prep. HPLC (Xtimate C18 150*40 mm*10 μm; mobile phase A: 0.05% NH₃ and 10 nM NH₄CO₃ in H₂O, mobile phase B: CH₃CN, 55 to 95% B) and lyophilization gave

N,N-dibenzyl-4-(3,3-difluoropyrrolidin-1-yl)cyclohexanamine (480 mg, 36%) as white solid. ¹H NMR (400 MHz, DMSO-d₆): δ 7.42-7.14 (m, 10H), 3.56 (s, 4H), 2.88 (t, J=13.6 Hz, 2H), 2.69 (t, J=6.6 Hz, 2H), 2.37 (t, J=10.9 Hz, 1H), 2.23-2.10 (m, 2H), 2.10-2.02 (m, 1H), 1.90 (br d, J=11.5 Hz, 2H), 1.82 (d, J=11.5 Hz, 2H), 1.38 (q, J=11.5 Hz, 2H), 1.04-0.89 (m, 2H).

Step 2: A solution of N,N-dibenzyl-4-(3,3-difluoropyrrolidin-1-yl)cyclohexanamine (186 mg, 0.483 mmol) in MeOH (5 ml) was treated with Pd(OH) 2 (42 mg, 0.060 mmol, 20% purity). The mixture was degassed with H₂ (3×) and stirred at 50° C. for 12 h under H₂ atmosphere (50 psi). The mixture was filtered through Celite and the filtrate concentrated to afford (1r,4r)-4-(3,3-difluoropyrrolidin-1-yl)cyclohexan-1-amine (90 mg, 91%) as a colourless oil. ¹H NMR (DMSO-d₆, 400 MHz): δ 3.20-4.10 (m, 2H), 2.88 (t, J=13.8 Hz, 2H), 2.70 (t, J=6.9 Hz, 2H), 2.4-2.5 (m, 1H), 2.10-2.30 (m, 2H), 2.00 (tt, J=3.7, 10.7 Hz, 1H), 1.82 (d, J=12.1 Hz, 2H), 1.60-1.80 (m, 2H), 0.90-1.20 (m, 4H).

Step 3: A solution of 4-(3,3-difluoropyrrolidin-1-yl)cyclohexanamine (70 mg, 0.34 mmol), 2-(1H-imidazol-1-yl)pyrrolo[2,1-f][1,2,4]triazine-4-carboxylic acid (Intermediate 5) (78.6 mg, 0.343 mmol) and triethylamine (104 mg, 1.03 mmol) in CH₂Cl₂ (1 ml) was treated with T3P (327 mg, 0.514 mmol, 50% in EtOAc), stirred at 25° C. for 1 h, diluted with H₂O (10 ml) and extracted with CH₂Cl₂ (3×10 ml). The combined organic layers were washed with brine (30 mL), dried over Na₂SO₄, filtered and concentrated under reduced pressure. Purification by prep. HPLC (Welch Xtimate C18 150*30 mm*5 μm; mobile phase A: 0.05% NH₃ and 10 nM NH₄CO₃ in H₂O, mobile phase B: CH₃CN, 27 to 65% B) and lyophilization gave the title compound (26 mg, 18%) as yellow solid. ¹H NMR (DMSO-d₆, 400 MHz): δ 9.06-8.83 (m, 2H), 8.39-8.21 (m, 1H), 8.13-7.95 (m, 1H), 7.68-7.50 (m, 1H), 7.19 (s, 2H), 3.86 (s, 1H), 3.05-2.86 (m, 3H), 2.76 (s, 2H), 2.26-2.14 (m, 2H), 2.04-1.94 (m, 2H), 1.93-1.81 (m, 2H), 1.57 (d, J=3.4 Hz, 2H), 1.32-1.19 (m, 2H). MS (ES⁺): 416.1 [M+H]⁺.

### Example 13: N-((1S,4r)-4-((S)-3-fluoropyrrolidin-1-yl)cyclohexyl)-2-(1H-imidazol-1-yl)pyrrolo[2,1-f][1,2,4]triazine-4-carboxamide

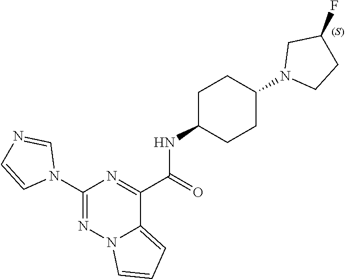

Prepared as described for Example 12 using 4-(dibenzylamino)cyclohexanone (1.2 g, 4.09 mmol) and (3S)-3-fluoropyrrolidine (514 mg, 4.09 mmol) to give the title compound (60 mg, 3.7%) as a yellow solid. ¹H NMR (400 MHz, DMSO-d₆): δ 9.02-8.92 (m, 2H), 8.31 (dd, J=1.4, 2.4 Hz, 1H), 8.06 (t, J=1.3 Hz, 1H), 7.59 (dd, J=1.3, 4.6 Hz, 1H), 7.23-7.16 (m, 2H), 5.36-5.08 (m, 1H), 3.95-3.79 (m, 1H), 3.04-2.64 (m, 3H), 2.46 (d, J=3.1 Hz, 1H), 2.20-1.97 (m, 4H), 1.96-1.82 (m, 3H), 1.66-1.51 (m, 2H), 1.37-1.25 (m, 2H). MS (ES⁺): 398.4 [M+H]⁺.

### Example 14: N-((1R,4r)-4-((R)-3-fluoropyrrolidin-1-yl)cyclohexyl)-2-(1H-imidazol-1-yl)pyrrolo[2,1-f][1,2,4]triazine-4-carboxamide

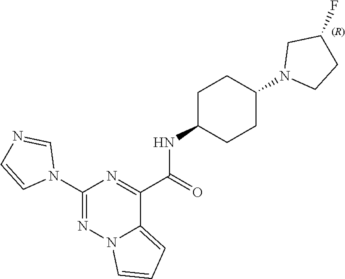

Prepared as described for Example 12 using 4-(dibenzylamino)cyclohexanone (1.00 g, 3.41 mmol) and (3R)-3-fluoropyrrolidine hydrochloride (428 mg, 3.41 mmol) to give the title compound (37 mg, 3%) as a yellow solid. ¹H NMR (400 MHz, DMSO-d₆): δ 8.97 (t, J=4.3 Hz, 2H), 8.31 (dd, J=1.3, 2.2 Hz, 1H), 8.06 (s, 1H), 7.60 (dd, J=1.1, 4.6 Hz, 1H), 7.24-7.15 (m, 2H), 5.30-5.07 (m, 1H), 3.96-3.79 (m, 1H), 2.96-2.80 (m, 2H), 2.76-2.60 (m, 1H), 2.39 (q, J=7.5 Hz, 1H), 2.13-1.97 (m, 4H), 1.93-1.81 (m, 3H), 1.66-1.52 (m, 2H), 1.31-1.24 (m, 2H). MS (ES⁺): 398.2 [M+H]⁺.

### Example 15:1-(1H-imidazol-1-yl)-N-((1r,4r)-4-((2,2,2-trifluoroethyl)amino)cyclohexyl)pyrrolo[1,2-a]pyrazine-3-carboxamide

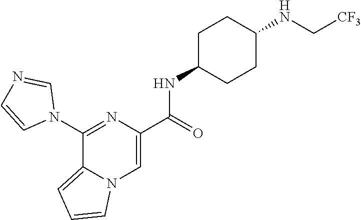 [SMILES: O=C(N[C@H]1CC[C@H](NCC(F)(F)F)CC1)c1cn2cccc2c(-n2ccnc2)n1]

A solution of 1-(1H-imidazol-1-yl)pyrrolo[1,2-a]pyrazine-3-carboxylic acid (Intermediate 6) (30 mg, 0.131 mmol) and (1r,4r)-N¹-(2,2,2-trifluoroethyl)cyclohexane-1,4-diamine hydrochloride (45.9 mg, 0.197 mmol) in CH₂Cl₂ (1 ml) was treated with T3P (125 mg, 0.197 mmol, 50% in EtOAc) and triethylamine (66 mg, 0.66 mmol) and stirred at 25° C. for 1 h. The mixture was diluted with H₂O (10 ml) and extracted with CH₂Cl₂ (3×10 ml). The combined organic layers were evaporated. The resulting residue was purified by prep. HPLC (Phenomenex C18 75*30 mm*3 μm, mobile phase A: 0.05% NH₃ and 10 nM NH₄CO₃ in H₂O, mobile phase B: CH₃CN, 25 to 45% B) and lyophilized to give the title compound (10 mg, 19%) as a white solid. ¹H NMR (400 MHz, DMSO-d₆): δ 9.01 (d, J=0.9 Hz, 1H), 8.85 (s, 1H), 8.27 (t, J=1.3 Hz, 1H), 8.23 (d, J=8.6 Hz, 1H), 8.15 (dd, J=1.2, 2.6 Hz, 1H), 7.33 (d, J=4.3 Hz, 1H), 7.22 (s, 1H), 7.11 (dd, J=2.6, 4.3 Hz, 1H), 3.88-3.75 (m, 1H), 3.29-3.19 (m, 2H), 2.44 (t, J=9.8 Hz, 1H), 2.23 (s, 1H), 1.94 (d, J=11.5 Hz, 2H), 1.82 (d, J=10.1 Hz, 2H), 1.58-1.46 (m, 2H), 1.18-1.06 (m, 2H). MS (ES⁺): 407.1 [M+H]⁺.

### Example 16: 4-(1H-imidazol-1-yl)-N-((1r,4r)-4-((2,2,2-trifluoroethyl)amino)cyclohexyl)pyrrolo[2,1-f][1,2,4]triazine-2-carboxamide

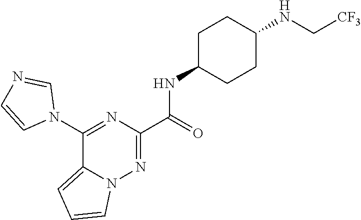 [SMILES: O=C(N[C@H]1CC[C@H](NCC(F)(F)F)CC1)c1nc(-n2ccnc2)c2cccn2n1]

A solution of 4-(1H-imidazol-1-yl)pyrrolo[2,1-f][1,2,4]triazine-2-carboxylic acid (Intermediate 7) (80 mg) and (1r,4r)-N¹-(2,2,2-trifluoroethyl)cyclohexane-1,4-diamine hydrochloride (82.2 mg, 0.353 mmol) in CH₂Cl₂ (1 ml) was treated with T3P (333 mg, 0.524 mmol, 50% in EtOAc) and DIPEA (135 mg, 1.05 mmol) and stirred at 25° C. for 1 h. The mixture was diluted with EtOAc (30 ml) and washed with H₂O (3×20 ml) and brine (20 ml). The combined organic layers were dried over Na₂SO₄, filtered and concentrated under reduced pressure. Purification of the residue by prep. HPLC (Xtimate C18 100*30 mm*10 μm, mobile phase A: 0.223% HCOOH in H₂O, mobile phase B: CH₃CN, 0 to 30% B) and lyophilization gave the title compound (8 mg, 5%) as a light yellow solid. ¹H NMR (400 MHz, DMSO-d₆): δ 9.08 (s, 1H), 8.64 (d, J=8.5 Hz, 1H), 8.41 (s, 2H), 7.63 (d, J=3.9 Hz, 1H), 7.35-7.21 (m, 2H), 3.86-3.76 (m, 1H), 3.26 (d, J=10.1 Hz, 2H), 2.44 (s, 1H), 1.95 (d, J=11.9 Hz, 1H), 1.83 (d, J=10.3 Hz, 2H), 1.51 (s, 2H), 1.20-1.11 (m, 2H), 0.97-0.89 (m, 2H). MS (ES⁺): 408.4 [M+H]⁺.

### Example 17: 2-(1-methyl-1H-imidazol-5-yl)-N-((1r,4r)-4-((2,2,2-trifluoroethyl)amino)cyclohexyl)pyrrolo[2,1-f][1,2,4]triazine-4-carboxamide

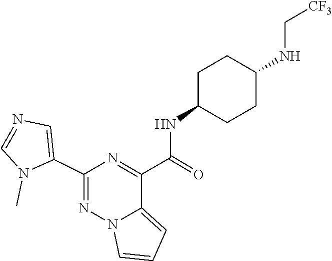 [SMILES: Cn1cncc1-c1nc(C(=O)N[C@H]2CC[C@H](NCC(F)(F)F)CC2)c2cccn2n1]

A solution of 2-(1-methyl-1H-imidazol-5-yl)pyrrolo[2,1-f][1,2,4]triazine-4-carboxylic acid (Intermediate 8) (110 mg, 0.452 mmol) and (1r,4r)-N¹-(2,2,2-trifluoroethyl)cyclohexane-1,4-diamine hydrochloride (105 mg, 0.452 mmol) in CH₂Cl₂ (3 ml) was treated with T3P (432 mg, 0.678 mmol, 50% in EtOAc) and triethylamine (229 mg, 2.26 mmol) and stirred at 25° C. for 1 h. The mixture was concentrated under reduced pressure to give a residue, which was purified by prep. HPLC (Phenomenex C18 75*30 mm*3 μm, mobile phase A: 0.05% NH₃ and 10 nM NH₄CO₃ in H₂O, mobile phase B: CH₃CN, 28 to 58% B) and lyophilized to give the title compound (11 mg, 6%) as a yellow solid. ¹H NMR (400 MHz, DMSO-d₆): δ 8.69 (d, J=8.6 Hz, 1H), 8.20 (dd, J=1.4, 2.4 Hz, 1H), 8.06 (d, J=1.0 Hz, 1H), 7.86 (s, 1H), 7.44 (dd, J=1.3, 4.6 Hz, 1H), 7.11 (dd, J=2.5, 4.5 Hz, 1H), 4.03 (s, 3H), 3.91-3.73 (m, 1H), 3.29-3.20 (m, 3H), 2.29-2.18 (m, 1H), 2.00-1.91 (m, 2H), 1.85 (dd, J=2.7, 12.1 Hz, 2H), 1.61-1.47 (m, 2H), 1.20-1.06 (m, 2H). MS (ES⁺): 422.2 [M+H]⁺.

### Example 18: 2-(1-(2-hydroxyethyl)-1H-imidazol-5-yl)-N-((1r,4r)-4-((2,2,2-trifluoroethyl)amino)cyclohexyl)pyrrolo[2,1-f][1,2,4]triazine-4-carboxamide

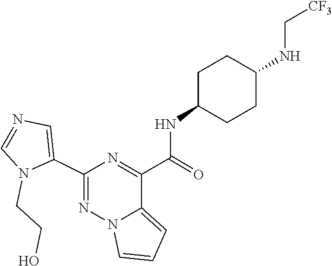 [SMILES: O=C(N[C@H]1CC[C@H](NCC(F)(F)F)CC1)c1nc(-c2cncn2CCO)nn2cccc12]

Step 1: A solution of 2-chloropyrrolo[2,1-f][1,2,4]triazine-4-carboxylic acid (Intermediate 12) (350 mg, 1.77 mmol), (1r,4r)-N¹-(2,2,2-trifluoroethyl)cyclohexane-1,4-diamine (417 mg, 2.13 mmol) and triethylamine (896 mg, 8.86 mmol) in CH₂Cl₂ (6 ml) was treated with T3P (1.69 g, 2.66 mmol, 50% in EtOAc) and stirred at 25° C. for 1 h. The mixture was concentrated under reduced pressure to give a residue, which was purified by flash chromatography (SepaFlash® Silica Flash Column, EtOAc in petroleum ether 1:1) to give 2-chloro-N-((1r,4r)-4-((2,2,2-trifluoroethyl)amino)cyclohexyl)pyrrolo[2,1-f][1,2,4]triazine-4-carboxamide (330 mg, 49%) as a yellow solid. ¹H NMR (400 MHz, DMSO-d₆): δ 8.76 (d, J=8.4 Hz, 1H), 8.34 (dd, J=1.3, 2.3 Hz, 1H), 7.50 (dd, J=1.1, 4.7 Hz, 1H), 7.22 (dd, J=2.5, 4.6 Hz, 1H), 3.86-3.70 (m, 1H), 3.30-3.18 (m, 2H), 2.46-2.35 (m, 1H), 2.21 (q, J=7.3 Hz, 1H), 1.93 (br d, J=11.6 Hz, 2H), 1.82 (d, J=10.5 Hz, 2H), 1.56-1.41 (m, 2H), 1.17-1.04 (m, 2H).

Step 2: A solution of imidazole (2.00 g, 29.4 mmol) and Cs₂CO₃ (19.1 g, 58.8 mmol) in CH₃CN (30 ml) was treated with tert-butyl-(2-iodoethoxy)-dimethyl-silane (10.1 g, 35.3 mmol) at 0° C., heated to 60° C. and stirred for 8 h. The mixture was cooled to RT, diluted with H₂O (20 ml) and extracted with EtOAc (3×30 ml). The combined organic layers were washed brine (30 ml), dried over anhydrous Na₂SO₄, filtered and concentrated under reduced pressure. Purification of the residue by flash chromatography (20 g SepaFlash® Silica Flash Column, EtOAc in petroleum ether 1:1) gave tert-butyl-(2-imidazol-1-ylethoxy)-dimethyl-silane (4.40 g, 63%) as a yellow oil. ¹H NMR (DMSO-d₆, 400 MHz): δ 7.56 (s, 1H), 7.1-7.2 (m, 1H), 6.80-6.90 (m, 1H), 4.00-4.10 (m, 2H), 3.79 (dd, J=3.0, 4.8 Hz, 2H), 0.80-0.82 (m, 9H), 0.10 (br. s, 6H).

Step 3: A solution of tert-butyl-(2-imidazol-1-ylethoxy)-dimethyl-silane (2.40 g, 10.6 mmol) in CHCl₃ (25 ml) was treated with NBS (1.13 g, 6.36 mmol) and stirred at 40° C. for 3 h. The mixture was concentrated under reduced pressure to dryness and the residue purified by flash chromatography (12 g SepaFlash® Silica Flash Column, 20% EtOAc in petroleum ether) to give 5-bromo-1-(2-((tert-butyldimethylsilyl)oxy)ethyl)-1H-imidazole (350 mg, 10%) as a colourless oil. ¹H NMR (DMSO-d₆, 400 MHz): δ 7.31 (d, J=1.4 Hz, 1H), 6.92 (d, J=1.3 Hz, 1H), 4.0-4.0 (m, 2H), 3.83 (t, J=5.2 Hz, 2H), 0.80 (s, 9H), —0.09 (s, 6H).

Step 4: A solution of 2-(5-bromoimidazol-1-yl) ethoxy-tert-butyl-dimethyl-silane (291 mg, 0.952 mmol) in THF (6 ml) was dropwise treated with n-BuLi (2.5 M in hexane, 0.762 ml) at −78° C., stirred for 20 min and dropwise treated with tributyl(chloro) stannane (1.24 g, 3.81 mmol). The mixture was stirred at −78° C. for 40 min and at 25° C. for 12 h before being quenched by the addition sat. aq. KF solution (10 ml) and sat. aq. NH₄Cl solution (10 ml). The mixture was extracted with EtOAc (3×20 ml). The combined organic layers were washed with brine (40 ml), dried over Na₂SO₄, filtered and concentrated under reduced pressure to dryness to give tert-butyl-dimethyl-[2-(5-tributylstannylimidazol-1-yl) ethoxy]silane (400 mg) as a light yellow solid (used in the next step without further purification).

Step 5: A mixture of tert-butyl-dimethyl-[2-(5-tributylstannylimidazol-1-yl) ethoxy]silane (400 mg, 0.776 mmol), 2-chloro-N-[4-(2,2,2-trifluoroethylamino)cyclohexyl]pyrrolo[2,1-f][1,2,4]triazine-4-carboxamide (330 mg, 0.877 mmol) and Pd(PPh₃) 4 (179 mg, 0.155 mmol) in dioxane (5 ml) was degassed with N₂ (3×) and stirred at 100° C. for 12 h under N₂ atmosphere. The mixture was filtered and concentrated under reduced pressure to give a residue, which was purified by flash chromatography (4 g SepaFlash® Silica Flash Column, EtOAc/petrol ether 1:1) and further purified by prep. HPLC (Welch Ultimate XB-CN 250*50*10 μm; mobile phase A: 0.05% NH₃ and 10 nM NH₄CO₃ in H₂O, mobile phase B: CH₃CN, 5 to 29% B). Lyophilization gave 2-(1-(2-((tert-butyldimethylsilyl)oxy)ethyl)-1H-imidazol-5-yl)-N-((1r,4r)-4-((2,2,2-trifluoroethyl)amino)cyclohexyl)pyrrolo[2,1-f][1,2,4]triazine-4-carboxamide (10 mg, 2%) as light yellow solid. ¹H NMR (400 MHz, MeOD-d₄): δ 8.05-7.98 (m, 2H), 7.82 (s, 1H), 7.52 (dd, J=1.2, 4.6 Hz, 1H), 7.07 (dd, J=2.6, 4.6 Hz, 1H), 4.72 (t, J=4.8 Hz, 2H), 4.00 (t, J=4.8 Hz, 2H), 3.97-3.87 (m, 1H), 3.29-3.21 (m, 2H), 2.67-2.55 (m, 1H), 2.10-2.03 (m, 4H), 1.63-1.52 (m, 2H), 1.33-1.25 (m, 2H), 0.79 (s, 9H), −0.13 (s, 6H).

Step 6: A solution of 2-(1-(2-((tert-butyldimethylsilyl)oxy)ethyl)-1H-imidazol-5-yl)-N-((1r,4r)-4-((2,2,2-trifluoroethyl)amino)cyclohexyl)pyrrolo[2,1-f][1,2,4]triazine-4-carboxamide (7 mg, 0.012 mmol) in CH₂Cl₂ (0.5 mL) was treated with 4 M HCl in dioxane (4 M, 0.062 mL) and stirred at 25° C. for 30 min. The mixture was concentrated under reduced pressure to give a residue, which was purified by prep. HPLC (Phenomenex C18 75*30 mm*3 μm; mobile phase A: 0.05% NH₃ and 10 nM NH₄CO₃ in H₂O, mobile phase B: CH₃CN, 14 to 44% B) and lyophilized to give the title compound (3 mg, 59%) as a yellow solid. ¹H NMR (400 MHz, DMSO-d₆): δ 8.68 (d, J=8.6 Hz, 1H), 8.18 (dd, J=1.4, 2.3 Hz, 1H), 8.05 (d, J=0.8 Hz, 1H), 7.84 (s, 1H), 7.43 (dd, J=1.3, 4.5 Hz, 1H), 7.11 (dd, J=2.6, 4.6 Hz, 1H), 4.95 (s, 1H), 4.54 (t, J=5.5 Hz, 2H), 3.88-3.78 (m, 1H), 3.74 (s, 2H), 3.28-3.21 (m, 2H), 2.47-2.40 (m, 1H), 2.28-2.20 (m, 1H), 1.95 (d, J=11.6 Hz, 2H), 1.85 (d, J=10.6 Hz, 2H), 1.59-1.48 (m, 2H), 1.19-1.08 (m, 2H). MS (ES⁺): 452.2 [M+H]⁺.

### Example 19: 8-(1H-imidazol-1-yl)-N-((1r,4r)-4-((2,2,2-trifluoroethyl)amino)cyclohexyl) imidazo[1,2-a]pyrazine-6-carboxamide

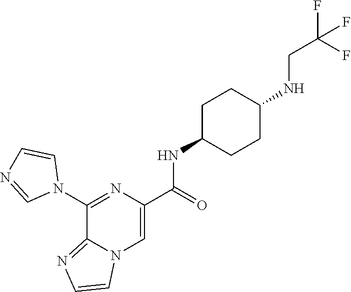 [SMILES: O=C(N[C@H]1CC[C@H](NCC(F)(F)F)CC1)c1cn2ccnc2c(-n2ccnc2)n1]

A solution of 8-(1H-imidazol-1-yl) imidazo[1,2-a]pyrazine-6-carboxylic acid (Intermediate 9) (110 mg, 0.480 mmol) and N⁴-(2,2,2-trifluoroethyl)cyclohexane-1,4-diamine (94.2 mg, 0.480 mmol) in CH₂Cl₂ (3 ml) was treated with T3P (458 mg, 0.720 mmol, 50% in EtOAc) and triethylamine (242.8 mg, 2.40 mmol) and stirred at 25° C. for 1 h. The mixture was concentrated under reduced pressure to give a residue, which was purified by prep. HPLC (Phenomenex C18 75*30 mm*3 μm, mobile phase A: 0.05% NH₃ and 10 nM NH₄CO₃ in H₂O, mobile phase B: CH₃CN, 25 to 46% B 25% B) and further purified by prep. HPLC (Phenomenex 150*30 mm*5 μm, mobile phase A: H₂O, mobile phase B: CH₃CN, 0 to 25% B). Lyophilization of the pure fractions gave the title compound (8 mg, 79%) as a white solid. ¹H NMR (400 MHz, DMSO-d₆): δ 10.85 (s, 1H), 10.35-10.10 (m, 1H), 9.45 (s, 1H), 9.34 (s, 1H), 8.94 (d, J=8.8 Hz, 1H), 8.55 (s, 1H), 8.09 (s, 1H), 8.02 (s, 1H), 4.12 (q, J=9.6 Hz, 2H), 3.99-3.83 (m, 1H), 3.17 (s, 1H), 2.27 (s, 2H), 1.91 (s, 2H), 1.80-1.59 (m, 4H). MS (ES⁺): 408.1 [M+H]⁺.

### Example 20: 8-(1H-imidazol-1-yl)-N-((1r,4r)-4-((2,2,2-trifluoroethyl)amino)cyclohexyl)-[1,2,4]triazolo[1,5-a]pyrazine-6-carboxamide

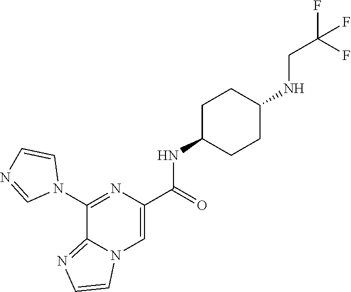 [SMILES: O=C(N[C@H]1CC[C@H](NCC(F)(F)F)CC1)c1cn2ccnc2c(-n2ccnc2)n1]

Step 1: A solution of 6,8-dibromo-[1,2,4]triazolo[1,5-a]pyrazine (1.00 g, 3.60 mmol) and imidazole (245 mg, 3.60 mmol) in DMF (10 ml) was treated with DIPEA (1.40 g, 10.80 mmol) and stirred at 100° C. for 12 h. The mixture was diluted with H₂O (40 ml) and extracted with EtOAc (3×40 ml). The combined organic layers were washed with brine (30 ml), dried over Na₂SO₄, filtered and concentrated under reduced pressure to give a residue, which was purified by flash chromatography (12 g SepaFlash®) Silica Flash, EtOAc in petroleum ether 0 to 50%) to give 6-bromo-8-imidazol-1-yl-[1,2,4]triazolo[1,5-a]pyrazine (590 mg, 62%) as a red brown solid. ¹H NMR (400 MHz, DMSO-d₆): δ 9.51 (s, 1H), 9.06 (s, 1H), 8.88 (s, 1H), 8.29 (t, J=1.3 Hz, 1H), 7.27 (s, 1H).

Step 2: A mixture of 6-bromo-8-imidazol-1-yl-[1,2,4]triazolo[1,5-a]pyrazine (300 mg, 1.13 mmol), N⁴-(2,2,2-trifluoroethyl)cyclohexane-1,4-diamine (511 mg, 2.60 mmol), AcONa (4641 mg, 5.66 mmol), Pd(dppf)Cl₂·CH₂Cl₂ (277 mg, 0.340 mmol) in DMF (20 ml) was degassed with CO (3×) and stirred at 80° C. for 48 h under CO atmosphere (50 psi). The mixture was filtered and concentrated under reduced pressure to give a residue, which was purified by prep. HPLC (Xtimate C18 100*30 mm*10 μm, mobile phase A: 0.223% HCOOH in H₂O, mobile phase B: CH₃CN, 0 to 30% B). Lyophilization of the pure fractions gave the title compound (25 mg, 25%) as a white solid. ¹H NMR (400 MHz, DMSO-d₆): δ 9.39 (s, 1H), 9.33 (s, 1H), 8.97 (s, 1H), 8.79 (t, J=1.3 Hz, 1H), 8.63 (d, J=8.6 Hz, 1H), 7.28 (d, J=0.9 Hz, 1H), 3.95-3.80 (m, 1H), 3.27 (d, J=10.1 Hz, 3H), 2.46-2.41 (m, 1H), 2.03-1.92 (m, 2H), 1.83 (d, J=10.8 Hz, 2H), 1.65-1.51 (m, 2H), 1.20-1.08 (m, 2H). MS (ES⁺): 409.0 [M+H]⁺.

### Example 21: 4-(1H-imidazol-1-yl)-N-((1r,4r)-4-((2,2,2-trifluoroethyl)amino)cyclohexyl)pyrazolo[1,5-a]pyrazine-6-carboxamide

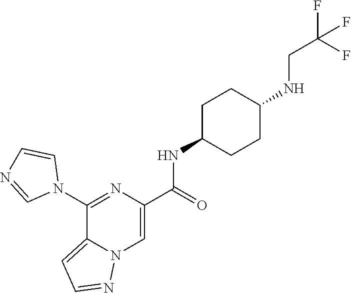 [SMILES: O=C(N[C@H]1CC[C@H](NCC(F)(F)F)CC1)c1cn2nccc2c(-n2ccnc2)n1]

Step 1: A solution of 4,6-dichloropyrazolo[1,5-a]pyrazine (500 mg, 2.66 mmol) and imidazole (181 mg, 2.66 mmol) in DMF (6 ml) was treated with DIPEA (1.03 g, 7.98 mmol) and stirred at 100° C. for 4 h. The mixture was concentrated under reduced pressure and the residue purified by flash chromatography (12 g SepaFlash® Silica Flash, EtOAc in petroleum ether 0 to 70%) to give 6-chloro-4-imidazol-1-yl-pyrazolo[1,5-a]pyrazine (500 mg, 86%) as a yellow solid. ¹H NMR (400 MHz, DMSO-d₆): δ 9.20 (s, 1H), 8.64 (s, 1H), 8.35 (d, J=2.5 Hz, 1H), 8.04 (t, J=1.3 Hz, 1H), 7.54-7.50 (m, 1H), 7.25 (s, 1H).

Step 2: A mixture of 6-chloro-4-imidazol-1-yl-pyrazolo[1,5-a]pyrazine (50 mg, 0.228 mmol), N⁴-(2,2,2-trifluoroethyl)cyclohexane-1,4-diamine hydrochloride (106 mg, 0.455 mmol), triethylamine (230 mg, 2.28 mmol), 1,3-bis(diphenylphosphino) propane (DPPP, 46.9 mg, 0.114 mmol) and Pd(OAc)₂ (10.2 mg, 0.0455 mmol) in DMF (2 ml) was degassed with CO (3×) and stirred at 80° C. for 16 h under CO atmosphere. The mixture was filtered and concentrated under reduced pressure to give a residue, which was purified by prep. HPLC (Phenomenex C18 75*30 mm*3 μm, mobile phase A: 0.05% NH₃ and 10 nM NH₄CO₃ in H₂O, mobile phase B: CH₃CN, 18 to 48% B) and further purified by prep. HPLC (Welch Xtimate C18 150*25 mm*5 μm, mobile Phase A: 0.225% HCOOH in H₂O, mobile phase B: CH₃CN, 0 to 30%). Lyophilization of the pure fractions gave the title compound (10 mg, 11%) as a white solid. ¹H NMR (400 MHz, DMSO-d₆): δ 9.10 (d, J=0.9 Hz, 1H), 8.99 (s, 1H), 8.48-8.43 (m, 2H), 8.41 (s, 1H), 7.59 (dd, J=0.9, 2.4 Hz, 1H), 7.27 (s, 1H), 3.92-3.79 (m, 1H), 3.28-3.22 (m, 2H), 3.16 (q, J=7.0 Hz, 1H), 2.46-2.40 (m, 1H), 1.96 (d, J=11.6 Hz, 2H), 1.86-1.79 (m, 2H), 1.63-1.49 (m, 2H), 1.19-1.09 (m, 2H). MS (ES⁺): 408.2 [M+H]⁺.

### Example 22: N-((1S,4r)-4-((S)-3-fluoropyrrolidin-1-yl)cyclohexyl)-1-(1H-imidazol-1-yl)pyrrolo[1,2-a]pyrazine-3-carboxamide

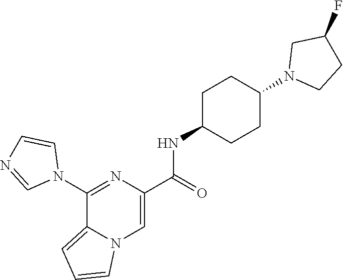 [SMILES: O=C(N[C@H]1CC[C@H](N2CC[C@H](F)C2)CC1)c1cn2cccc2c(-n2ccnc2)n1]

A mixture of 1-(1H-imidazol-1-yl)pyrrolo[1,2-a]pyrazine-3-carboxylic acid (Intermediate 6) (15 mg, 0.066 mmol), 4-[(3S)-3-fluoropyrrolidin-1-yl]cyclohexanamine (12 mg, 0.066 mmol), triethylamine (19.9 mg, 0.197 mmol) in DMF (0.5 ml) was treated with HATU (30 mg, 0.079 mmol) and stirred at 25° C. for 2 h. The mixture was concentrated and the residue purified by prep. HPLC (Phenomenex C18 75×30 mm×3 μm, mobile phase A: 0.05% NH₃ and 10 nM NH₄CO₃ in H₂O, mobile phase B: CH₃CN, 10 to 50% B). Lyophilization of the pure fractions gave the title compound (5 mg, 15%) as a white solid. ¹H NMR (400 MHz, DMSO-d₆): δ 9.01 (s, 1H), 8.85 (s, 1H), 8.33-8.22 (m, 2H), 8.15 (d, J=1.5 Hz, 1H), 7.32 (d, J=4.3 Hz, 1H), 7.22 (s, 1H), 7.11 (dd, J=2.7, 4.1 Hz, 1H), 5.28-5.07 (m, 1H), 3.89-3.75 (m, 1H), 2.97-2.79 (m, 2H), 2.71 (br dd, J=3.3, 10.5 Hz, 1H), 2.67-2.57 (m, 1H), 2.38 (br d, J=6.3 Hz, 1H), 2.15-2.02 (m, 2H), 1.98 (br d, J=12.6 Hz, 2H), 1.84 (br d, J=10.1 Hz, 2H), 1.61-1.46 (m, 2H), 1.35-1.19 (m, 2H). MS (ES⁺): 397.0 [M+H]⁺.

### Example 23: N-((1S,4r)-4-((S)-3-hydroxypyrrolidin-1-yl)cyclohexyl)-2-(1H-imidazol-1-yl)pyrrolo[2,1-f][1,2,4]triazine-4-carboxamide

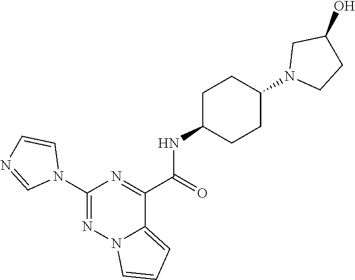 [SMILES: O=C(N[C@H]1CC[C@H](N2CC[C@H](O)C2)CC1)c1nc(-n2ccnc2)nn2cccc12]

Step 1: A mixture of 4-(dibenzylamino)cyclohexanone (300 mg, 1.02 mmol), NaBH(OAc)₃ (650 mg, 3.07 mmol), AcOH (184 mg, 3.07 mmol) in CH₂Cl₂ (5 ml) was treated with N,N-diisopropylethylamine (1 ml) and (3S)-pyrrolidin-3-ol hydrochloride (126 mg, 1.02 mmol), and stirred at 25° C. for 12 h. The mixture was treated with sat. aq. NaHCO₃ to reach pH>7 and extracted with CH₂Cl₂ (3×5 ml). The combined organic layers were washed with H₂O (3×5 ml), dried over Na₂SO₄, filtered and concentrated under reduced pressure to give a residue, which was purified by column chromatography (SiO₂, EtOAc in petroleum ether 0 to 10%) and further purified by prep. HPLC (Phenomenex Luna C18 100×30 mm×5 μm, mobile phase A: 0.223% HCOOH in H₂O, mobile phase B: CH₃CN, 0 to 30% B). Lyophilization of the pure fractions gave (3S)-1-[4-(dibenzylamino)cyclohexyl]pyrrolidin-3-ol (100 mg, 26%) as a white solid. ¹H NMR (400 MHz, DMSO-d₆): δ 7.36-7.15 (m, 10H), 4.11 (tt, J=3.5, 6.7 Hz, 1H), 3.56 (s, 4H), 2.78-2.69 (m, 1H), 2.65-2.56 (m, 1H), 2.46 (br. d, J=5.9 Hz, 1H), 2.41-2.29 (m, 2H), 2.03-1.89 (m, 4H), 1.81 (br. d, J=11.5 Hz, 2H), 1.57-1.44 (m, 1H), 1.44-1.30 (m, 2H), 1.06-0.86 (m, 2H).

Step 2: A mixture of (3S)-1-[4-(dibenzylamino)cyclohexyl]pyrrolidin-3-ol (100 mg, 0.274 mmol) in EtOH (2 ml) was treated with Pd(OH) 2 (38.5 mg, 0.549 mmol, 20%) and stirred at 25° C. for 12 h under H₂ (40 psi) atmosphere. The mixture was filtered and filtrate was concentrated to dryness to give (3S)-1-(4-aminocyclohexyl)pyrrolidin-3-ol (50 mg, 98%) as a white solid, which was used for next step without further purification. Step 3: A solution of (3S)-1-(4-aminocyclohexyl)pyrrolidin-3-ol (200 mg, 1.09 mmol) and 2-(1H-imidazol-1-yl)pyrrolo[2,1-f][1,2,4]triazine-4-carboxylic acid (Intermediate 5) (249 mg, 1.09 mmol) in DMF (2 ml) was treated with HATU (495 mg, 1.30 mmol) and triethylamine (549 mg, 5.43 mmol) and stirred at 25° C. for 12 h. The mixture was concentrated to dryness and the residue diluted with H₂O (10 ml) and extracted with EtOAc (3×10 ml). The combined organic layers were washed with H₂O (3×10 ml), dried over Na₂SO₄, filtered and concentrated under reduced pressure to give a residue, which was purified by prep. HPLC (Xtimate C18 100*30 mm*10 μm, mobile phase A: 0.223% HCOOH in H₂O, mobile phase B: CH₃CN, 0 to 30% B) and further purified by SFC (DAICEL CHIRALPAK AD (250 mm*30 mm, 10 μm); mobile phase: A: supercritical CO₂, B: 0.1% NH₃. H₂O in EtOH, A:B=55:45). Lyophilisation of the pure fractions gave the title compound (25 mg, 6%) as a yellow solid. ¹H NMR (400 MHz, MeOD-d₄): δ 8.92 (s, 1H), 8.19-8.16 (m, 1H), 8.14-8.06 (m, 1H), 7.74-7.65 (m, 1H), 7.21-7.18 (m, 2H), 4.43-4.34 (m, 1H), 4.03-3.92 (m, 1H), 3.05-2.98 (m, 1H), 2.88-2.80 (m, 1H), 2.78-2.71 (m, 1H), 2.63-2.57 (m, 1H), 2.27-2.21 (m, 1H), 2.19-2.12 (m, 3H), 2.11-2.05 (m, 2H), 1.81-1.72 (m, 1H), 1.69-1.58 (m, 2H), 1.51-1.42 (m, 2H). MS (ES⁺): 396.2 [M+H]⁺.

### Example 24: N-((1R,4r)-4-((R)-3-fluoropyrrolidin-1-yl)cyclohexyl)-1-(1H-imidazol-1-yl)pyrrolo[1,2-a]pyrazine-3-carboxamide

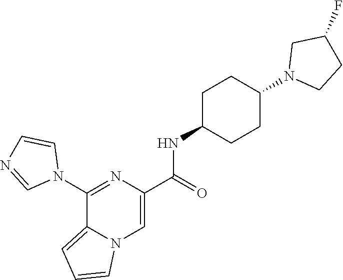 [SMILES: O=C(N[C@H]1CC[C@H](N2CC[C@@H](F)C2)CC1)c1cn2cccc2c(-n2ccnc2)n1]

A mixture of 1-(1H-imidazol-1-yl)pyrrolo[1,2-a]pyrazine-3-carboxylic acid (Intermediate 6) (8 mg, 0.035 mmol), 4-[(3R)-3-fluoropyrrolidin-1-yl]cyclohexanamine (6.5 mg, 0.035 mmol), HATU (16 mg, 0.042 mmol) in DMF (0.5 ml) was treated with triethylamine (10.6 mg, 0.105 mmol) and stirred at 25° C. for 2 h. The mixture was concentrated and purified by prep. HPLC (Phenomenex C18 75×30 mm×3 μm, mobile phase A: 0.05% NH₃ and 10 nM NH₄CO₃ in H₂O, mobile phase B: CH₃CN, 10 to 50% B). Lyophilization of the pure fractions gave the title compound (8.21 mg, 60%) as a white solid. ¹H NMR (400 MHz, DMSO-d₆): δ 9.01 (s, 1H), 8.85 (s, 1H), 8.30-8.20 (m, 2H), 8.19-8.10 (m, 1H), 7.32 (d, J=4.1 Hz, 1H), 7.28-7.18 (m, 1H), 7.11 (dd, J=2.6, 4.1 Hz, 1H), 5.31-5.02 (m, 1H), 3.96-3.73 (m, 1H), 2.93-2.79 (m, 2H), 2.74-2.65 (m, 1H), 2.65-2.57 (m, 1H), 2.41-2.31 (m, 1H), 2.20-2.02 (m, 2H), 1.98 (br. d, J=13.6 Hz, 2H), 1.87-1.81 (m, 2H), 1.60-1.47 (m, 2H), 1.32-1.20 (m, 2H). MS (ES⁺): 397.2 [M+H]⁺.

### Example 25: N-((1S,4r)-4-((S)-3-fluoropyrrolidin-1-yl)cyclohexyl)-8-(1H-imidazol-1-yl)-[1,2,4]triazolo[1,5-a]pyrazine-6-carboxamide

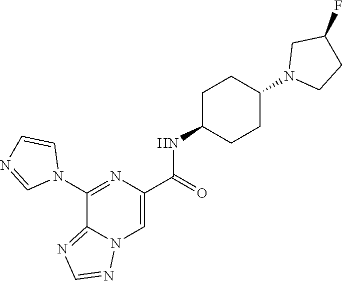 [SMILES: O=C(N[C@H]1CC[C@H](N2CC[C@H](F)C2)CC1)c1cn2ncnc2c(-n2ccnc2)n1]

Prepared as described for Example 26 using 6-bromo-8-imidazol-1-yl-[1,2,4]triazolo[1,5-a]pyrazine (50 mg, 0.189 mmol) and (1S,4r)-4-((S)-3-fluoropyrrolidin-1-yl)cyclohexan-1-amine (35 mg, 0.189 mmol) to give the title compound (3 mg, 4%) as a white solid. ¹H NMR (400 MHz, DMSO-d₆): δ 9.40 (s, 1H), 9.33 (s, 1H), 8.97 (s, 1H), 8.80 (s, 1H), 8.65 (d, J=8.63 Hz, 1H), 7.29 (s, 1H), 5.08-5.29 (m, 1H), 3.84-3.94 (m, 1H), 2.84 (s, 2H), 2.63-2.75 (m, 1H), 2.36-2.44 (m, 1H), 1.97-2.18 (m, 4H), 1.78-1.94 (m, 3H), 1.54-1.68 (m, 2H), 1.23-1.33 (m, 2H). MS (ES⁺): 399.1 [M+H]⁺.

### Example 26: N-((1R,4r)-4-((R)-3-fluoropyrrolidin-1-yl)cyclohexyl)-8-(1H-imidazol-1-yl)-[1,2,4]triazolo[1,5-a]pyrazine-6-carboxamide

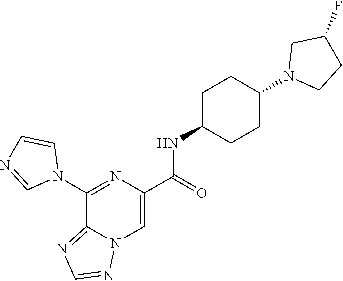 [SMILES: O=C(N[C@H]1CC[C@H](N2CC[C@@H](F)C2)CC1)c1cn2ncnc2c(-n2ccnc2)n1]

Step 1: A solution of 6,8-dibromo-[1,2,4]triazolo[1,5-a]pyrazine (800 mg, 2.88 mmol), N,N-diisopropylethylamine (1.12 g, 8.64 mmol) and imidazole (196 mg, 2.88 mmol) in DMF (8 ml) was stirred at 100° C. for 1 h and concentrated under reduced pressure. The residue was diluted with H₂O (10 ml) and extracted with EtOAc (3×10 ml). The combined organic layers were washed with brine (10 ml), dried over Na₂SO₄, filtered and concentrated under reduced pressure to give a residue, which was purified by flash chromatography (20 g SepaFlash® Silica Flash, EtOAc in petroleum ether 0 to 50%) to give 6-bromo-8-imidazol-1-yl-[1,2,4]triazolo[1,5-a]pyrazine (850 mg, 99%) as a yellow solid. ¹H NMR (400 MHz, DMSO-d₆): δ 9.51 (s, 1H), 9.06 (t, J=1.0 Hz, 1H), 8.88 (s, 1H), 8.29 (t, J=1.4 Hz, 1H), 7.26 (dd, J=0.9, 1.5 Hz, 1H).

Step 2: A mixture of 6-bromo-8-imidazol-1-yl-[1,2,4]triazolo[1,5-a]pyrazine (150 mg, 0.566 mmol), (1R,4r)-4-((R)-3-fluoropyrrolidin-1-yl)cyclohexan-1-amine (105 mg, 0.566 mmol), Pd(dppf)Cl₂·CH₂Cl₂ (139 mg, 0.169 mmol) and sodium acetate (232 mg, 2.83 mmol) in DMF (0.5 ml) was stirred at 80° C. for 100 h under CO atmosphere (50 psi). The mixture was filtered and concentrated under reduced pressure. The resulting residue was purified by prep. HPLC (Phenomenex C18 75*30 mm*3 μm, mobile phase A: 0.05% NH₃ and 10 nM NH₄CO₃ in H₂O, mobile phase B: CH₃CN, 6 to 46% B) and lyophilized to give the title compound (28 mg, 12%) as a white solid. ¹H NMR (400 MHz, DMSO-d₆): δ 9.38-9.43 (m, 1H), 9.33 (s, 1H), 8.97 (s, 1H), 8.80 (s, 1H), 8.65 (d, J=8.76 Hz, 1H), 7.22-7.39 (m, 1H), 5.08-5.29 (m, 1H), 3.79-4.01 (m, 1H), 2.79-2.96 (m, 2H), 2.63-2.75 (m, 1H), 2.35-2.43 (m, 1H), 1.99-2.16 (m, 4H), 1.86 (br. d, J=14.5 Hz, 3H), 1.56-1.67 (m, 2H), 1.23-1.34 (m, 2H). MS (ES⁺): 399.2 [M+H]⁺.

### Example 27: N-((1R,4r)-4-((R)-3-hydroxypyrrolidin-1-yl)cyclohexyl)-2-(1H-imidazol-1-yl)pyrrolo[2,1-f][1,2,4]triazine-4-carboxamide

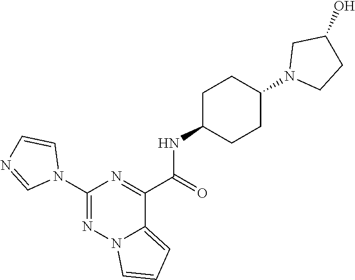 [SMILES: O=C(N[C@H]1CC[C@H](N2CC[C@@H](O)C2)CC1)c1nc(-n2ccnc2)nn2cccc12]

Step 1: A mixture of (3R)-pyrrolidin-3-ol hydrochloride (842 mg, 6.82 mmol) in CH₂Cl₂ (30 ml) and MeOH (5 ml) was treated with 4-(dibenzylamino)cyclohexanone (2.00 g, 6.82 mmol), NaBH(OAc)₃ (4.33 g, 20.5 mmol) and AcOH (1.23 g, 20.45 mmol) and stirred at 25° C. for 12 h. The mixture was treated with aq. sat. NaHCO₃ to adjust the pH>7 and extracted with CH₂Cl₂ (3×50 ml). The combined organic layers were washed with H₂O (3×50 ml), dried over Na₂SO₄, filtered and concentrated under reduced pressure. The resulting residue was purified by flash chromatography (40 g SepaFlash®) Silica Flash, MeOH in CH₂Cl₂ 0 to 10%) to give (3R)-1-[4-(dibenzylamino)cyclohexyl]pyrrolidin-3-ol (900 mg, 36%) as a white solid. ¹H NMR (400 MHz, DMSO-d₆): δ 7.36-7.14 (m, 10H), 4.60 (d, J=4.4 Hz, 1H), 4.16-4.07 (m, 1H), 3.56 (s, 4H), 2.69 (dd, J=6.4, 9.5 Hz, 1H), 2.59-2.53 (m, 1H), 2.46-2.26 (m, 3H), 1.97-1.75 (m, 6H), 1.52-1.31 (m, 3H), 0.95 (q, J=12.3 Hz, 2H).

Step 2: A mixture of (3R)-1-[4-(dibenzylamino)cyclohexyl]pyrrolidin-3-ol (200 mg, 0.549 mmol) in EtOH (2 ml) was treated with Pd(OH) 2 (77.1 mg, 0.110 mmol, 20%) and stirred at 25° C. for 12 h under H₂ atmosphere (40 psi). The mixture was filtered and concentrated under reduced pressure to give (3R)-1-(4-aminocyclohexyl)pyrrolidin-3-ol (100 mg, 99%) as a white solid, which was used for next step without further purification.

Step 3: A solution of (3R)-1-(4-aminocyclohexyl)pyrrolidin-3-ol (100 mg, 0.543 mmol) and 2-(1H-imidazol-1-yl)pyrrolo[2,1-f][1,2,4]triazine-4-carboxylic acid (Intermediate 5) (124 mg, 0.543 mmol) in DMF (1.5 ml) was treated with HATU (247 mg, 0.651 mmol) and triethylamine (275 mg, 2.71 mmol) and stirred at 25° C. for 12 h. The mixture was concentrated to dryness and purified by prep. HPLC (Xtimate C18 100×30 mm×10 μm, mobile phase A: 0.223% HCOOH in H₂O, mobile phase B: CH₃CN, 5 to 25% B) and further purified by prep. HPLC (Phenomenex C18 75×30 mm×3 μm, mobile phase A: 0.05% NH₃ and 10 nM NH₄CO₃ in H₂O, mobile phase B: CH₃CN, 10 to 50% B). Lyophilization of the pure fractions gave the title compound (5 mg, 2%) as a yellow solid. ¹H NMR (400 MHz, DMSO-d₆): δ 8.98-8.95 (m, 1H), 8.31 (dd, J=1.4, 2.4 Hz, 1H), 8.17 (s, 1H), 8.06 (t, J=1.3 Hz, 1H), 7.59 (dd, J=1.3, 4.7 Hz, 1H), 7.22-7.15 (m, 2H), 4.95-4.69 (m, 1H), 4.21 (br. s, 1H), 3.86 (dtd, J=4.3, 7.8, 11.8 Hz, 2H), 2.94-2.77 (m, 3H), 2.29-2.14 (m, 1H), 2.06-1.94 (m, 3H), 1.88 (br d, J=11.5 Hz, 2H), 1.65-1.54 (m, 3H), 1.38-1.26 (m, 2H).

### Example 28: N-((1R,4r)-4-((R)-3-fluoropyrrolidin-1-yl)cyclohexyl)-4-(1H-imidazol-1-yl)pyrrolo[2,1-f][1,2,4]triazine-2-carboxamide

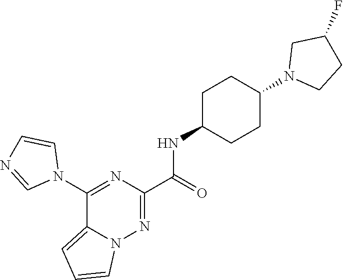 [SMILES: O=C(N[C@H]1CC[C@H](N2CC[C@@H](F)C2)CC1)c1nc(-n2ccnc2)c2cccn2n1]

A mixture of 4-(1H-imidazol-1-yl)pyrrolo[2,1-f][1,2,4]triazine-2-carboxylic acid (Intermediate 7) (25 mg, 0.109 mmol) and (1R,4r)-4-((R)-3-fluoropyrrolidin-1-yl)cyclohexan-1-amine (20.3 mg, 0.109 mmol) in CH₂Cl₂ (0.2 ml) was treated with N,N-diisopropylethylamine (42.3 mg, 0.327 mmol) and HATU (83.0 mg, 0.218 mmol) in one portion at 25° C. and stirred at 25° C. for 6 h. The mixture was poured into H₂O (10 ml) and extracted with CH₂Cl₂ (3×10 ml). The combined organic layers were concentrated and the residue purified by prep. HPLC (Welch Xtimate C18 150×40 mm×10 μm, mobile phase A: 0.05% NH₃ and 10 nM NH₄CO₃ in H₂O, mobile phase B: CH₃CN, 6 to 46% B). Lyophilization of the pure fractions gave the title compound (3 mg, 6%) as an off-white solid. ¹H NMR (400 MHz, DMSO-d₆): δ 9.09 (s, 1H), 8.68 (d, J=8.5 Hz, 1H), 8.51-8.30 (m, 2H), 7.64 (dd, J=1.1, 4.8 Hz, 1H), 7.33-7.25 (m, 2H), 5.35-5.08 (m, 1H), 3.93-3.73 (m, 1H), 3.06-2.73 (m, 3H), 2.10-1.98 (m, 3H), 1.92-1.74 (m, 3H), 1.62-1.49 (m, 2H), 1.34-1.22 (m, 4H). MS (ES⁺): 398.2 [M+H]⁺.

### Example 29: 2-(1H-imidazol-1-yl)-N-((1r,4r)-4-((2,2,2-trifluoroethyl)amino)cyclohexyl)pyrrolo[2,1-f][1,2,4]triazine-4-carboxamide

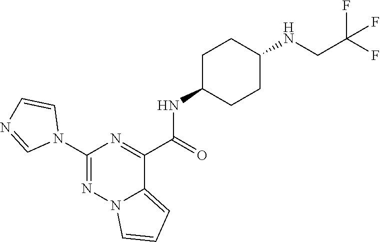 [SMILES: O=C(N[C@H]1CC[C@H](NCC(F)(F)F)CC1)c1nc(-n2ccnc2)nn2cccc12]

A solution of 2-(1H-imidazol-1-yl)pyrrolo[2,1-f][1,2,4]triazine-4-carboxylic acid (Intermediate 5) (80 mg, 0.349 mmol) and (1r,4r)-N¹-(2,2,2-trifluoroethyl)cyclohexane-1,4-diamine hydrochloride (122 mg, 0.524 mmol) in CH₂Cl₂ (2 mL) was treated with HATU (199 mg, 0.524 mmol) and triethylamine (106 mg, 1.05 mmol) and stirred at 25° C. for 2 h. The mixture was diluted with H₂O (10 ml) and extracted with CH₂Cl₂ (3×10 ml). The combined organic layers were washed with brine (20 ml), dried over Na₂SO₄, filtered and concentrated under reduced pressure to give a residue, which was purified by prep. HPLC (Phenomenex C18 75*30 mm*3 μm, mobile phase A: 0.05% NH₃ and 10 nM NH₄CO₃ in H₂O, mobile phase B: CH₃CN, 28 to 58% B). Lyophilization of the pure fractions gave the title compound (71 mg, 0.172 mmol, 49%) as a yellow solid. ¹H NMR (400 MHz, DMSO-d₆): δ 9.04-8.87 (m, 2H), 8.29 (s, 1H), 8.05 (s, 1H), 7.59 (d, J=3.8 Hz, 1H), 7.18 (s, 2H), 3.97-3.77 (m, 1H), 3.30-3.20 (m, 2H), 2.88 (s, 1H), 2.30-2.21 (m, 1H), 1.97 (d, J=11.4 Hz, 2H), 1.85 (d, J=11.0 Hz, 2H), 1.56 (q, J=11.6 Hz, 2H), 1.25-1.08 (m, 2H). MS (ES⁺): 408.1 [M+H]⁺.

### Example 30: 2-(thiazol-5-yl)-N-((1r,4r)-4-((2,2,2-trifluoroethyl)amino)cyclohexyl)pyrrolo[2,1-f][1,2,4]triazine-4-carboxamide

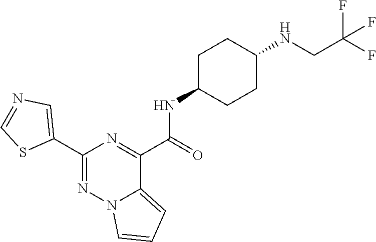 [SMILES: O=C(N[C@H]1CC[C@H](NCC(F)(F)F)CC1)c1nc(-c2cncs2)nn2cccc12]

Step 1: A mixture of ethyl 2-chloropyrrolo[2,1-f][1,2,4]triazine-4-carboxylate (200 mg, 0.886 mmol), 5-(4,4,5,5-tetramethyl-1,3,2-dioxaborolan-2-yl) thiazole (281 mg, 1.33 mmol), K₂CO₃ (245 mg, 1.77 mmol), Pd(dppf)Cl₂ (64.9 mg, 0.089 mmol) in H₂O (1 ml) and dioxane (3 ml) was degassed with N₂ (3×) and stirred at 100° C. for 3 h under N₂. The mixture was filtered and the filtrate was concentrated under reduced pressure. Purification of the residue by prep. HPLC (Xtimate C18 150*40 mm*10 μm, mobile phase A: 0.225% HCOOH in H₂O, mobile phase B: CH₃CN, 20 to 50% B) and lyophilization of the pure fractions gave 2-thiazol-5-ylpyrrolo[2,1-f][1,2,4]triazine-4-carboxylic acid (12 mg, 6%) as a yellow solid. MS (ES⁺): 246.9 [M+H]⁺.

Step 2: A solution of 2-thiazol-5-ylpyrrolo[2,1-f][1,2,4]triazine-4-carboxylic acid (12 mg, 0.049 mmol), (1r,4r)-N¹-(2,2,2-trifluoroethyl)cyclohexane-1,4-diamine hydrochloride (13.6 mg, 0.058 mmol) and triethylamine (14.8 mg, 0.146 mmol) in CH₂Cl₂ (1 ml) was dropwise treated with T3P (46.5 mg, 0.073 mmol, 50% in EtOAc) and stirred at 25° C. for 2 h. The mixture was diluted with H₂O (10 ml) and extracted with CH₂Cl₂ (3×10 ml). The combined organic layers were washed with brine (20 ml), dried over Na₂SO₄, filtered and concentrated under reduced pressure. The residue was purified by prep. HPLC (Phenomenex C18 75*30 mm*3 μm, mobile phase A: 0.05% NH₃ and 10 nM NH₄CO₃ in H₂O, mobile phase B: CH₃CN, 35 to 65% B). Lyophilization of the pure fractions gave the title compound (2 mg, 11%) as a yellow solid. ¹H NMR (400 MHz, DMSO-d₆): δ 9.27 (s, 1H), 9.07 (s, 1H), 8.86 (d, J=8.4 Hz, 1H), 8.29 (dd, J=1.4, 2.4 Hz, 1H), 7.51 (dd, J=1.4, 4.6 Hz, 1H), 7.17 (dd, J=2.6, 4.6 Hz, 1H), 3.91-3.78 (m, 1H), 3.30-3.21 (m, 2H), 2.47-2.40 (m, 1H), 2.26 (q, J=7.4 Hz, 1H), 1.97 (d, J=11.4 Hz, 2H), 1.86 (d, J=10.6 Hz, 2H), 1.62-1.51 (m, 2H), 1.20-1.09 (m, 2H). MS (ES⁺): 425.2 [M+H]⁺.

### Example 31: N-((1r,4r)-4-((2,2-difluoroethyl)amino)cyclohexyl)-2-(1H-imidazol-1-yl)pyrrolo[2,1-f][1,2,4]triazine-4-carboxamide

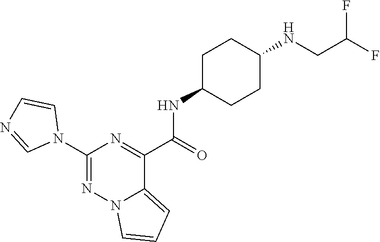 [SMILES: O=C(N[C@H]1CC[C@H](NCC(F)F)CC1)c1nc(-n2ccnc2)nn2cccc12]

A solution of 2-(1H-imidazol-1-yl)pyrrolo[2,1-f][1,2,4]triazine-4-carboxylic acid (Intermediate 5) (70 mg, 0.305 mmol), (1r,4r)-N¹-(2,2-difluoroethyl)cyclohexane-1,4-diamine (54.4 mg, 0.305 mmol), N,N-diisopropylethylamine (197 mg, 1.53 mmol) and CH₂Cl₂ (1 ml) was treated with T3P (292 mg, 0.458 mmol, 50% in EtOAc) and stirred at 25° C. for 30 min. The mixture was diluted with H₂O (20 ml) and extracted with CH₂Cl₂ (3×20 ml). The combined organic layers were dried over Na₂SO₄, filtered and concentrated under reduced pressure to give a residue, which was purified by prep. HPLC (Phenomenex C18 75*30 mm*3 μm, mobile phase A: 0.05% NH₃ and 10 nM NH₄CO₃ in H₂O, mobile phase B: CH₃CN, 24 to 54%). Lyophilization of the pure fractions gave the title compound (28 mg, 23%) as a yellow solid. ¹H NMR (400 MHz, DMSO-d₆): δ 9.00-8.91 (m, 2H), 8.35-8.27 (m, 1H), 8.09-8.02 (m, 1H), 7.59 (dd, J=1.4, 4.6 Hz, 1H), 7.23-7.14 (m, 2H), 6.13-5.79 (m, 1H), 3.92-3.80 (m, 1H), 2.97-2.87 (m, 2H), 2.46-2.39 (m, 1H), 2.01-1.93 (m, 2H), 1.90-1.80 (m, 3H), 1.62-1.52 (m, 2H), 1.18-1.08 (m, 2H). MS (ES⁺): 390.2 [M+H]⁺.

### Example 32: N-((1S,4r)-4-((S)-3-fluoropyrrolidin-1-yl)cyclohexyl)-2-(1H-imidazol-1-yl)pyrido[2,3-d]pyrimidine-4-carboxamide

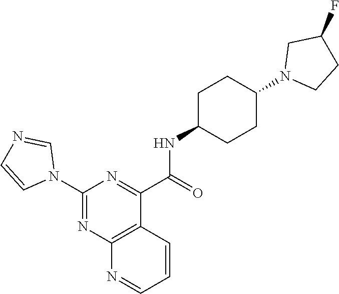 [SMILES: O=C(N[C@H]1CC[C@H](N2CC[C@H](F)C2)CC1)c1nc(-n2ccnc2)nc2ncccc12]

Following the procedure as described for Example 29 using 2-(1H-imidazol-1-yl)pyrido[2,3-d]pyrimidine-4-carboxylic acid (Intermediate 2) (50 mg, 0.207 mmol) and 4-[(3S)-3-fluoropyrrolidin-1-yl]cyclohexanamine (46.3 mg, 0.249 mmol), gave the title compound (1.6 mg, 1.9% yield) as a yellow solid. ¹H NMR (400 MHz, DMSO-d₆) 9.35-9.26 (m, 2H), 9.09 (d, J=8.4 Hz, 1H), 8.99 (s, 1H), 8.22 (s, 1H), 7.83-7.71 (m, 1H), 7.23 (s, 1H), 5.27-5.09 (m, 1H), 3.94-3.82 (m, 1H), 2.93-2.82 (m, 2H), 2.42-2.34 (m, 1H), 2.12-1.85 (m, 8H), 1.60-1.48 (m, 2H), 1.31-1.22 (m, 2H). MS ES⁺: 409.5. SFC: Rₜ=2.001 min, 96.24%.

### Example 33: N-((1r,4r)-4-((2,2-difluoroethyl)amino)cyclohexyl)-2-(1H-imidazol-1-yl)pyrido[2,3-d]pyrimidine-4-carboxamide

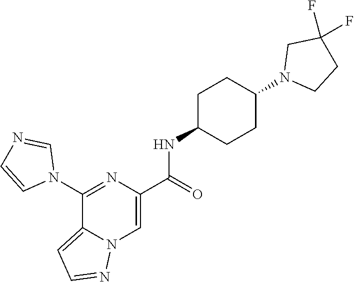 [SMILES: O=C(N[C@H]1CC[C@H](N2CCC(F)(F)C2)CC1)c1cn2nccc2c(-n2ccnc2)n1]

Following the procedure as described for Example 29 using 2-(1H-imidazol-1-yl)pyrido[2,3-d]pyrimidine-4-carboxylic acid (Intermediate 2) (5 mg, 0.021 mmol) and (1r,4r)-N¹-(2,2-difluoroethyl)cyclohexane-1,4-diamine (3.69 mg, 0.021 mmol) gave the title compound (2.6 mg, 29% yield) as a white solid. ¹H NMR (400 MHz, DMSO-d₆) 9.37-9.26 (m, 2H), 9.08 (br d, J=8.5 Hz, 1H), 8.99 (s, 1H), 8.23 (s, 1H), 7.78 (dd, J=4.5, 8.3 Hz, 1H), 7.23 (s, 1H), 5.95 (tt, J=4.3, 56.6 Hz, 1H), 3.95-3.80 (m, 2H), 2.92 (br t, J=15.9 Hz, 2H), 2.43 (br s, 1H), 1.94 (br t, J=14.6 Hz, 4H), 1.58-1.45 (m, 2H), 1.23-1.12 (m, 2H). MS ES⁺: 409.5. SFC: Rₜ=1.017 min, 97.38%.

### Example 34: N-((1r,4r)-4-(3,3-difluoropyrrolidin-1-yl)cyclohexyl)-4-(1H-imidazol-1-yl)pyrazolo[1,5-a]pyrazine-6-carboxamide

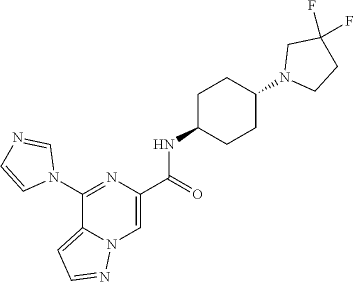 [SMILES: O=C(N[C@H]1CC[C@H](N2CCC(F)(F)C2)CC1)c1cn2nccc2c(-n2ccnc2)n1]

Following the procedure as described for Example 31 using 4-(1H-imidazol-1-yl)pyrazolo[1,5-a]pyrazine-6-carboxylic acid (Intermediate 10) (30 mg, 0.131 mmol) and (1r,4r)-4-(3,3-difluoropyrrolidin-1-yl)cyclohexan-1-amine (32.1 mg, 0.157 mmol) gave the title compound (6.98 mg, 13% yield) as a white solid. ¹H NMR (400 MHz, DMSO-d₆) 9.10 (d, J=0.6 Hz, 1H), 8.99 (s, 1H), 8.49-8.43 (m, 2H), 8.40 (d, J=1.3 Hz, 1H), 7.72-7.50 (m, 1H), 7.27 (s, 1H), 3.92-3.80 (m, 1H), 3.17 (d, J=5.1 Hz, 1H), 2.95 (br t, J=13.8 Hz, 2H), 2.76 (t, J=6.9 Hz, 2H), 2.26-2.18 (m, 2H), 1.97 (br d, J=11.8 Hz, 2H), 1.85 (br d, J=10.9 Hz, 2H), 1.57 (br d, J=12.9 Hz, 2H), 1.23 (br s, 2H). MS ES⁺: 416.2. SFC: Rₜ=2.18 min, 100%.

### Example 35: N-((1R,4r)-4-((R)-3-fluoropyrrolidin-1-yl)cyclohexyl)-4-(1H-imidazol-1-yl)pyrazolo[1,5-a]pyrazine-6-carboxamide

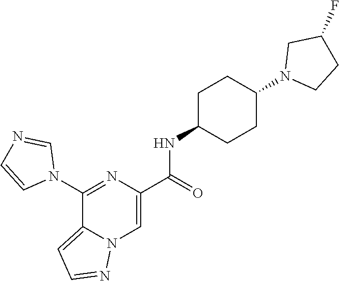 [SMILES: O=C(N[C@H]1CC[C@H](N2CC[C@@H](F)C2)CC1)c1cn2nccc2c(-n2ccnc2)n1]

A mixture of 6-chloro-4-(1H-imidazol-1-yl)pyrazolo[1,5-a]pyrazine (Intermediate 10, Step 1) (200 mg, 0.911 mmol), (1R,4r)-4-((R)-3-fluoropyrrolidin-1-yl)cyclohexan-1-amine (254 mg, 1.37 mmol), triethylamine (921 mg, 9.11 mmol), DPPP (188 mg, 455 mmol) and Pd(OAc)₂ (40.9 mg, 0.182 mmol) in DMF (2 mL) was degassed and purged with CO (3×), and then the mixture was stirred at 80° C. for 72 hours under CO atmosphere (50 psi). The reaction mixture was concentrated under reduced pressure to remove the solvent to give a residue. The residue was purified by prep. HPLC (Column: Xtimate C18 150×40 mm×10 μm, Mobile Phase A: water (NH₄OAc), Mobile Phase B: acetonitrile, Flow rate: 25 mL/min, gradient condition from 0% B to 40%). The pure fractions were collected and the volatiles were removed under vacuum. The residue was partitioned between acetonitrile (2 mL) and water (10 mL) and the mixture was lyophilized to dryness to give the title compound (3.2 mg, 0.85% yield) as a white solid. ¹H NMR (400 MHz, DMSO-d₆) 9.12-8.97 (m, 2H), 8.48 (d, J=8.6 Hz, 1H), 8.42 (d, J=18.1 Hz, 2H), 7.59 (s, 1H), 7.27 (s, 1H), 5.29-5.07 (m, 1H), 3.92-3.82 (m, 1H), 2.92-2.80 (m, 2H), 2.74-2.61 (m, 2H), 2.40-2.31 (m, 1H), 2.19-2.02 (m, 2H), 1.99 (d, J=13.0 Hz, 2H), 1.85 (d, J=13.0 Hz, 2H), 1.57 (q, J=11.7 Hz, 2H), 1.33-1.22 (m, 2H). MS ES⁺: 397.9.

### Example 36: N-((1S,4r)-4-((S)-3-fluoropyrrolidin-1-yl)cyclohexyl)-4-(1H-imidazol-1-yl)pyrazolo[1,5-a]pyrazine-6-carboxamide

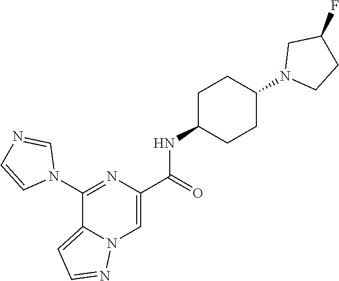 [SMILES: O=C(N[C@H]1CC[C@H](N2CC[C@H](F)C2)CC1)c1cn2nccc2c(-n2ccnc2)n1]

Following the procedure as described for Example 35 using 6-chloro-4-(1H-imidazol-1-yl)pyrazolo[1,5-a]pyrazine (Intermediate 10, Step 1) (220 mg, 1.00 mmol) and (1S,4r)-4-((S)-3-fluoropyrrolidin-1-yl)cyclohexan-1-amine (373 mg, 2.00 mmol) gave the title compound (25.24 mg, 6.3% yield) as a white solid. ¹H NMR (400 MHz, DMSO-d₆) 9.10 (s, 1H), 9.01 (s, 1H), 8.49 (d, J=8.6 Hz, 1H), 8.45 (d, J=2.4 Hz, 1H), 8.42 (s, 1H), 7.60 (d, J=1.9 Hz, 1H), 7.28 (s, 1H), 5.31-5.06 (m, 1H), 3.93-3.78 (m, 1H), 2.93-2.79 (m, 2H), 2.75-2.58 (m, 1H), 2.41-2.32 (m, 1H), 2.16-1.92 (m, 5H), 1.87-1.82 (m, 2H), 1.64-1.52 (m, 2H), 1.33-1.22 (m, 2H). MS ES⁺: 398.1. SFC: Rₜ=2.828 min, 100%.

### Example 37: N-((1r,4r)-4-((2,2-difluoroethyl)amino)cyclohexyl)-4-(1H-imidazol-1-yl)pyrazolo[1,5-a]pyrazine-6-carboxamide

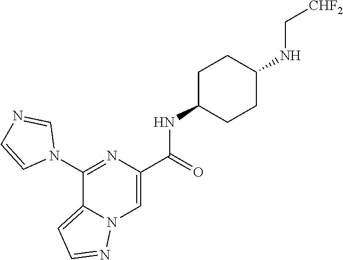 [SMILES: O=C(N[C@H]1CC[C@H](NCC(F)F)CC1)c1cn2nccc2c(-n2ccnc2)n1]

Following the procedure as described for Example 35 using 6-chloro-4-(1H-imidazol-1-yl)pyrazolo[1,5-a]pyrazine (Intermediate 10, Step 1) (200 mg, 0.911 mmol) and (1r,4r)-N¹-(2,2-difluoroethyl)cyclohexane-1,4-diamine (325 mg, 1.82 mmol) gave the title compound (6.06 mg, 1.7% yield) as a white solid. ¹H NMR (400 MHz, DMSO-d₆) 9.11 (s, 1H), 9.00 (s, 1H), 8.50-8.43 (m, 2H), 8.41 (s, 1H), 7.60 (d, J=1.8 Hz, 1H), 7.28 (s, 1H), 5.96 (, J=4.4, 56.6 Hz, 1H), 3.92-3.80 (m, 1H), 2.98-2.88 (m, 2H), 2.47-2.39 (m, 1H), 1.96 (d, J=12.8 Hz, 2H), 1.84 (d, J=9.8 Hz, 2H), 1.62-1.51 (m, 2H), 1.24 (s, 1H), 1.17-1.09 (m, 2H). MS ES⁺: 389.9. SFC: Rₜ=1.249 min, 100%.

### Example 38: N-((1r,4r)-4-((2,2-difluoroethyl)amino)cyclohexyl)-8-(1H-imidazol-1-yl) imidazo[1,2-a]pyrazine-6-carboxamide

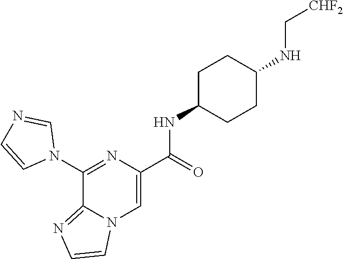 [SMILES: O=C(N[C@H]1CC[C@H](NCC(F)F)CC1)c1cn2ccnc2c(-n2ccnc2)n1]

Following the procedure as described for Example 29 using 8-(1H-imidazol-1-yl) imidazo[1,2-a]pyrazine-6-carboxylic acid (Intermediate 9) (30.0 mg, 0.131 mmol) and (1r,4r)-N¹-(2,2-difluoroethyl)cyclohexane-1,4-diamine (23.3 mg, 0.131 mmol) gave the title compound (4.13 mg, 7% yield) as a white solid. ¹H NMR (400 MHz, DMSO-d₆) 9.85-9.60 (m, 1H), 9.29 (s, 1H), 9.01-8.96 (m, 1H), 8.95-8.87 (m, 1H), 8.62-8.52 (m, 1H), 8.45 (s, 1H), 8.02 (s, 1H), 7.57-7.32 (m, 1H), 6.70-6.20 (m, 1H), 3.96-3.83 (m, 1H), 3.77-3.49 (m, 1H), 3.14-3.06 (m, 2H), 2.21-2.12 (m, 2H), 1.99-1.92 (m, 2H), 1.77-1.70 (m, 1H), 1.66-1.57 (m, 2H), 1.56-1.53 (m, 1H), 1.52-1.49 (m, 1H). MS ES⁺: 390.1. SFC: Rₜ=1.851 min, 100%.

### Example 39: N-((1R,4r)-4-((R)-3-fluoropyrrolidin-1-yl)cyclohexyl)-8-(1H-imidazol-1-yl) imidazo[1,2-a]pyrazine-6-carboxamide

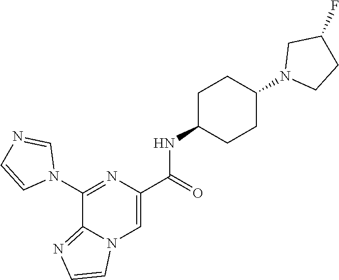 [SMILES: O=C(N[C@H]1CC[C@H](N2CC[C@@H](F)C2)CC1)c1cn2ccnc2c(-n2ccnc2)n1]

A mixture of 6-bromo-8-(1H-imidazol-1-yl) imidazo[1,2-a]pyrazine (Intermediate 9, Step 1) (200 mg, 0.757 mmol), (1R,4r)-4-((R)-3-fluoropyrrolidin-1-yl)cyclohexan-1-amine (169 mg, 0.909 mmol), Pd(OAc)₂ (3.40 mg, 0.015 mmol), Xantphos (8.76 mg, 0.015 mmol) and Na₂CO₃ (120 mg, 1.14 mmol) in toluene (0.5 mL) was degassed and purged with CO (3×), and then the mixture was stirred at 80° C. for 15 hours under CO (15 psi) atmosphere. The mixture was concentrated under reduced pressure to give a residue. The residue was purified by prep. HPLC (Column: Phenomenex C18 150×30 mm×10 μm, Mobile Phase A: water (HCOOH), Mobile Phase B: acetonitrile, Flow rate: 25 mL/min, gradient condition from 0% B to 30%). The pure fractions were collected and the volatiles were removed under vacuum. The residue was partitioned between acetonitrile (2 mL) and water (10 mL) and the mixture was lyophilized to dryness to give the title compound (62.33 mg, 18% yield) as a white solid. ¹H NMR (400 MHz, DMSO-d₆) 9.47 (s, 1H), 9.22 (s, 1H), 8.85 (t, J=1.3 Hz, 1H), 8.49 (d, J=8.8 Hz, 1H), 8.42 (d, J=1.0 Hz, 1H), 8.17 (s, 0.2H), 7.98 (d, J=1.0 Hz, 1H), 7.23 (s, 1H), 5.29-5.07 (m, 1H), 3.86 (dtd, J=4.3, 7.9, 15.5 Hz, 1H), 2.89-2.79 (m, 2H), 2.75-2.60 (m, 1H), 2.44-2.35 (m, 1H), 2.09-1.94 (m, 4H), 1.88-1.78 (m, 3H), 1.66-1.52 (m, 2H), 1.35-1.20 (m, 2H). MS ES⁺: 398.2. SFC: Rₜ=2.287 min, 98.64%.

### Example 40: N-((1S,4r)-4-((S)-3-fluoropyrrolidin-1-yl)cyclohexyl)-8-(1H-imidazol-1-yl) imidazo[1,2-a]pyrazine-6-carboxamide

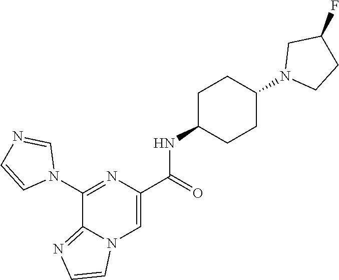 [SMILES: O=C(N[C@H]1CC[C@H](N2CC[C@H](F)C2)CC1)c1cn2ccnc2c(-n2ccnc2)n1]

Following the procedure as described for Example 39 using 6-bromo-8-(1H-imidazol-1-yl) imidazo[1,2-a]pyrazine (Intermediate 9, Step 1) (200 mg, 0.757 mmol) and (1S,4r)-4-((S)-3-fluoropyrrolidin-1-yl)cyclohexan-1-amine (169 mg, 0.909 mmol) gave the title compound (20.87 mg, 7% yield) as a white solid. ¹H NMR (400 MHz, DMSO-d₆) 9.48 (s, 1H), 9.22 (s, 1H), 8.85 (s, 1H), 8.49 (d, J=8.8 Hz, 1H), 8.42 (d, J=0.8 Hz, 1H), 7.98 (d, J=0.9 Hz, 1H), 7.23 (s, 1H), 5.31-5.06 (m, 1H), 3.94-3.80 (m, 1H), 2.88-2.80 (m, 2H), 2.75-2.60 (m, 1H), 2.43-2.34 (m, 1H), 2.10-1.96 (m, 4H), 1.91-1.76 (m, 3H), 1.67-1.53 (m, 2H), 1.35-1.21 (m, 2H). MS ES⁺: 398.2. SFC: Rₜ=2.287 min, 98.64%.

### Example 41: N-((1r,4r)-4-(3,3-difluoropyrrolidin-1-yl)cyclohexyl)-8-(1H-imidazol-1-yl) imidazo[1,2-a]pyrazine-6-carboxamide

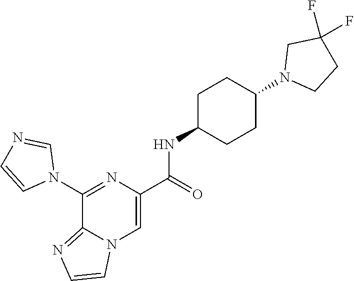 [SMILES: O=C(N[C@H]1CC[C@H](N2CCC(F)(F)C2)CC1)c1cn2ccnc2c(-n2ccnc2)n1]

Following the procedure as described for Example 39 using 6-bromo-8-(1H-imidazol-1-yl) imidazo[1,2-a]pyrazine (Intermediate 9, Step 1) (50 mg, 0.189 mmol) and (1r,4r)-4-(3,3-difluoropyrrolidin-1-yl)cyclohexan-1-amine (38.7 mg, 0.189 mmol) gave the title compound (21.22 mg, 26% yield) as a white solid. ¹H NMR (400 MHz, DMSO-d₆) 9.46 (s, 1H), 9.21 (s, 1H), 8.83 (t, J=1.3 Hz, 1H), 8.50 (d, J=8.8 Hz, 1H), 8.41 (d, J=1.1 Hz, 1H), 7.97 (d, J=1.0 Hz, 1H), 7.23 (s, 1H), 3.85 (tdd, J=4.0, 7.7, 11.7 Hz, 1H), 2.95 (t, J=13.8 Hz, 2H), 2.75 (t, J=6.9 Hz, 2H), 2.27-2.10 (m, 3H), 1.97 (br d, J=11.8 Hz, 2H), 1.84 (br d, J=10.6 Hz, 2H), 1.64-1.53 (m, 2H), 1.29-1.20 (m, 2H). MS ES⁺: 416.2. SFC: Rt=2.554 min, 100%.

### Example 42: 2-(5-methyl-1H-imidazol-1-yl)-N-((1r,4r)-4-((2,2,2-trifluoroethyl)amino)cyclohexyl)pyrrolo[2,1-f][1,2,4]triazine-4-carboxamide

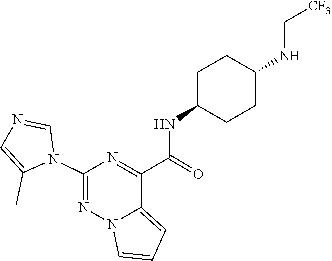 [SMILES: Cc1cncn1-c1nc(C(=O)N[C@H]2CC[C@H](NCC(F)(F)F)CC2)c2cccn2n1]

Following the procedure as described for Example 29 using 2-(5-methyl-1H-imidazol-1-yl)pyrrolo[2,1-f][1,2,4]triazine-4-carboxylic acid (Intermediate 11) (66 mg, 0.271 mmol) and N⁴-(2,2,2-trifluoroethyl)cyclohexane-1,4-diamine hydrochloride (75.8 mg, 0.326 mmol) gave the title compound 2 (2.49 mg, 2.2% yield) as a yellow solid. ¹H NMR (400 MHz, DMSO-d₆) 8.86 (d, J=8.6 Hz, 1H), 8.78 (d, J=1.0 Hz, 1H), 8.31 (dd, J=1.4, 2.5 Hz, 1H), 7.56 (dd, J=1.4, 4.6 Hz, 1H), 7.19 (dd, J=2.5, 4.6 Hz, 1H), 6.87 (s, 1H), 3.89-3.80 (m, 1H), 3.27 (br s, 2H), 2.54 (d, J=0.9 Hz, 3H), 2.45-2.40 (m, 2H), 1.99-1.93 (m, 2H), 1.87-1.81 (m, 2H), 1.49-1.47 (m, 2H), 0.94-0.92 (m, 2H). MS ES⁺: 422.2. SFC: Rₜ=0.673 min, 99.55%.

### Example 43: 2-(1H-imidazol-1-yl)-N-((1r,4r)-4-(pyrrolidin-1-yl)cyclohexyl)pyrrolo[2,1-f][1,2,4]triazine-4-carboxamide

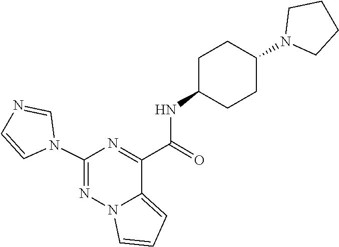 [SMILES: O=C(N[C@H]1CC[C@H](N2CCCC2)CC1)c1nc(-n2ccnc2)nn2cccc12]

Step 1: A solution of 4-(dibenzylamino)cyclohexanone (2 g, 6.82 mmol) and pyrrolidine (582 mg, 8.18 mmol) in dichloromethane (30 mL) was treated with AcOH (409 mg, 6.82 mmol) and NaBH(OAc)₃ (2.17 g, 10.22 mmol). The mixture was stirred at 25° C. for 12 hours. The mixture was concentrated under reduced pressure to give a residue. The residue was purified by flash silica gel chromatography (ISCO®; 20 g SepaFlash® Silica Flash Column, eluent of 0~10% MeOH/dichloromethane @20 mL/min). Then the product was further purified by prep. HPLC (Column: Welch Ultimate XB-CN 250×50 mm×10 μm, Mobile Phase A: heptane-EtOH (0.1% NH₃H₂O), Mobile Phase B: acetonitrile, Flow rate: 25 mL/min, gradient condition from 0% B to 16%). The pure fractions were collected and the volatiles were removed under vacuum. The residue was partitioned between acetonitrile (2 mL) and water (10 mL) and the mixture was lyophilized to dryness to give (1r,4r)-N,N-dibenzyl-4-(pyrrolidin-1-yl)cyclohexan-1-amine (364 mg, 1.04 mmol, 15% yield) as a white solid. ¹H NMR (400 MHz, DMSO-d₆) 7.44-7.09 (m, 10H), 3.56 (s, 4H), 2.61 (s, 4H), 2.45-2.31 (m, 1H), 2.17 (s, 1H), 1.98 (d, J=11.6 Hz, 2H), 1.83 (d, J=11.5 Hz, 2H), 1.66 (s, 4H), 1.46-1.32 (m, 2H), 1.07 (q, J=11.2 Hz, 2H).

Step 2: A mixture of (1r,4r)-N,N-dibenzyl-4-(pyrrolidin-1-yl)cyclohexan-1-amine (364 mg, 1.04 mmol), Pd(OH) 2 (367 mg, 0.522 mmol, 20% purity) in EtOH (15 mL) was degassed and purged with H₂ (3×), and then the mixture was stirred at 50° C. for 12 hours under H₂ (50 psi) atmosphere. The mixture was concentrated under reduced pressure to give crude (1r,4r)-4-(pyrrolidin-1-yl)cyclohexan-1-amine (108 mg, 0.642 mmol, 61% yield) as a white solid, which was used in the next step without further purification. MS ES⁺: 169.4.

Step 3: A solution of 2-chloropyrrolo[2,1-f][1,2,4]triazine-4-carboxylic acid (Intermediate 12) (106 mg, 0.535 mmol) and (1r,4r)-4-(pyrrolidin-1-yl)cyclohexan-1-amine (108 mg, 0.642 mmol) in DMF (5 mL) was treated with HATU (244 mg, 0.642 mmol) and triethylamine (271 mg, 2.67 mmol). The mixture was stirred at 25° C. for 12 hours. The mixture was concentrated under reduced pressure to give a residue. The residue was purified by flash silica gel chromatography (ISCO®; 4 g SepaFlash® Silica Flash Column, eluent of 0~5% EtOAc in petroleum ether, gradient @20 mL/min) to give crude product, which was purified by prep. HPLC (Column: Phenomenex C18 100×30 mm×10 μm, Mobile Phase A: water (HCOOH), Mobile Phase B: acetonitrile, Flow rate: 25 mL/min, gradient condition from 0% B to 60%). The pure fractions were collected and the volatiles were removed under vacuum. The residue was partitioned between acetonitrile (2 mL) and water (10 mL) and the mixture was lyophilized to dryness to give 2-chloro-N-((1r,4r)-4-(pyrrolidin-1-yl)cyclohexyl)pyrrolo[2,1-f][1,2,4]triazine-4-carboxamide (53 mg, 0.152 mmol, 28% yield) as a yellow solid. ¹H NMR (400 MHz, DMSO-d₆) 8.93 (d, J=8.4 Hz, 1H), 8.36 (s, 1H), 7.51 (d, J=3.9 Hz, 1H), 7.24 (dd, J=2.5, 4.6 Hz, 1H), 3.89-3.83 (m, 1H), 3.10 (s, 4H), 2.15 (s, 2H), 1.97-1.85 (m, 7H), 1.56 (t, J=9.4 Hz, 4H).

Step 4: A solution of 2-chloro-N-((1r,4r)-4-(pyrrolidin-1-yl)cyclohexyl)pyrrolo[2,1-f][1,2,4]triazine-4-carboxamide (35 mg, 0.101 mmol), DIPEA (39.0 mg, 0.302 mmol) and DMF (0.5 mL) was treated with 1H-imidazole (13.7 mg, 0.201 mmol). The mixture was stirred at 130° C. for 12 hours. The reaction mixture was extracted with EtOAc (3×10 mL). The combined organic layers were dried over Na₂SO₄, filtered and concentrated under reduced pressure to give a residue. The residue was purified by prep. HPLC (Column: Welch Xtimate C18 150×30 mm×5 μm, Mobile Phase A: water (NH₃H₂O+NH₄HCO₃), Mobile Phase B: acetonitrile, Flow rate: 25 mL/min, gradient condition from 22% B to 62%). The pure fractions were collected and the volatiles were removed under vacuum. The residue was partitioned between acetonitrile (2 mL) and water (10 mL) and the mixture was lyophilized to dryness to give the title compound (4.13 mg, 11% yield) as a yellow solid. ¹H NMR (400 MHz, DMSO-d₆) 9.01-8.89 (m, 2H), 8.33-8.28 (m, 1H), 8.09-8.03 (m, 1H), 7.64-7.55 (m, 1H), 7.23-7.14 (m, 2H), 3.92-3.82 (m, 1H), 2.52-2.52 (m, 4H), 2.07-1.94 (m, 4H), 1.89-1.83 (m, 2H), 1.68-1.65 (m, 3H), 1.62-1.53 (m, 2H), 1.34-1.27 (m, 2H). MS ES⁺: 380.1. SFC: Rₜ=2.205 min, 97.01%.

### Example 44: 2-(1H-imidazol-1-yl)-N-((1s,4s)-4-(pyrrolidin-1-yl)cyclohexyl)pyrrolo[2,1-f][1,2,4]triazine-4-carboxamide

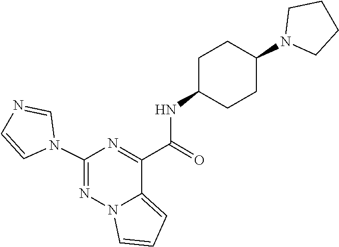 [SMILES: O=C(N[C@H]1CC[C@@H](N2CCCC2)CC1)c1nc(-n2ccnc2)nn2cccc12]

Step 1: A solution of 4-(dibenzylamino)cyclohexan-1-one (3.64 g, 12.4 mmol) and pyrrolidine (1.06 g, 14.89 mmol) in dichloromethane (30 mL) was treated with AcOH (745 mg, 12.41 mmol) and NaBH(OAc)₃ (3.94 g, 18.6 mmol). The mixture was stirred at 25° C. for 12 hours. The mixture was concentrated under reduced pressure to give a residue. The residue was purified by prep. HPLC (Column: Welch Ultimate XB-CN 250×50 mm×10 μm, Mobile Phase A: Heptane-EtOH (0.1% NH₃H₂O), Mobile Phase B: acetonitrile, Flow rate: 25 mL/min, gradient condition from 0% B to 16%). The pure fractions were collected and the volatiles were removed under vacuum. The residue was partitioned between acetonitrile (2 mL) and water (10 mL) and the mixture was lyophilized to dryness to give (1s,4s)-N,N-dibenzyl-4-(pyrrolidin-1-yl)cyclohexan-1-amine (1.92 g, 5.51 mmol, 44% yield) as a white solid. ¹H NMR (400 MHz, DMSO-d₆) 7.42-7.12 (m, 10H), 6.73-6.25 (m, 2H), 3.86-3.77 (m, 2H), 3.71-3.61 (m, 2H), 3.59-3.54 (m, 3H), 3.23-3.04 (m, 1H), 2.44 (br d, J=3.0 Hz, 1H), 2.04-1.81 (m, 2H), 1.80-1.64 (m, 4H), 1.62-1.47 (m, 3H), 1.38-1.19 (m, 2H).

Step 2: A mixture of (1s,4s)-N,N-dibenzyl-4-(pyrrolidin-1-yl)cyclohexan-1-amine (1.92 g, 5.51 mmol), Pd(OH) 2 (1.93 g, 2.75 mmol, 20% purity) in EtOH (15 mL) was degassed and purged with H₂ (3×), and then the mixture was stirred at 50° C. for 12 hours under H₂ (50 psi) atmosphere. The mixture was concentrated under reduced pressure to give (1s,4s)-4-(pyrrolidin-1-yl)cyclohexan-1-amine (542 mg, 3.22 mmol, 58% yield) as a clear liquid which was used in the next step without further purification. ¹H NMR (400 MHz, DMSO-d₆) 3.90 (s, 1H), 3.02 (s, 4H), 2.81-2.72 (m, 1H), 2.58 (s, 1H), 2.07 (t, J=5.8 Hz, 1H), 1.81-1.67 (m, 6H), 1.60-1.41 (m, 6H).

Step 3: A solution of 2-chloropyrrolo[2,1-f][1,2,4]triazine-4-carboxylic acid (Intermediate 12) (460 mg, 2.33 mmol) and (1s,4s)-4-(pyrrolidin-1-yl)cyclohexan-1-amine (399 mg, 2.37 mmol) in DMF (10 mL) was treated with triethylamine (1.20 g, 11.9 mmol) and HATU (1.08 g, 2.84 mmol). The mixture was stirred at 25° C. for 2 hours. The reaction mixture was diluted with EtOAc (100 mL) and washed with H₂O (3×30 mL) and brine (30 mL). The organic layer was dried over Na₂SO₄, filtered and concentrated under reduced pressure to give a residue. The residue was purified by prep. HPLC (Column: Welch Xtimate C18 150×30 mm×5 μm, Mobile Phase A: water (HCOOH), Mobile Phase B: acetonitrile, Flow rate: 25 mL/min, gradient condition from 0% B to 28%). The pure fractions were collected and the volatiles were removed under vacuum. The residue was partitioned between acetonitrile (2 mL) and water (10 mL) and the mixture was lyophilized to dryness to give 2-chloro-N-((1s,4s)-4-(pyrrolidin-1-yl)cyclohexyl)pyrrolo[2,1-f][1,2,4]triazine-4-carboxamide (18 mg, 0.044 mmol, 2% yield) as a yellow solid. MS ES⁺: 348.5.

Step 4: A solution of 2-chloro-N-((1,4s)-4-(pyrrolidin-1-yl)cyclohexyl)pyrrolo[2,1-f][1,2,4]triazine-4-carboxamide (15 mg, 0.043 mmol), DIPEA (16.72 mg, 0.129 mmol) and DMF (1.5 mL) was treated with 1H-imidazole (5.87 mg, 0.0862 mmol). The mixture and stirred at 130° C. for 12 hours. The reaction mixture was extracted with EtOAc (3×10 mL). The combined organic layers were dried over Na₂SO₄, filtered and concentrated under reduced pressure to give a residue. The residue was purified by prep. HPLC (Column: Welch Xtimate C18 150×30 mm×5 μm, Mobile Phase A: water (HCOOH), Mobile Phase B: acetonitrile, Flow rate: 25 mL/min, gradient condition from 0% B to 24%). The pure fractions were collected and the volatiles were removed under vacuum. The residue was partitioned between acetonitrile (2 mL) and water (10 mL) and the mixture was lyophilized to dryness to give the title compound (1.79 mg, 9% yield) as a yellow solid. ¹H NMR (400 MHz, DMSO-d₆) 9.09 (br d, J=8.4 Hz, 1H), 9.00 (s, 1H), 8.33-8.27 (m, 1H), 8.21 (s, 1H), 8.09 (s, 1H), 7.62-7.57 (m, 1H), 7.22-7.14 (m, 2H), 4.05-3.90 (m, 1H), 2.55 (br s, 4H), 2.22 (br s, 1H), 1.96-1.87 (m, 4H), 1.74 (br s, 4H), 1.61-1.51 (m, 4H). MS ES⁺: 380.1. SFC: Rₜ=1.425 min, 100%.

### Example 45: N-((1r,4r)-4-(3,3-difluoroazetidin-1-yl)cyclohexyl)-8-(1H-imidazol-1-yl)-[1,2,4]triazolo[1,5-a]pyrazine-6-carboxamide

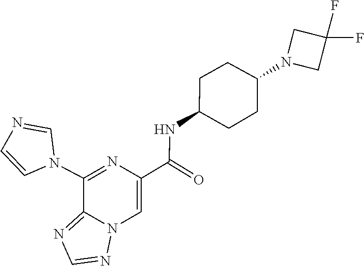 [SMILES: O=C(N[C@H]1CC[C@H](N2CC(F)(F)C2)CC1)c1cn2ncnc2c(-n2ccnc2)n1]

A mixture of 6-bromo-8-(1H-imidazol-1-yl)-[1,2,4]triazolo[1,5-a]pyrazine (Intermediate 14) (300 mg, 1.13 mmol), (1r,4r)-4-(3,3-difluoroazetidin-1-yl)cyclohexan-1-amine (Intermediate 13) (258 mg, 1.36 mmol), Pd(OAc)₂ (5.08 mg, 0.023 mmol), Xantphos (13.10 mg, 0.023 mmol) and Na₂CO₃ (179.93 mg, 1.70 mmol) in toluene (3 mL) was degassed and purged with CO (3×), and then the mixture was stirred at 80° C. for 12 hours under CO (50 psi) atmosphere. The mixture was poured into water (30 mL) and extracted with EtOAc (3×30 mL). The combined organic layers were concentrated to afford crude product which was purified by prep. HPLC (Column: Welch Xtimate C18 150×30 mm×5 μm, Mobile Phase A: water (HCOOH), Mobile Phase B: acetonitrile, Flow rate: 25 mL/min, gradient condition from 0% B to 20%). The pure fractions were collected and the volatiles were removed under vacuum. The residue was partitioned between acetonitrile (2 mL) and water (10 mL) and the mixture was lyophilized to dryness to give the title compound (88.51 mg, 17% yield) as a white solid. ¹H NMR (400 MHz, DMSO-d₆) 9.39 (s, 1H), 9.32 (s, 1H), 8.96 (s, 1H), 8.78 (t, J=1.3 Hz, 1H), 8.65 (d, J=8.6 Hz, 1H), 8.15 (s, 0.2H), 7.28 (s, 1H), 3.92-3.81 (m, 1H), 3.56 (t, J=12.3 Hz, 4H), 2.14 (br t, J=10.6 Hz, 1H), 1.86-1.79 (m, 4H), 1.64-1.51 (m, 2H), 1.16-1.06 (m, 2H). MS ES⁺: 402.4. SFC: Rₜ=2.028 min, 99.81%.

### Example 46: N-((1r,4r)-4-(3-fluoro-3-methylazetidin-1-yl)cyclohexyl)-8-(1H-imidazol-1-yl)-[1,2,4]triazolo[1,5-a]pyrazine-6-carboxamide

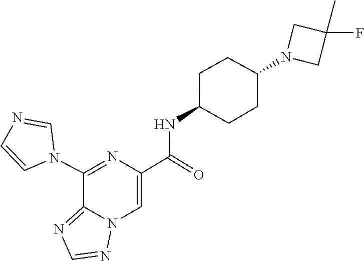 [SMILES: CC1(F)CN([C@H]2CC[C@H](NC(=O)c3cn4ncnc4c(-n4ccnc4)n3)CC2)C1]

Following the procedure as described for Example 45 using (1r,4r)-4-(3-fluoro-3-methylazetidin-1-yl)cyclohexan-1-amine (Intermediate 15) (120 mg, 0.644 mmol) and 6-bromo-8-(1H-imidazol-1-yl)-[1,2,4]triazolo[1,5-a]pyrazine (Intermediate 14) (205 mg, 0.773 mmol) gave the title compound (6.3 mg, 2.2% yield) as a yellow solid. ¹H NMR (400 MHz, DMSO-d₆) 11.69-11.40 (m, 1H), 10.43-10.32 (m, 1H), 9.56 (s, 1H), 9.12-9.06 (m, 2H), 9.05-8.96 (m, 1H), 7.82 (s, 1H), 4.43 (br d, J=10.8 Hz, 2H), 4.16 (br d, J=3.4 Hz, 2H), 2.48-2.34 (m, 1H), 2.07-1.90 (m, 4H), 1.74-1.62 (m, 4H), 1.57-1.43 (m, 3H). MS ES⁺: 399.4. SFC: Rₜ=2.011 min, 100%.

### Example 47: 8-(1H-imidazol-1-yl)-N-((1r,4r)-4-(methyl (2,2,2-trifluoroethyl)amino)cyclohexyl)-[1,2,4]triazolo[1,5-a]pyrazine-6-carboxamide

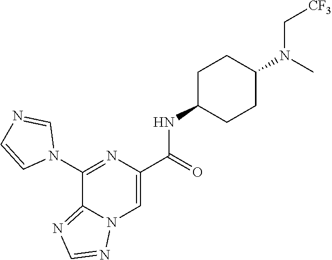 [SMILES: CN(CC(F)(F)F)[C@H]1CC[C@H](NC(=O)c2cn3ncnc3c(-n3ccnc3)n2)CC1]

Following the procedure as described for Example 45 using (1r,4r)-N¹-methyl-N¹-(2,2,2-trifluoroethyl)cyclohexane-1,4-diamine (prepared as described in WO2019/094641) (100 mg, 0.476 mmol) and 6-bromo-8-(1H-imidazol-1-yl)-[1,2,4]triazolo[1,5-a]pyrazine (Intermediate 14) (151 mg, 0.571 mmol), gave the title compound (11.01 mg, 5.5% yield) as a white solid. ¹H NMR (400 MHz, DMSO-d₆) 9.39 (s, 1H), 9.32 (s, 1H), 8.96 (s, 1H), 8.78 (s, 1H), 8.63 (d, J=8.6 Hz, 1H), 7.28 (s, 1H), 3.95-3.84 (m, 1H), 3.23-3.14 (m, 2H), 2.48 (br s, 1H), 2.39 (s, 3H), 1.91-1.77 (m, 4H), 1.66-1.54 (m, 2H), 1.45-1.35 (m, 2H). MS ES⁺: 423.4. SFC: Rₜ=1.811 min, 100%.

### Example 48: N-((1r,4r)-4-(3,3-difluoropyrrolidin-1-yl)cyclohexyl)-8-(1H-imidazol-1-yl)-[1,2,4]triazolo[1,5-a]pyrazine-6-carboxamide

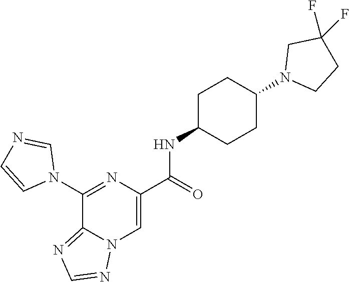 [SMILES: O=C(N[C@H]1CC[C@H](N2CCC(F)(F)C2)CC1)c1cn2ncnc2c(-n2ccnc2)n1]

Following the procedure as described for Example 45 using 6-bromo-8-(1H-imidazol-1-yl)-[1,2,4]triazolo[1,5-a]pyrazine (Intermediate 14) (388 mg, 1.46 mmol) and (1r,4r)-4-(3,3-difluoropyrrolidin-1-yl)cyclohexan-1-amine (250 mg, 1.22 mmol) gave the title compound (35.16 mg, 5.6% yield) as a white solid. ¹H NMR (400 MHz, DMSO-d₆) 9.40 (s, 1H), 9.33 (s, 1H), 8.97 (s, 1H), 8.80 (s, 1H), 8.66 (d, J=8.6 Hz, 1H), 7.29 (s, 1H), 3.95-3.80 (m, 1H), 2.96 (t, J=13.8 Hz, 2H), 2.76 (t, J=6.9 Hz, 2H), 2.29-2.06 (m, 3H), 1.98 (d, J=12.1 Hz, 2H), 1.85 (d, J=10.5 Hz, 2H), 1.70-1.50 (m, 2H), 1.35-1.18 (m, 2H). MS ES⁺: 417.2. SFC: Rₜ=2.213 min, 100%.

### Example 49: 8-(1H-imidazol-1-yl)-N-((1r,4r)-4-morpholinocyclohexyl)-[1,2,4]triazolo[1,5-a]pyrazine-6-carboxamide

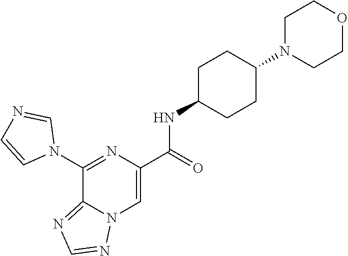 [SMILES: O=C(N[C@H]1CC[C@H](N2CCOCC2)CC1)c1cn2ncnc2c(-n2ccnc2)n1]

Following the procedure as described for Example 45 using 6-bromo-8-(1H-imidazol-1-yl)-[1,2,4]triazolo[1,5-a]pyrazine (Intermediate 14) (200 mg, 0.755 mmol) and (1r,4r)-4-morpholinocyclohexan-1-amine dihydrochloride (233 mg, 0.905 mmol) gave the title compound (6.41 mg, 2.1% yield) as a white solid. ¹H NMR (400 MHz, DMSO-d₆) 9.39 (s, 1H), 9.33 (s, 1H), 8.97 (s, 1H), 8.81-8.77 (m, 1H), 8.64 (d, J=8.6 Hz, 1H), 7.29 (s, 1H), 3.95-3.78 (m, 1H), 3.61-3.50 (m, 5H), 3.32 (br s, 2H), 2.22 (t, J=11.4 Hz, 1H), 1.97-1.76 (m, 5H), 1.65-1.51 (m, 2H), 1.39-1.25 (m, 2H). MS ES⁺: 397.2. SFC: Rₜ=1.199 min, 99.37%.

### Example 50: 8-(1H-imidazol-1-yl)-N-(1-(2,2,2-trifluoroethyl) piperidin-4-yl)-[1,2,4]triazolo[1,5-a]pyrazine-6-carboxamide

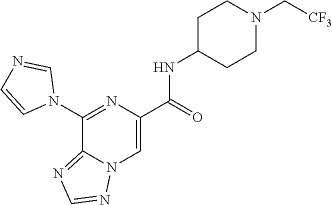 [SMILES: O=C(NC1CCN(CC(F)(F)F)CC1)c1cn2ncnc2c(-n2ccnc2)n1]

Step 1: A mixture of 6-bromo-8-(1H-imidazol-1-yl)-[1,2,4]triazolo[1,5-a]pyrazine (Intermediate 14) (300 mg, 1.13 mmol), tert-butyl 4-aminopiperidine-1-carboxylate (272 mg, 1.36 mmol), Pd(OAc)₂ (50.8 mg, 0.226 mmol), DPPP (233 mg, 0.566 mmol) and triethylamine (1.15 g, 11.32 mmol) in DMF (0.5 mL) was degassed and purged with CO (3×), and then the mixture was stirred at 80° C. for 12 hours under CO (50 psi) atmosphere. The reaction mixture was concentrated under reduced pressure to remove the solvent to give a residue. The residue was purified by prep. HPLC (Column: Phenomenex C18 150×40 mm×10 μm, Mobile Phase A: water (HCOOH), Mobile Phase B: MeOH, Flow rate: 25 mL/min, gradient condition from 26% B to 56%). The pure fractions were collected and the volatiles were removed under vacuum. The residue was partitioned between acetonitrile (2 mL) and water (10 mL) and the mixture was lyophilized to dryness to give tert-butyl 4-(8-(1H-imidazol-1-yl)-[1,2,4]triazolo[1,5-a]pyrazine-6-carboxamido) piperidine-1-carboxylate (283 mg, 0.686 mmol, 61% yield) as a yellow solid. ¹H NMR (400 MHz, DMSO-d₆) 9.39 (s, 1H), 9.35 (s, 1H), 8.97 (s, 1H), 8.79 (s, 1H), 8.71 (d, J=8.8 Hz, 1H), 7.29 (s, 1H), 4.21-3.95 (m, 3H), 3.01-2.70 (m, 2H), 1.84-1.72 (m, 2H), 1.71-1.56 (m, 2H), 1.42 (s, 9H).

Step 2: To a solution of tert-butyl 4-(8-(1H-imidazol-1-yl)-[1,2,4]triazolo[1,5-a]pyrazine-6-carboxamido) piperidine-1-carboxylate (283 mg, 0.686 mmol) in dichloromethane (3 mL) was added 4M HCl in dioxane (5 mL). The mixture was stirred at 25° C. for 1 hour. The reaction mixture was quenched with sat. NaHCO₃ (aq.) to pH=7-8 at 0° C. and concentrated under reduced pressure to give 8-(1H-imidazol-1-yl)-N-(piperidin-4-yl)-[1,2,4]triazolo[1,5-a]pyrazine-6-carboxamide (291 mg) as a white solid which was used without further purification. MS ES⁺: 312.9.

Step 3: A solution of 8-(1H-imidazol-1-yl)-N-(piperidin-4-yl)-[1,2,4]triazolo[1,5-a]pyrazine-6-carboxamide (291 mg, 0.932 mmol) and 2,2,2-trifluoroethyl trifluoromethanesulfonate (260 mg, 1.12 mmol) in acetonitrile (3 mL) was treated with DIPEA (361 mg, 2.80 mmol). The mixture was stirred at 70° C. for 2 hours. The mixture was concentrated under reduced pressure to give a residue. The residue was purified by prep. HPLC (Column: Phenomenex C18 150×40 mm×10 μm, Mobile Phase A: water (NH₃H₂O+NH₄HCO₃), Mobile Phase B: MeOH, Flow rate: 25 mL/min, gradient condition from 12% B to 52%). The pure fractions were collected and the volatiles were removed under vacuum. The residue was partitioned between acetonitrile (2 mL) and water (10 mL) and the mixture was lyophilized to dryness to give the title compound (113.3 mg, 31% yield) as a white solid. ¹H NMR (400 MHz, DMSO-d₆) 9.40 (s, 1H), 9.34 (s, 1H), 8.97 (s, 1H), 8.81 (t, J=1.3 Hz, 1H), 8.69 (d, J=8.9 Hz, 1H), 7.29 (s, 1H), 4.00-3.87 (m, 1H), 3.20 (q, J=10.1 Hz, 2H), 2.99 (d, J=11.6 Hz, 2H), 2.48-2.37 (m, 2H), 1.86 (dq, J=3.7, 12.1 Hz, 2H), 1.79-1.70 (m, 2H). MS ES⁺: 395.5. SFC: Rₜ=1.152 min, 100%.

### Example 51: N-((1r,4r)-4-(1,1-dioxidothiomorpholino)cyclohexyl)-8-(1H-imidazol-1-yl)-[1,2,4]triazolo[1,5-a]pyrazine-6-carboxamide

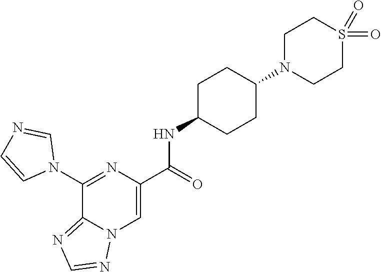 [SMILES: O=C(N[C@H]1CC[C@H](N2CCS(=O)(=O)CC2)CC1)c1cn2ncnc2c(-n2ccnc2)n1]

Following the procedure as described for Example 29 using 4-(4-aminocyclohexyl)thiomorpholine 1,1-dioxide hydrochloride (Intermediate 17) (70 mg, 0.260 mmol) and 8-(1H-imidazol-1-yl)-[1,2,4]triazolo[1,5-a]pyrazine-6-carboxylic acid (Intermediate 16) (299 mg, 1.30 mmol) gave the title compound (1.37 mg, 1.1% yield) as a pink solid. ¹H NMR (400 MHz, DMSO-d₆) 9.40 (s, 1H), 9.36-9.34 (m, 1H), 8.97 (s, 1H), 8.79 (t, J=1.3 Hz, 1H), 8.70 (d, J=8.5 Hz, 1H), 7.29 (d, J=0.8 Hz, 1H), 3.88 (br t, J=12.0 Hz, 2H), 3.60-3.53 (m, 2H), 3.31 (br s, 2H), 2.99 (br d, J=11.6 Hz, 2H), 2.83 (br d, J=13.4 Hz, 2H), 2.45 (br s, 2H), 2.02-1.98 (m, 2H), 1.69 (br d, J=10.3 Hz, 4H). MS ES⁺: 444.5. SFC: Rₜ=3.355 min, 96.5%.

### Example 52: N-((1r,4r)-4-((2,2-difluoroethyl)amino)cyclohexyl)-8-(1H-imidazol-1-yl)-[1,2,4]triazolo[1,5-a]pyrazine-6-carboxamide

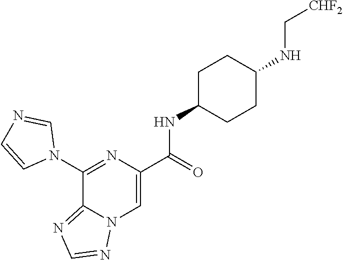 [SMILES: O=C(N[C@H]1CC[C@H](NCC(F)F)CC1)c1cn2ncnc2c(-n2ccnc2)n1]

Following the procedure as described for Example 29 using 8-(1H-imidazol-1-yl)-[1,2,4]triazolo[1,5-a]pyrazine-6-carboxylic acid (Intermediate 16) (95 mg, 0.413 mmol) and (1r,4r)-N¹-(2,2-difluoroethyl)cyclohexane-1,4-diamine (73.6 mg, 0.413 mmol) gave the title compound (5.29 mg, 3.3% yield) as a white solid. ¹H NMR (400 MHz, DMSO-d₆) 9.40 (s, 1H), 9.35 (s, 1H), 8.99 (s, 1H), 8.79 (s, 1H), 8.71 (br d, J=8.5 Hz, 1H), 7.30 (s, 1H), 6.48-6.12 (m, 1H), 3.93-3.85 (m, 2H), 2.12 (br d, J=10.1 Hz, 3H), 1.92 (br d, J=9.5 Hz, 3H), 1.70-1.53 (m, 3H), 1.44-1.33 (m, 2H). MS ES⁺: 391.4. SFC: Rt=1.106 min, 100%.

### Example 53: 8-(1H-imidazol-1-yl)-N-((1r,4r)-4-((1,1,1-trifluoro-2-methylpropan-2-yl)amino)cyclohexyl)-[1,2,4]triazolo[1,5-a]pyrazine-6-carboxamide

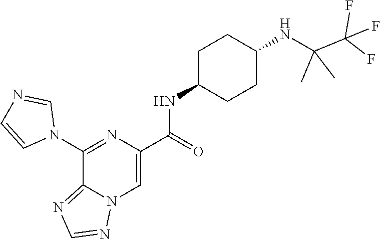 [SMILES: CC(C)(N[C@H]1CC[C@H](NC(=O)c2cn3ncnc3c(-n3ccnc3)n2)CC1)C(F)(F)F]

A solution of 8-(1H-imidazol-1-yl)-[1,2,4]triazolo[1,5-a]pyrazine-6-carboxylic acid (Intermediate 16) (150 mg, 0.652 mmol), (1r,4r)-N¹-(1,1,1-trifluoro-2-methylpropan-2-yl)cyclohexane-1,4-diamine hydrochloride (Intermediate 18) (339 mg, 1.30 mmol) and 2,4,6-tributyl-1,3,5,2,4,6-trioxatriphosphinane-2,4,6-trioxide (T4P) (704 mg, 0.977 mmol, 50% purity) in dichloromethane (2 mL) was treated with DIPEA (253 mg, 1.95 mmol). The mixture was stirred at 25° C. for 1 hour. The reaction mixture was diluted with dichloromethane (15 mL) and washed with H₂O (3×10 mL). The organic layer was dried over Na₂SO₄, filtered and concentrated under reduced pressure to give a residue. The residue was purified by prep. HPLC (Column: Xtimate C18 150×40 mm×10 μm, Mobile Phase A: water (NH₃H₂O+NH₄HCO₃), Mobile Phase B: acetonitrile, Flow rate: 25 mL/min, gradient condition from 26% B to 66%). The pure fractions were collected and the volatiles were removed under vacuum. The residue was partitioned between acetonitrile (2 mL) and water (10 mL) and the mixture was lyophilized to dryness to give the title compound (5.60 mg, 2% yield) as a white solid. ¹H NMR (400 MHz, DMSO-d₆) 9.42-9.35 (m, 1H), 9.34-9.30 (m, 1H), 8.97 (s, 1H), 8.84-8.74 (m, 1H), 8.64-8.53 (m, 1H), 7.31-7.24 (m, 1H), 3.96-3.77 (m, 1H), 2.73-2.58 (m, 1H), 1.88-1.79 (m, 4H), 1.70-1.54 (m, 2H), 1.33-1.13 (m, 9H). MS ES⁺: 437.2. SFC: Rₜ=1.652 min, 99.82%.

### Example 54: 8-(1H-imidazol-1-yl)-N-((1S,4s)-4-((R)-3-methoxypyrrolidin-1-yl)cyclohexyl)-[1,2,4]triazolo[1,5-a]pyrazine-6-carboxamide

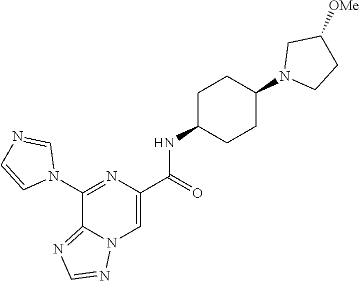 [SMILES: CO[C@@H]1CCN([C@H]2CC[C@@H](NC(=O)c3cn4ncnc4c(-n4ccnc4)n3)CC2)C1]

Following the procedure as described for Example 29 using (1S,4s)-4-((R)-3-methoxypyrrolidin-1-yl)cyclohexan-1-amine hydrochloride (Intermediate 19) (1 g, 4.26 mmol) and 8-(1H-imidazol-1-yl)-[1,2,4]triazolo[1,5-a]pyrazine-6-carboxylic acid (Intermediate 16) (980 mg, 4.26 mmol) gave the title compound (19.17 mg, 0.9% yield) as a white solid. ¹H NMR (400 MHz, DMSO-d₆) 9.42 (s, 1H), 9.32 (s, 1H), 9.02-8.91 (m, 1H), 8.88-8.77 (m, 2H), 7.27 (s, 1H), 4.01-3.86 (m, 2H), 3.18 (s, 3H), 2.78 (dd, J=6.6, 9.9 Hz, 1H), 2.57 (br d, J=7.4 Hz, 1H), 2.46 (br d, J=2.6 Hz, 1H), 2.15 (br s, 1H), 2.09-1.79 (m, 6H), 1.75-1.64 (m, 1H), 1.59-1.45 (m, 4H). MS ES⁺: 410.5. SFC: Rₜ=1.239 min, 91.05%.

### Example 55: 8-(1H-imidazol-1-yl)-N-((1s,4s)-4-thiomorpholinocyclohexyl)-[1,2,4]triazolo[1,5-a]pyrazine-6-carboxamide

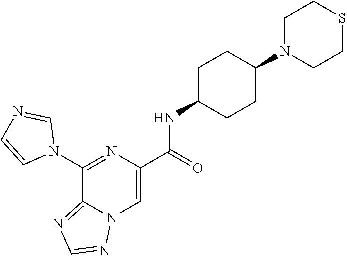 [SMILES: O=C(N[C@H]1CC[C@@H](N2CCSCC2)CC1)c1cn2ncnc2c(-n2ccnc2)n1]

Following the procedure as described for Example 29 using 4-thiomorpholinocyclohexan-1-amine hydrochloride (300 mg, 1.27 mmol) and 8-(1H-imidazol-1-yl)-[1,2,4]triazolo[1,5-a]pyrazine-6-carboxylic acid (Intermediate 16) (292 mg, 1.27 mmol) gave the title compound (1.20 mg, 0.24% yield) as a yellow solid. ¹H NMR (400 MHz, DMSO-d₆) 9.43-9.25 (m, 1H), 9.01-8.90 (m, 1H), 8.84-8.73 (m, 1H), 8.55 (s, 1H), 7.37-7.15 (m, 1H), 3.90-3.80 (m, 2H), 2.78 (br d, J=1.1 Hz, 3H), 2.58 (br s, 4H), 2.39-2.25 (m, 2H), 1.91-1.85 (m, 1H), 1.81-1.74 (m, 1H), 1.69-1.53 (m, 2H), 1.51-1.36 (m, 2H), 1.32-1.12 (m, 2H). MS ES⁺: 413.4. SFC: Rₜ=2.607 min, 95.46%.

### Example 56: 8-(1H-imidazol-1-yl)-N-((1r,4r)-4-(3-(trifluoromethyl) azetidin-1-yl)cyclohexyl)-[1,2,4]triazolo[1,5-a]pyrazine-6-carboxamide

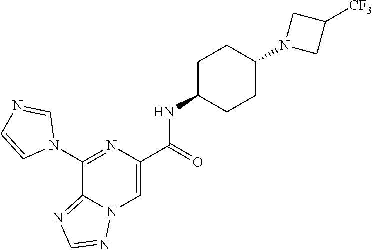 [SMILES: O=C(N[C@H]1CC[C@H](N2CC(C(F)(F)F)C2)CC1)c1cn2ncnc2c(-n2ccnc2)n1]

Following the procedure as described for Example 29 using (1r,4r)-4-(3-(trifluoromethyl) azetidin-1-yl)cyclohexan-1-amine (Intermediate 20) (50 mg, 0.225 mmol) and 8-(1H-imidazol-1-yl)-[1,2,4]triazolo[1,5-a]pyrazine-6-carboxylic acid (Intermediate 16) (51.8 mg, 0.225 mmol) gave the title compound (2.35 mg, 2.2% yield) as a white solid. ¹H NMR (400 MHz, DMSO-d₆) 9.44-9.26 (m, 2H), 8.97 (s, 1H), 8.79 (s, 1H), 8.22 (s, 1H), 7.29 (s, 1H), 3.89-3.82 (m, 1H), 3.40 (br s, 2H), 3.33-3.30 (m, 1H), 3.13 (br t, J=6.3 Hz, 2H), 2.02 (br t, J=10.8 Hz, 1H), 1.81 (br s, 4H), 1.57 (q, J=11.8 Hz, 2H), 1.01 (br d, J=11.8 Hz, 2H). MS ES⁺: 435.2. SFC: Rₜ=1.85 min, 100%.

### Example 57: N-((1r,4r)-4-((2,2-difluoropropyl)amino)cyclohexyl)-8-(1H-imidazol-1-yl)-[1,2,4]triazolo[1,5-a]pyrazine-6-carboxamide

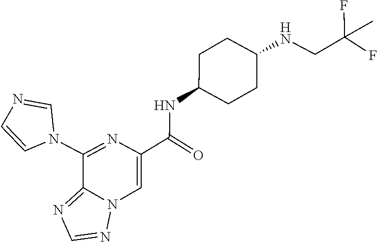 [SMILES: CC(F)(F)CN[C@H]1CC[C@H](NC(=O)c2cn3ncnc3c(-n3ccnc3)n2)CC1]

Following the procedure as described for Example 53 using 8-(1H-imidazol-1-yl)-[1,2,4]triazolo[1,5-a]pyrazine-6-carboxylic acid (Intermediate 16) (30 mg, 0.130 mmol) and (1r,4r)-N¹-(2,2-difluoropropyl)cyclohexane-1,4-diamine hydrochloride (Intermediate 21) (44.71 mg, 0.196 mmol) gave the title compound (4.7 mg, 9% yield) as a white solid. ¹H NMR (400 MHz, DMSO-d₆) 9.40 (s, 1H), 9.33 (s, 1H), 8.98-8.94 (m, 1H), 8.79 (br d, J=1.3 Hz, 1H), 8.63 (br d, J=8.4 Hz, 1H), 7.29 (s, 1H), 3.87 (br d, J=3.3 Hz, 1H), 2.91 (br t, J=13.9 Hz, 3H), 1.97 (br d, J=12.3 Hz, 2H), 1.83 (br d, J=11.4 Hz, 2H), 1.66-1.53 (m, 6H), 1.14 (br d, J=12.0 Hz, 2H). MS ES⁺: 404.4. SFC: Rₜ=2.128 min, 100%.

### Example 58: N-((1R,4r)-4-((R)-3-fluoropyrrolidin-1-yl)cyclohexyl)-2-(1H-imidazol-1-yl)pyrido[2,3-d]pyrimidine-4-carboxamide

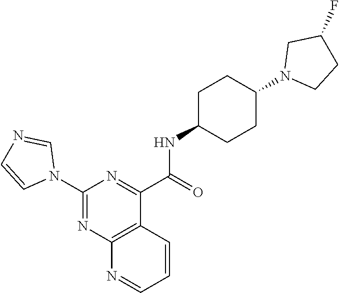 [SMILES: O=C(N[C@H]1CC[C@H](N2CC[C@@H](F)C2)CC1)c1nc(-n2ccnc2)nc2ncccc12]

Following the procedure as described for Example 53 using 2-(1H-imidazol-1-yl)pyrido[2,3-d]pyrimidine-4-carboxylic acid (Intermediate 2) (30 mg, 0.124 mmol) and (1R,4r)-4-((R)-3-fluoropyrrolidin-1-yl)cyclohexan-1-amine (46.3 mg, 0.249 mmol) gave the title compound (6.19 mg, 12% yield) as a white solid. ¹H NMR (400 MHz, DMSO-d₆) 9.37-9.27 (m, 2H), 9.09 (d, J=8.4 Hz, 1H), 9.00 (s, 1H), 8.23 (t, J=1.3 Hz, 1H), 7.78 (dd, J=4.4, 8.3 Hz, 1H), 7.23 (s, 1H), 5.31-5.07 (m, 1H), 3.89 (ttd, J=4.0, 7.7, 11.5 Hz, 1H), 2.94-2.80 (m, 2H), 2.74-2.60 (m, 1H), 2.42-2.33 (m, 1H), 2.11-1.85 (m, 7H), 1.61-1.48 (m, 2H), 1.35-1.23 (m, 2H). MS ES⁺: 410.2. SFC: Rₜ=2.020 min, 100%.

### Example 59: 6-(1H-imidazol-1-yl)-N-((1r,4r)-4-((2,2,2-trifluoroethyl)amino)cyclohexyl)-[1,2,4]triazolo[1,5-a]pyrazine-8-carboxamide

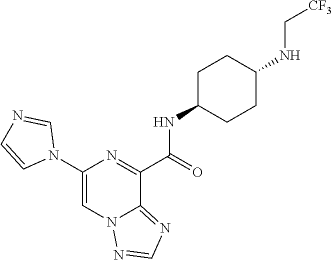 [SMILES: O=C(N[C@H]1CC[C@H](NCC(F)(F)F)CC1)c1nc(-n2ccnc2)cn2ncnc12]

Following the procedure as described for Example 53 using 6-(1H-imidazol-1-yl)-[1,2,4]triazolo[1,5-a]pyrazine-8-carboxylic acid (Intermediate 22) (30 mg, 0.130 mmol) and (1r,4r)-N¹-(2,2,2-trifluoroethyl)cyclohexane-1,4-diamine hydrochloride (30.3 mg, 0.130 mmol) gave the title compound (0.84 mg, 1.5% yield) as a white solid. ¹H NMR (400 MHz, DMSO-d₆) 9.97-9.85 (m, 1H), 8.96-8.90 (m, 1H), 8.88 (s, 1H), 8.70 (s, 1H), 8.14-8.04 (m, 1H), 7.20 (s, 1H), 3.94-3.76 (m, 1H), 3.28-3.22 (m, 3H), 2.29-2.20 (m, 1H), 1.99-1.90 (m, 4H), 1.46 (q, J=11.8 Hz, 2H), 1.22-1.13 (m, 2H). MS ES⁺: 409.2.

### Example 60: 5-(1H-imidazol-1-yl)-N-((1r,4r)-4-((2,2,2-trifluoroethyl)amino)cyclohexyl)-[1,2,4]triazolo[1,5-c]pyrimidine-7-carboxamide

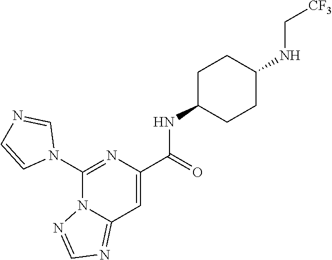 [SMILES: O=C(N[C@H]1CC[C@H](NCC(F)(F)F)CC1)c1cc2ncnn2c(-n2ccnc2)n1]

A mixture of 7-chloro-5-(1H-imidazol-1-yl)-[1,2,4]triazolo[1,5-c]pyrimidine (Intermediate 23) (100 mg, 0.453 mmol), (1r,4r)-N¹-(2,2,2-trifluoroethyl)cyclohexane-1,4-diamine hydrochloride (211 mg, 0.907 mmol), Na₂CO₃ (144 mg, 1.36 mmol), (9,9-dimethyl-9H-xanthene-4,5-diyl)bis(diphenylphosphane) (5.25 mg, 0.009 mmol) and Pd(OAc)₂ (102 mg, 0.453 mmol) in toluene (1 mL) was degassed and purged with CO (3×), and then the mixture was stirred at 80° C. for 17 hours under CO (15 psi) atmosphere. The reaction mixture was concentrated under reduced pressure to give a residue. The residue was purified by prep. HPLC (Column: Phenomenex Luna C18 100×40 mm×3 μm, Mobile Phase A: water (NH₃H₂O+NH₄HCO₃), Mobile Phase B: acetonitrile, Flow rate: 25 mL/min, gradient condition from 6% B to 46%). The pure fractions were collected and the volatiles were removed under vacuum. The residue was partitioned between acetonitrile (2 mL) and water (10 mL) and the mixture was lyophilized to dryness to give the title compound (2.43 mg, 1.3% yield) as a white solid. ¹H NMR (400 MHz, DMSO-d₆) 9.36 (s, 1H), 8.92 (s, 1H), 8.77-8.68 (m, 2H), 8.23 (s, 1H), 7.29 (s, 1H), 3.89-3.83 (m, 1H), 3.27 (br dd, J=3.9, 9.4 Hz, 2H), 2.45 (br d, J=1.5 Hz, 1H), 2.24 (br d, J=5.4 Hz, 1H), 1.97 (br d, J=11.8 Hz, 2H), 1.83 (br d, J=10.6 Hz, 2H), 1.65-1.54 (m, 2H), 1.20-1.12 (m, 2H). MS ES⁺: 409.1.

### Biological Activity

### Human CD38 Hydrolase Assay

The ability of test compounds to inhibit human CD38 hydrolase activity was measured in a fluorescence-based assay using non-physiological NAD⁺ substrate analogue 1,N⁶-etheno NAD⁺ (ε-NAD). Recombinant human CD38 (0.8 nM) was preincubated with test compounds in 384-well black microplates for 30 min at 25° C. in PBS (—Ca²⁺/Mg²⁺) containing 0.005% BSA (pH 7.4). CD38 hydrolase activity was initiated by addition of 4 μM ε-NAD, which yields the fluorescent product 1,N⁶-etheno ADP-ribose. Formation of fluorescent product was followed using ClarioStar Plus (BMG) microplate reader by reading fluorescence (excitation λ=300 nm; emission λ=410 nm) at two time points, one immediately after substrate addition (t=0) and one at 10 min (t=10). Data was analysed by subtracting the values detected at t=0 from t=10 to correct for variation in baseline fluorescence. Fluorescence values were converted to percent inhibition using the average of high signal (CD38 and ε-NAD) and low signal (CD38 and ε-NAD in the presence of a tool CD38 inhibitor) control wells. IC₅₀ values were determined from a 10-point, half log concentration response curve with a four-parameter logistic equation.

### Mouse CD38 Hydrolase Assay

The ability of test compounds to inhibit mouse CD38 hydrolase activity was measured in a fluorescence-based assay using non-physiological NAD⁺ substrate analogue 1,N⁶-etheno NAD⁺ (&-NAD). Recombinant mouse CD38 (0.4 nM) was preincubated with test compounds in 384-well black microplates for 30 min at 25° C. in PBS (—Ca²⁺/Mg²⁺) containing 0.005% BSA (pH 7.4). CD38 hydrolase activity was initiated by addition of 12 μM ε-NAD, which yields the fluorescent product 1,N⁶-etheno ADP-ribose. Formation of fluorescent product was followed using ClarioStar Plus (BMG) microplate reader by reading fluorescence (excitation λ=300 nm; emission λ=410 nm) at two time points, one immediately after substrate addition (t=0) and one at 6 min (t=6).

Data was analysed by subtracting the values detected at t=0 from t=6 to correct for variation in baseline fluorescence. Fluorescence values were converted to percent inhibition using the average of high signal (CD38 and ε-NAD) and low signal (CD38 and ε-NAD in the presence of a tool CD38 inhibitor) control wells. IC₅₀ values were determined from a 10-point, halflog concentration response curve with a four-parameter logistic equation.

The data for human and mouse CD38 activity is summarized in Table 1.

TABLE 1

| | human CD38 | mouse CD38 |
|---|---|---|
| Example | IC₅₀ (μM) | IC₅₀ (μM) |
| 1 | +++ | ++++ |
| 2 | ++++ | ++++ |
| 3 | ++++ | ++++ |
| 4 | ++++ | ++++ |
| 5 | ++ | ++++ |
| 6 | + | ND |
| 7 | ++ | ++++ |
| 8 | ++++ | ++++ |
| 9 | ++++ | ++++ |
| 10 | ++++ | ++++ |
| 11 | ++++ | ++++ |
| 12 | ++++ | ++++ |
| 13 | ++++ | ++++ |
| 14 | ++++ | ++++ |
| 15 | ++++ | ++++ |
| 16 | ++ | ++++ |
| 17 | ++++ | ++++ |
| 18 | ++++ | ++++ |
| 19 | ++++ | ++++ |
| 20 | ++++ | ++++ |
| 21 | ++++ | ++++ |
| 22 | ++ | ++++ |
| 23 | ++++ | ++++ |
| 24 | ++ | ++++ |
| 25 | ++ | ++++ |
| 26 | +++ | ++++ |
| 27 | ++++ | ++++ |
| 28 | + | ++++ |
| 29 | ++++ | ++++ |
| 30 | +++ | ++++ |
| 31 | ++++ | ++++ |
| 32 | +++ | ++++ |
| 33 | ++++ | ++++ |
| 34 | ++ | ND |
| 35 | +++ | ++++ |
| 36 | +++ | ++++ |
| 37 | +++ | ++++ |
| 38 | ++ | ++++ |
| 39 | +++ | ++++ |
| 40 | ++ | ++++ |
| 41 | +++ | ++++ |
| 42 | ++++ | ++++ |
| 43 | ++++ | ND |
| 44 | ++ | ++++ |
| 45 | ++++ | ++++ |
| 46 | +++ | ++++ |
| 47 | +++ | ++++ |
| 48 | +++ | ++++ |
| 49 | ++ | ND |
| 50 | ++ | +++ |
| 51 | ++ | ++++ |
| 52 | +++ | ++++ |
| 53 | ++ | ++++ |
| 54 | ++ | ++++ |
| 55 | ++ | ++++ |
| 56 | +++ | ++++ |
| 57 | ++ | ++++ |
| 58 | +++ | ++++ |
| 59 | ++++ | ++++ |
| 60 | ++ | ND |

IC₅₀ (≤10 μM = ‘+’; ≤1 μM = ‘++’; ≤0.1 μM = ‘+++’; ≤0.04 μM = ‘++++’; ND—not determined)

### Pharmacokinetics

### Tissue Binding Assays

Buffer preparation. A basic solution was prepared by dissolving 14.2 g/L Na₂HPO₄ and 8.77 g/L NaCl in deionized H₂O. An acidic solution was prepared by dissolving 15.6 g/L NaH₂PO₄·2H₂O and 8.77 g/L NaCl in deionized H₂O. Using the acidic solution, the basic solution was then titrated to pH 7.4±0.1 and stored at 4° C. for up to 1 month. The stop solution was 100% CH₃CN containing 200 ng/ml tolbutamide, 200 ng/ml labetalol and 50 ng/ml metformin.

Test method. Dialysis membrane strips were soaked in ultra-pure water at room temperature for ~1 hour. Each membrane strip containing 2 membranes was separated and soaked in 20:80 EtOH/H₂O (v/v) for ~20 min, after which they were ready for use or were stored in the solution at 2-8° C. for up to 1 month. Prior to the experiment, the membrane was rinsed and soaked for 20 min in ultra-pure water. On the day of experiment, brain homogenate was thawed in a water bath at room temperature and incubated at 37° C. for 10 min before use. Test and control compounds were dissolved in DMSO to achieve 10 mM stock solutions. DMSO working solutions were prepared at 400 μM by diluting 10 μL of stock solution. To prepare the time zero (t=0) samples to be used for recovery determination, 50 μL aliquots of loading matrix were transferred in triplicate to the sample collection plate. The samples were immediately matched with opposite blank buffer to obtain a final volume of 100 μL of 1:1 matrix/dialysis buffer (v/v) in each well. 500 μL of stop solution were added to these t=0 samples. They were then stored at 2-8° C. pending further processes along with other post-dialysis samples. To load the dialysis device, an aliquot of 150 μL of the loading matrix was transferred to the donor side of each dialysis well in triplicate, and 150 μL of the dialysis buffer was loaded to the receiver side of the well. The dialysis plate was placed in a humidified incubator at 37° C. with 5% CO₂ on a shaking platform that rotated slowly (about 100 rpm) for 4 hours. At the end of the dialysis, aliquots of 50 μL of samples were taken from both the buffer side and the matrix side of the dialysis device. These samples were transferred into new 96-well plates. Each sample was mixed with an equal volume of opposite blank matrix (buffer or matrix) to reach a final volume of 100 μL of 1:1 matrix/dialysis buffer (v/v) in each well. All samples were further processed by adding 500 μL of stop solution containing internal standards. The mixture was vortexed and centrifuged at 4000 rpm for about 20 min. An aliquot of 100 μL of supernatant of all the samples was then removed for LC-MS/MS analysis. The single blank samples were prepared by transferring 50 μL of blank matrix to a 96-well plate and adding 50 μL of blank PBS buffer to each well. Then the matrix-matched samples were further processed by adding 500 μL of stop solution containing internal standards, following the same sample processing method as the dialysis samples.

Data analysis. The % undiluted unbound, % undiluted bound and % recovery were calculated using the following equations:

$$\% Undiluted Unbound = 100 * 1 / D / ( ( 1 / ( F / T ) - 1 ) + 1 / D )$$

$$\% Undiluted Bound = 100 - \% Undiluted Unbound$$

$$\% Recovery = 100 * ( F + T ) / To$$

where F is the analyte concentration or peak area ratio of analyte/internal standard on the buffer (receiver) side of the membrane; T is the analyte concentration or peak area ratio of analyte/internal standard on the matrix (donor) side of the membrane; To is the analyte concentration or the peak area ratio of analyte/internal standard in the loading matrix sample at time zero; and D is the dilution factor determined as 4 in this assay.

Unbound fractions in brain homogenate and plasma for selected compounds are summarized in Table 2.

TABLE 2

| | Mouse brain | Mouse plasma |
|---|---|---|
| Example | (% unbound) | (% unbound) |
| 4 | 41 | 40 |
| 12 | 6.8 | 6.2 |
| 13 | 5.6 | 22 |
| 14 | 5.6 | 24 |
| 19 | 25 | 19 |
| 20 | 34 | 14 |
| 21 | 34 | 30 |
| 29 | 6.4 | 4.4 |
| 41 | 15 | 20 |
| 45 | 15 | 20 |
| 48 | 15 | 24 |

### PK Brain Permeability

The distribution of compounds into the brain in vivo was determined in C57BL/6 mice following single oral (po) gavage administration.

Test compounds were formulated at 1 mg/mL in 0.5% HPMC E4M, 0.2% Tween 80 in water to achieve solutions or homogenous suspensions suitable for po administration. Formulations were administered to 3 male C₅₇BL/6 mice at a volume of 10 mL/kg resulting in a dose level of 10 mg/kg. Blood samples were collected at 1 and 2 hours post dose into tubes containing K₂EDTA as anticoagulant, processed to plasma and stored at −60° C. or lower until LC-MS/MS analysis. Brains were harvested 2 hours post dose, rinsed with saline, dried, weighed and homogenised under ice cold conditions. Brain homogenates were stored at −60° C. or lower until LC-MS/MS analysis.

Dose formulation concentrations were verified using a LC-UV or LC-MS/MS method. Test compound concentrations in plasma and brain homogenate were quantitatively determined using LC-MS/MS methods developed in individual matrices against calibration curves with QC samples included and acceptance criteria as per CRO SOPs. Concentrations in brain homogenate were corrected for the dilution factor used to prepare the homogenate to give concentrations in whole brain tissue.

Plasma and brain concentration versus time data were reported and plotted in excel. The brain to plasma ratio at 2 hours post dose was calculated for each animal using the following equation:

Brain: plasma=brain concentration (ng/g) at 2 h/plasma concentration (ng/ml) at 2 h As only unbound test compound is available to exert an effect on the target, unbound plasma (Cp_{,u}) and unbound brain (Cb_{,u}) concentrations were calculated by correcting the total concentrations for the unbound fraction in plasma (fu_{,p}) or brain (fu_{,b}) determined from in vitro plasma protein or brain tissue binding assays using the following equations:

$$Cp , u = plasma concentration * fu , p$$

$$Cb , u = brain concentration * fu , b$$

The unbound partitioning coefficient (Kp_{u,u}) was then calculated based on the ratio of Cb_{,u} to Cp_{,u} using the following equation:

$${Kp}_{, uu} = C {b}_{, u} / {Cp}_{, u}$$

Selected data from pharmacokinetic studies are summarized in Table 3. In these studies, mice were orally administrated a 10 mg/kg dose and sacrificed 2 hours post dose and tissue samples (brain homogenate and plasma) were analyzed subsequently as described above.

TABLE 3

| | total | free | total | free | |
|---|---|---|---|---|---|
| | plasma | plasma | brain | brain | |
| | conc. | conc. | conc. | conc. | |
| Example | (nM) | (nM) | (nM) | (nM) | Kp_{u, u} |
| 4 | 6311 | 2535 | 572 | 236 | 0.09 |
| 12 | 12608 | 788 | 8368 | 575 | 0.79 |
| 13 | 3663 | 809 | 3379 | 189 | 0.23 |
| 14 | 3932 | 959 | 3323 | 181 | 0.19 |
| 19 | 5014 | 954 | 864 | 218 | 0.22 |
| 20 | 17882 | 2626 | 1420 | 481 | 0.18 |
| 21 | 8215 | 2470 | 1502 | 507 | 0.20 |
| 29 | 19111 | 840 | 4918 | 319 | 0.39 |
| 41 | 10638 | 2164 | 1584 | 241 | 0.11 |
| 45 | 9341 | 3494 | 2164 | 555 | 0.16 |
| 48 | 7996 | 1985 | 2569 | 403 | 0.20 |

The data in Table 3 illustrates that compounds disclosed herein such as Examples 4, 12, 13, 14, 19, 20, 21, 29, 41, 45 and 48 display good brain permeability.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 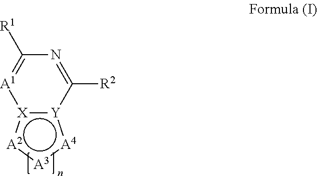
or a pharmaceutically acceptable salt, solvate or prodrug thereof, wherein:
one of R¹ and R² is —C(O)NHR³ and the other one of R¹ and R² is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
R³ is a saturated 3- to 9-membered carbocyclic or heterocyclic group optionally substituted with one or more substituents independently selected from —NR⁴R⁵ and C₁-C₃ alkyl, wherein the C₁-C₃ alkyl is optionally substituted with one or more substituents independently selected from halo, hydroxyl and C₁-C₃ alkoxy;
R⁴ is hydrogen or C₁-C₃ alkyl;
R⁵ is C₁-C₄ alkyl optionally substituted with one or more substituents independently selected from halo, hydroxyl and C₁-C₃ alkoxy;
or R⁴ and R⁵ together with the nitrogen atom to which they are attached form a saturated 3- to 6-membered heterocyclic group, wherein the saturated 3- to 6-membered heterocyclic group is optionally substituted with one or more substituents independently selected from halo, hydroxyl, C₁-C₃ alkyl, C₁-C₃ haloalkyl, C₁-C₃ alkoxy and oxo (═0);
each of A¹, A², A³ and A⁴ is independently selected from N and CH;
each X and Y is independently selected from N and C; and
n is 1 or 2;
provided that:
when n is 1, one of X and Y is N and the other one of X and Y is C; and
when n is 2, X and Y are C and at least one of A², A³ and A⁴ is N.

2. The compound, salt, solvate or prodrug as claimed in claim 1, wherein the compound is of Formula (Ia): 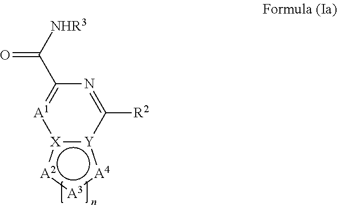
or a pharmaceutically acceptable salt, solvate or prodrug thereof, wherein:
R² is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
R³ is a saturated 3- to 9-membered carbocyclic or heterocyclic group optionally substituted with one or more substituents independently selected from —NR⁴R⁵ and C₁-C₃ alkyl, wherein the C₁-C₃ alkyl is optionally substituted with one or more substituents independently selected from halo, hydroxyl and C₁-C₃ alkoxy;
R⁴ is hydrogen or C₁-C₃ alkyl;
R⁵ is C₁-C₄ alkyl optionally substituted with one or more substituents independently selected from halo, hydroxyl and C₁-C₃ alkoxy;
or R⁴ and R⁵ together with the nitrogen atom to which they are attached form a saturated 3- to 6-membered heterocyclic group, wherein the saturated 3- to 6-membered heterocyclic group is optionally substituted with one or more substituents independently selected from halo, hydroxyl, C₁-C₃ alkyl, C₁-C₃ haloalkyl, C₁-C₃ alkoxy and oxo (═O);
each of A¹, A², A³ and A⁴ is independently selected from N and CH;
each X and Y is independently selected from N and C; and
n is 1 or 2;
provided that:
when n is 1, one of X and Y is N and the other one of X and Y is C; and
when n is 2, X and Y are C and at least one of A², A³ and A⁴ is N.

3. The compound, salt, solvate or prodrug as claimed in claim 1, wherein the compound is of Formula (Ib): 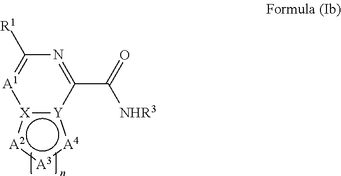
or a pharmaceutically acceptable salt, solvate or prodrug thereof, wherein:
R¹ is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
R³ is a saturated 3- to 9-membered carbocyclic or heterocyclic group optionally substituted with one or more substituents independently selected from —NR⁴R⁵ and C₁-C₃ alkyl, wherein the C₁-C₃ alkyl is optionally substituted with one or more substituents independently selected from halo, hydroxyl and C₁-C₃ alkoxy;
R⁴ is hydrogen or C₁-C₃ alkyl;
R⁵ is C₁-C₄ alkyl optionally substituted with one or more substituents independently selected from halo, hydroxyl and C₁-C₃ alkoxy;
or R⁴ and R⁵ together with the nitrogen atom to which they are attached form a saturated 3- to 6-membered heterocyclic group, wherein the saturated 3- to 6-membered heterocyclic group is optionally substituted with one or more substituents independently selected from halo, hydroxyl, C₁-C₃ alkyl, C₁-C₃ haloalkyl, C₁-C₃ alkoxy and oxo (═O);
each of A¹, A², A³ and A⁴ is independently selected from N and CH;
each X and Y is independently selected from N and C; and
n is 1 or 2;
provided that:
when n is 1, one of X and Y is N and the other one of X and Y is C; and
when n is 2, X and Y are C and at least one of A², A³ and A⁴ is N.

4. The compound, salt, solvate or prodrug as claimed in claim 1, wherein n is 1.

5. The compound, salt, solvate or prodrug as claimed in claim 1, wherein n is 2.

6. The compound, salt, solvate or prodrug as claimed in claim 1, wherein one or two of A¹, A², A³ and A⁴ are N and the remaining of A¹, A², A³ and A⁴ are CH.

7. The compound, salt, solvate or prodrug as claimed in claim 1, wherein A³ is CH.

8. The compound, salt, solvate or prodrug as claimed in claim 1, wherein the 5-membered heteroaryl group of R¹ or R² is selected from pyrazolyl, imidazolyl, thiazolyl, isothiazolyl, triazolyl, and thiadiazolyl, each of which is optionally substituted with one or two substituents independently selected from C₁-C₃ alkyl, wherein the C₁-C₃ alkyl is optionally substituted with one, two, three, four or five halo substituents or with one substituent selected from hydroxyl and C₁-C₃ alkoxy.

9. The compound, salt, solvate or prodrug as claimed in claim 1, wherein the saturated 3- to 9-membered carbocyclic or heterocyclic group of R³ is selected from cyclopropyl, cyclobutyl, cyclopentyl, cyclohexyl, cycloheptyl, cyclooctyl, azetidinyl, oxetanyl, thietanyl, pyrrolidinyl, tetrahydrofuranyl, tetrahydrothiophenyl, pyrazolidinyl, imidazolidinyl, oxazolidinyl, isoxazolidinyl, dioxolanyl, oxathiolanyl, piperidinyl, tetrahydropyranyl, thianyl, piperazinyl, dioxanyl, morpholinyl, thiomorpholinyl, spiro[2.3]hexanyl, spiro[2.4]heptanyl, spiro[3.3]heptanyl, spiro[2.5]octanyl, spiro[3.4]octanyl, spiro[2.6]nonanyl, spiro[3.5]nonanyl, spiro[4.4]nonanyl, azaspiro[2.3]hexanyl, azaspiro[2.4]heptanyl, azaspiro[3.3]heptanyl, azaspiro[2.5]octanyl, azaspiro[3.4]octanyl, azaspiro[2.6]nonanyl, azaspiro[3.5]nonanyl, and azaspiro[4.4]nonanyl, each of which is optionally substituted with one or two substituents independently selected from:
(i) C₁-C₃ alkyl optionally substituted with one, two, three, four or five halo substituents or with one substituent selected from hydroxyl and C₁-C₃ alkoxy;
(ii) —NR⁴R⁵, wherein:
R⁴ is hydrogen or C₁-C₃ alkyl; and
R⁵ is C₁-C₄ alkyl optionally substituted with one, two, three, four or five halo substituents or with one substituent selected from hydroxyl and C₁-C₃ alkoxy; and
(iii) azetidinyl, pyrrolidinyl, pyrazolidinyl, imidazolidinyl, oxazolidinyl, isoxazolidinyl, piperidinyl, piperazinyl, morpholinyl, and thiomorpholinyl, each of which is optionally substituted with one, two, three, four or five substituents independently selected from halo, hydroxyl, C₁-C₃ alkyl, C₁-C₃ haloalkyl, C₁-C₃ alkoxy and oxo (═O).

10. The compound, salt, solvate or prodrug as claimed in claim 1, wherein the compound is selected from:
7-(1H-imidazol-1-yl)-N-(tetrahydro-2H-pyran-4-yl) imidazo[1,2-c]pyrimidine-5-carboxamide;
7-(1H-imidazol-1-yl)-N-((1r,4r)-4-((2,2,2-trifluoroethyl)amino)cyclohexyl) imidazo[1,2-c]pyrimidine-5-carboxamide;
2-(1H-imidazol-1-yl)-N-(tetrahydro-2H-pyran-4-yl)pyrido[2,3-d]pyrimidine-4-carboxamide;
2-(1H-imidazol-1-yl)-N-((1r,4r)-4-((2,2,2-trifluoroethyl)amino)cyclohexyl)pyrido[2,3-d]pyrimidine-4-carboxamide;
3-(thiazol-5-yl)-N-((1r,4r)-4-((2,2,2-trifluoroethyl)amino)cyclohexyl)pyrrolo[1,2-a]pyrazine-1-carboxamide;
8-(1H-imidazol-1-yl)-N-((1r,4r)-4-((2,2,2-trifluoroethyl)amino)cyclohexyl)-1,7-naphthyridine-6-carboxamide;
8-(thiazol-5-yl)-N-((1r,4r)-4-((2,2,2-trifluoroethyl)amino)cyclohexyl)-1,7-naphthyridine-6-carboxamide;
2-(1H-imidazol-1-yl)-N-(2-(2,2,2-trifluoroethyl)-2-azaspiro[3.5]nonan-7-yl)pyrrolo[2,1-f][1,2,4]triazine-4-carboxamide;
2-(1H-imidazol-1-yl)-N-(6-((2,2,2-trifluoroethyl)amino)spiro[3.3]heptan-2-yl)pyrrolo[2,1-f][1,2,4]triazine-4-carboxamide;
2-(1H-imidazol-1-yl)-N-(6-((2,2,2-trifluoroethyl)amino)spiro[3.3]heptan-2-yl)pyrrolo[2,1-f][1,2,4]triazine-4-carboxamide;
2-(1H-imidazol-1-yl)-N-((1r,3r)-3-((2,2,2-trifluoroethyl)amino)cyclobutyl)pyrrolo[2,1-f][1,2,4]triazine-4-carboxamide;
N-((1r,4r)-4-(3,3-difluoropyrrolidin-1-yl)cyclohexyl)-2-(1H-imidazol-1-yl)pyrrolo[2,1-f][1,2,4]triazine-4-carboxamide;
N-((1S,4r)-4-((S)-3-fluoropyrrolidin-1-yl)cyclohexyl)-2-(1H-imidazol-1-yl)pyrrolo[2,1-f][1,2,4]triazine-4-carboxamide;
N-((1R,4r)-4-((R)-3-fluoropyrrolidin-1-yl)cyclohexyl)-2-(1H-imidazol-1-yl)pyrrolo[2,1-f][1,2,4]triazine-4-carboxamide;
1-(1H-imidazol-1-yl)-N-((1r,4r)-4-((2,2,2-trifluoroethyl)amino)cyclohexyl)pyrrolo[1,2-a]pyrazine-3-carboxamide;
4-(1H-imidazol-1-yl)-N-((1r,4r)-4-((2,2,2-trifluoroethyl)amino)cyclohexyl)pyrrolo[2,1-f][1,2,4]triazine-2-carboxamide;
2-(1-methyl-1H-imidazol-5-yl)-N-((1r,4r)-4-((2,2,2-trifluoroethyl)amino)cyclohexyl)pyrrolo[2,1-f][1,2,4]triazine-4-carboxamide;
2-(1-(2-hydroxyethyl)-1H-imidazol-5-yl)-N-((1r,4r)-4-((2,2,2-trifluoroethyl)amino)cyclohexyl)pyrrolo[2,1-f][1,2,4]triazine-4-carboxamide;
8-(1H-imidazol-1-yl)-N-((1r,4r)-4-((2,2,2-trifluoroethyl)amino)cyclohexyl) imidazo[1,2-a]pyrazine-6-carboxamide;
8-(1H-imidazol-1-yl)-N-((1r,4r)-4-((2,2,2-trifluoroethyl)amino)cyclohexyl)-[1,2,4]triazolo[1,5-a]pyrazine-6-carboxamide;
4-(1H-imidazol-1-yl)-N-((1r,4r)-4-((2,2,2-trifluoroethyl)amino)cyclohexyl)pyrazolo[1,5-a]pyrazine-6-carboxamide;
N-((1S,4r)-4-((S)-3-fluoropyrrolidin-1-yl)cyclohexyl)-1-(1H-imidazol-1-yl)pyrrolo[1,2-a]pyrazine-3-carboxamide;
N-((1S,4r)-4-((S)-3-hydroxypyrrolidin-1-yl)cyclohexyl)-2-(1H-imidazol-1-yl)pyrrolo[2,1-f][1,2,4]triazine-4-carboxamide;
N-((1R,4r)-4-((R)-3-fluoropyrrolidin-1-yl)cyclohexyl)-1-(1H-imidazol-1-yl)pyrrolo[1,2-a]pyrazine-3-carboxamide;
N-((1S,4r)-4-((S)-3-fluoropyrrolidin-1-yl)cyclohexyl)-8-(1H-imidazol-1-yl)-[1,2,4]triazolo[1,5-a]pyrazine-6-carboxamide;
N-((1R,4r)-4-((R)-3-fluoropyrrolidin-1-yl)cyclohexyl)-8-(1H-imidazol-1-yl)-[1,2,4]triazolo[1,5-a]pyrazine-6-carboxamide;
N-((1R,4r)-4-((R)-3-hydroxypyrrolidin-1-yl)cyclohexyl)-2-(1H-imidazol-1-yl)pyrrolo[2,1-f][1,2,4]triazine-4-carboxamide;
N-((1R,4r)-4-((R)-3-fluoropyrrolidin-1-yl)cyclohexyl)-4-(1H-imidazol-1-yl)pyrrolo[2,1-f][1,2,4]triazine-2-carboxamide;
2-(1H-imidazol-1-yl)-N-((1r,4r)-4-((2,2,2-trifluoroethyl)amino)cyclohexyl)pyrrolo[2,1-f][1,2,4]triazine-4-carboxamide;
2-(thiazol-5-yl)-N-((1r,4r)-4-((2,2,2-trifluoroethyl)amino)cyclohexyl)pyrrolo[2,1-f][1,2,4]triazine-4-carboxamide;
N-((1r,4r)-4-((2,2-difluoroethyl)amino)cyclohexyl)-2-(1H-imidazol-1-yl)pyrrolo[2,1-f][1,2,4]triazine-4-carboxamide;
N-((1S,4r)-4-((S)-3-fluoropyrrolidin-1-yl)cyclohexyl)-2-(1H-imidazol-1-yl)pyrido[2,3-d]pyrimidine-4-carboxamide;
N-((1r,4r)-4-((2,2-difluoroethyl)amino)cyclohexyl)-2-(1H-imidazol-1-yl)pyrido[2,3-d]pyrimidine-4-carboxamide;
N-((1r,4r)-4-(3,3-difluoropyrrolidin-1-yl)cyclohexyl)-4-(1H-imidazol-1-yl)pyrazolo[1,5-a]pyrazine-6-carboxamide;
N-((1R,4r)-4-((R)-3-fluoropyrrolidin-1-yl)cyclohexyl)-4-(1H-imidazol-1-yl)pyrazolo[1,5-a]pyrazine-6-carboxamide;
N-((1S,4r)-4-((S)-3-fluoropyrrolidin-1-yl)cyclohexyl)-4-(1H-imidazol-1-yl)pyrazolo[1,5-a]pyrazine-6-carboxamide;
N-((1r,4r)-4-((2,2-difluoroethyl)amino)cyclohexyl)-4-(1H-imidazol-1-yl)pyrazolo[1,5-a]pyrazine-6-carboxamide;
N-((1r,4r)-4-((2,2-difluoroethyl)amino)cyclohexyl)-8-(1H-imidazol-1-yl) imidazo[1,2-a]pyrazine-6-carboxamide;
N-((1R,4r)-4-((R)-3-fluoropyrrolidin-1-yl)cyclohexyl)-8-(1H-imidazol-1-yl) imidazo[1,2-a]pyrazine-6-carboxamide;
N-((1S,4r)-4-((S)-3-fluoropyrrolidin-1-yl)cyclohexyl)-8-(1H-imidazol-1-yl) imidazo[1,2-a]pyrazine-6-carboxamide;
N-((1r,4r)-4-(3,3-difluoropyrrolidin-1-yl)cyclohexyl)-8-(1H-imidazol-1-yl) imidazo[1,2-a]pyrazine-6-carboxamide;
2-(5-methyl-1H-imidazol-1-yl)-N-((1r,4r)-4-((2,2,2-trifluoroethyl)amino)cyclohexyl)pyrrolo[2,1-f][1,2,4]triazine-4-carboxamide;
2-(1H-imidazol-1-yl)-N-((1r,4r)-4-(pyrrolidin-1-yl)cyclohexyl)pyrrolo[2,1-f][1,2,4]triazine-4-carboxamide;
2-(1H-imidazol-1-yl)-N-((1s,4s)-4-(pyrrolidin-1-yl)cyclohexyl)pyrrolo[2,1-f][1,2,4]triazine-4-carboxamide;
N-((1r,4r)-4-(3,3-difluoroazetidin-1-yl)cyclohexyl)-8-(1H-imidazol-1-yl)-[1,2,4]triazolo[1,5-a]pyrazine-6-carboxamide;
N-((1r,4r)-4-(3-fluoro-3-methylazetidin-1-yl)cyclohexyl)-8-(1H-imidazol-1-yl)-[1,2,4]triazolo[1,5-a]pyrazine-6-carboxamide;
8-(1H-imidazol-1-yl)-N-((1r,4r)-4-(methyl (2,2,2-trifluoroethyl)amino)cyclohexyl)-[1,2,4]triazolo[1,5-a]pyrazine-6-carboxamide;
N-((1r,4r)-4-(3,3-difluoropyrrolidin-1-yl)cyclohexyl)-8-(1H-imidazol-1-yl)-[1,2,4]triazolo[1,5-a]pyrazine-6-carboxamide;
8-(1H-imidazol-1-yl)-N-((1r,4r)-4-morpholinocyclohexyl)-[1,2,4]triazolo[1,5-a]pyrazine-6-carboxamide;
8-(1H-imidazol-1-yl)-N-(1-(2,2,2-trifluoroethyl) piperidin-4-yl)-[1,2,4]triazolo[1,5-a]pyrazine-6-carboxamide;
N-((1r,4r)-4-(1,1-dioxidothiomorpholino)cyclohexyl)-8-(1H-imidazol-1-yl)-[1,2,4]triazolo[1,5-a]pyrazine-6-carboxamide;
N-((1r,4r)-4-((2,2-difluoroethyl)amino)cyclohexyl)-8-(1H-imidazol-1-yl)-[1,2,4]triazolo[1,5-a]pyrazine-6-carboxamide;
8-(1H-imidazol-1-yl)-N-((1r,4r)-4-((1,1,1-trifluoro-2-methylpropan-2-yl)amino)cyclohexyl)-[1,2,4]triazolo[1,5-a]pyrazine-6-carboxamide;
8-(1H-imidazol-1-yl)-N-((1S,4s)-4-((R)-3-methoxypyrrolidin-1-yl)cyclohexyl)-[1,2,4]triazolo[1,5-a]pyrazine-6-carboxamide;
8-(1H-imidazol-1-yl)-N-((1s,4s)-4-thiomorpholinocyclohexyl)-[1,2,4]triazolo[1,5-a]pyrazine-6-carboxamide;
8-(1H-imidazol-1-yl)-N-((1r,4r)-4-(3-(trifluoromethyl) azetidin-1-yl)cyclohexyl)-[1,2,4]triazolo[1,5-a]pyrazine-6-carboxamide;
N-((1r,4r)-4-((2,2-difluoropropyl)amino)cyclohexyl)-8-(1H-imidazol-1-yl)-[1,2,4]triazolo[1,5-a]pyrazine-6-carboxamide;
N-((1R,4r)-4-((R)-3-fluoropyrrolidin-1-yl)cyclohexyl)-2-(1H-imidazol-1-yl)pyrido[2,3-d]pyrimidine-4-carboxamide;
6-(1H-imidazol-1-yl)-N-((1r,4r)-4-((2,2,2-trifluoroethyl)amino)cyclohexyl)-[1,2,4]triazolo[1,5-a]pyrazine-8-carboxamide;
5-(1H-imidazol-1-yl)-N-((1r,4r)-4-((2,2,2-trifluoroethyl)amino)cyclohexyl)-[1,2,4]triazolo[1,5-c]pyrimidine-7-carboxamide;
or an enantiomer of any of the foregoing;
or a pharmaceutically acceptable salt, solvate or prodrug of any of the foregoing.

11. A process for the preparation of a compound, salt, solvate or prodrug as claimed in claim 1, wherein the process comprises:
(a) the step of reacting a compound of formula (II) or a salt thereof with an amine of formula (III) or a salt thereof or protected derivative thereof: 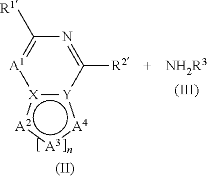
wherein:
one of R^{1′} and R^{2′} is —C(O) Z and the other one of R^{1′} and R^{2′} is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
Z is —OH, —OR⁶, —O—CO—R⁶, —F or —Cl;
R⁶ is C₁-C₃ alkyl; and
R³, n, A¹, A², A³, A⁴, X and Y are as defined in any one of claim 2; or
(b) the step of reacting a compound of formula (IV) or a salt thereof with a compound of formula (V) or a salt thereof: 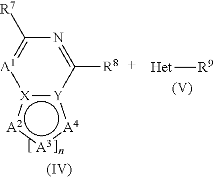
wherein:
one of R⁷ and R⁸ is —CONHR³ and the other one of R⁷ and R⁸ is a leaving group;
Het is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy;
R⁹ is a leaving group, —Sn(C₁-C₄ alkyl)₃ or —B(R¹⁰)₂;
each R¹⁰ is independently selected from hydroxyl, C₁-C₅ alkoxy and C₁-C₅ alkyl, or two R¹⁰ together with the boron atom to which they are attached form an optionally substituted 5- to 6-membered heterocyclic group; and
R³, n, A¹, A², A³, A⁴, X and Y are as defined in claim 1; or
(c) the step of reacting a compound of formula (VI) or a salt thereof with an amine of formula (III) or a salt thereof or protected derivative thereof: 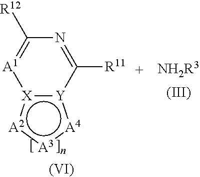
wherein:
one of R¹¹ and R¹² is a 5-membered heteroaryl group containing one, two or three heteroatoms independently selected from N and S, wherein the heteroaryl group is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy, and the other one of R¹¹ and R¹² is a leaving group; and
R³, n, A¹, A², A³, A⁴, X and Y are as defined in claim 1; or
(d) the step of reacting a compound of formula (IV) or a salt thereof with a compound of formula (VII) or a salt thereof: 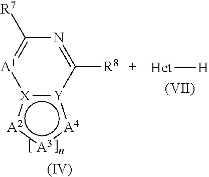
wherein:
one of R⁷ and R⁸ is —CONHR³ and the other one of R⁷ and R⁸ is a leaving group;
Het-H is a heteroaryl compound selected from 1H-pyrrole, 1H-imidazole, 1H-pyrazole, 1H-1,2,3-triazole, 2H-1,2,3-triazole, 1H-1,2,4-triazole and 4H-1,2,4-triazole, wherein the heteroaryl compound is optionally substituted with one or more substituents independently selected from C₁-C₃ alkyl, wherein the C₁-C₃ alkyl is optionally substituted with one or more substituents independently selected from halo, hydroxyl and C₁-C₃ alkoxy; and
R³, n, A¹, A², A³, A⁴, X and Y are as defined in claim 1;
and optionally thereafter carrying out one or more of the following procedures:
converting a compound of formula (I), (Ia), (Ib), (I′), (Ia′), (Ib′), (I″), (Ia″) or (Ib″) into another compound of formula (I), (Ia), (Ib), (I′), (Ia′), (Ib′), (I″), (Ia″) or (Ib″);
removing any protecting groups;
forming a pharmaceutically acceptable salt.

12. A pharmaceutical composition comprising a compound, salt, solvate or prodrug as claimed in claim 1, in association with a pharmaceutically acceptable adjuvant, diluent or carrier, and optionally one or more other therapeutic agents.

13. The compound, salt, solvate or prodrug as claimed in claim 1, for use in therapy.

14. A method of treating or preventing a disease, disorder or condition associated with CD38 activity, the method comprising administering to a patient in need thereof a therapeutically or prophylactically effective amount of a compound, salt, solvate or prodrug as claimed in claim 1.

15. A method of treating or preventing a CNS disease, a disease requiring treatment via the CNS, a neurodegenerative condition, a neurological disease, an age-related disorder, or an inflammatory disorder, the method comprising administering to a patient in need thereof a therapeutically or prophylactically effective amount of a compound, salt, solvate or prodrug as claimed in claim 1.

16. A method of treating or preventing Parkinson's disease; Alzheimer's disease; frontotemporal dementia; progressive supranuclear palsy; a tauopathy; another non-Alzheimer's dementia; stroke; ischemic insult; traumatic brain injury; multiple sclerosis; an autoimmune disease with associated neuronal damage such as Muckle-Wells syndrome; motor neuron disease such as amyotrophic lateral sclerosis; axonal neuropathy or axonal degeneration such as diabetic neuropathy; Wallerian degeneration; ataxia telangiectasia; Friedreich's ataxia; another ataxia such as spinocerebellar ataxia 7; aging; senescence; neuroinflammation; depression; schizophrenia; anxiety; stress; post-traumatic stress disorder; glaucoma; age-related macular degeneration; hearing loss; an autoimmune disease such as rheumatoid arthritis or Lupus; obesity; or metabolic syndrome, the method comprising administering to a patient in need thereof a therapeutically or prophylactically effective amount of a compound, salt, solvate or prodrug as claimed in claim 1.
